(19) United States
(12) Patent Application Publication
(10) Pub. No.: US 2018/0148793 A1
(43) Pub. Date: May 31, 2018

(54) BIOMARKERS AND USES THEREOF FOR SELECTING PANCREAS CANCER INTERVENTION

(71) Applicant: Fred Hutchinson Cancer Research Center, Seattle, WA (US)

(72) Inventor: Sunil R. HINGORANI, Seattle, WA (US)

(21) Appl. No.: 15/575,222

(22) PCT Filed: May 19, 2016
(86) PCT No.: PCT/US2016/033378

§ 371 Date: Nov. 17, 2017

Related U.S. Application Data
(60) Provisional application No. 62/163,842, filed on May 19, 2015.

(51) Int. Cl. C12Q 1/6886; A61P 35/00; A61P 1/18

(52) U.S. Cl. CPC C12Q 1/6886; A61P 35/00; C12Q 2600/118; C12Q 2600/156; C12Q 2600/158; A61P 1/18

(57) ABSTRACT

The instant disclosure provides methods for using biomarkers for identifying therapeutic regimens to treat pancreas cancer, or to reduce the risk of pancreas cancer, pancreas cancer metastasis, pancreas cancer proliferation, or pancreas cancer recurrence. For example, such therapeutic regimens can be based on the level of RUNX3 expression in the context of a particular DPC4 genotype, or based on the level of RUNX3 expression in combination with the level of Col6a1 expression, Spp1 expression or both. Such therapeutic regimens can include a main therapy, a neoadjuvant therapy, an adjuvant therapy, or any combination thereof. Also provided are methods for diagnosing or detecting metastatic potential of pancreas cancer cells based on the foregoing.

### STATEMENT OF GOVERNMENT INTEREST

This invention was made with government support under Grant Nos. CA129537, CA161112 and CA114028 awarded by the National Institutes of Health. The government has certain rights in the invention.

### STATEMENT REGARDING SEQUENCE LISTING

The Sequence Listing associated with this application is provided in text format in lieu of a paper copy, and is hereby incorporated by reference into the specification. The name of the text file containing the Sequence Listing is 360056_436WO_SEQUENCE_LISTING.txt. The text file is 3.6 KB, was created on May 18, 2016, and is being submitted electronically via EFS-Web.

### BACKGROUND

Patients with carcinomas die primarily of metastatic disease. This is especially true of pancreatic ductal adenocarcinoma (PDA), which is notorious for its early and extreme penchant for metastatic spread. A minority of patients instead presents with and succumbs to locally advanced disease, although the reasons for these distinct presentations remain unknown. PDA has either overtly metastasized or advanced locally beyond the boundaries of surgical resection in most patients at the time of diagnosis; subsequent median survival is approximately 4.5 and 10.6 months, respectively (Hidalgo, New England J. Med. 362:1605, 2010). For the fortunate few for whom surgical resection is possible, median survival increases to 2 years but is not durable; survival at 5 years is only 20% and declines to less than 2% at 10 years (Allison et al., J. Surg. Oncol. 67:151, 1998). The majority of these post-operative patients also eventually die of metastatic disease suggesting that clinical Stage I tumors are, in fact, already micrometastatic Stage IV.

In a uniformly lethal disease, prognosis per se may be less informative for rational treatment design than predicting disease behaviors and likely proximal cause of death. Indeed, neoadjuvant strategies for early stage PDA were introduced because many patients ultimately die from distant relapse after surgery; addressing this reality with a course of chemotherapy prior to resection can prolong survival, but runs the attendant risk of local tumor growth beyond surgical boundaries and therefore a lost chance for cure. Thus, knowing when to operate or irradiate and when to treat systemically remains unclear.

PDA begins most commonly in precursor lesions termed pancreatic intraepithelial neoplasms (PanIN) that arise in terminal ductules (Hruban et al., Am. J. Surg. Pathol. 25:579, 2001). Activating KRAS mutations occur early in preinvasive disease and are almost uniformly present (>90%) in invasive PDA. Mutations in CDKN2A/INK4A are similarly abundant in invasive disease (>95%) and point mutations in TP53 are also common (>75%). Loss of DPC4/SMAD4 expression, the last of the principal genetic events associated with PDA, occurs late in PanIN-to-PDA progression and is seen in approximately 50% of invasive cancers (Iacobuzio-Donahue et al., Am. J. Surg. Pathol. 24:1544, 2000).

Various studies indicate that distinct combinations of tumor suppressor gene mutations can alter the pace, phenotype and prognosis of the resultant invasive disease. For example, concomitant mutations in p16/p19 (Aguirre et al., Genes Dev. 17:3112, 2003) or Trp53 (Hingorani et al., Cancer Cell 7:469, 2005) hasten progression initiated by oncogenic Kras (Hingorani et al., Cancer Cell 4:437, 2003), albeit with distinct characteristics. In the case of biallelic p16/p19 loss, the primary tumor progresses rapidly, leading to early death and minimal metastatic disease. Kras^{LSL-G12D/+}; Trp53^{LSL-R172H/+}; Cre (KPC) animals instead succumb from a combination of both primary and metastatic tumor burdens that closely mimics the common presentation in humans. In contrast, heterozygous deletion of Dpc4/Smad4 in the context of oncogenic Kras^{G12D} expression generates macroscopic cystic precursors known as mucinous cystic neoplasms (MCN) (Izeradjene et al., Cancer Cell 11:229, 2007). Subsequent progression to invasive PDA occurs through additional spontaneous events, including loss of heterozygosity (LOH) of Dpc4, but the disease manifests an attenuated metastatic potential. Invasive PDA that arise from MCN in patients also invariably lose DPC4 expression (Iacobuzio-Donahue et al., 2000) and have a better prognosis than cancers following the more common PanIN-to-PDA route (Crippa et al., Ann Surg. 247:571, 2008).

Hence, there remains a need in the art for predicting pancreas cancer behavior in order to inform rational treatment decisions to promote or maximize survival. The present disclosure meets such needs, and further provides other related advantages.

### BRIEF DESCRIPTION OF THE DRAWINGS

FIG. 1. Molecular characterization of tumor progression in KPDC mice

(A) Kaplan-Meier survival of KPDC animals (167 days) was significantly less than control animals (769 days, p<0.001), Kras^{LSL-G12D/+}; Dpc4^{flox/+}; p48^{Cre/+} (KDC) mice (479 days, p<0.001) and KPC mice (209 days, p<0.05) (log rank test for each pairwise combination).

(B and C) Gross pathology of KPDC pancreata at necropsy. Dashed lines, tumor.

(D) Representative KPDC tumor.

(E) Representative KPC tumor.

(F) Pancreas histology in young KPDC animal (age=109 days). Arrow, PanIN-1A.

(G) Pancreas histology in older KPDC animal (age=165 days). Arrow, PanIN-3.

(H) Moderately well-differentiated KPDC PDA.

(I) Poorly differentiated KPDC PDA.

(J) CK-19 immunoreactivity highlights KPDC ductal epithelium. Arrows, PanIN-1A; arrowhead, normal duct.

(K) Alcian blue histochemistry (arrow) reveals mucin content in preinvasive KPDC lesion.

(L) Metastatic potential (% with metastases) in KPDC (+/−) and KPC (+/+) animals (*p<0.05, **p<0.01). (+/+) and (+/−) indicate WT and heterozygous deleted Dpc4, respectively.

(M) Immunoblots of representative KPDC and KPC primary PDA cell lysates from independent animals.

d, duodenum; ac, acinar cells. Scale bars, 50 μm. See also Table 1 and FIG. 8.

FIG. 2. Dpc4 status affects morphologic and cellular behaviors associated with metastasis

(A and B) Immunofluorescence of actin stress fibers (A) and surface E-cadherin (B) in representative KPC and KPDC PDA cells±TGFβ. Nuclei are counterstained with DAPI (blue). Scale bars, 50 μm.

(C) Cell migration of representative KPC and KPDC primary PDA cells from 3 independent experiments (mean±SEM; *p<0.0001).

(D and E) Representative images (D) and quantification (E) of KPDC and KPC cells after invasion through Matrigel (n=6 wells per cell line, *p<0.005).

(F-H) Representative images (F and G) and quantification (H) of pulmonary metastases after i.v. inoculation. Two different levels from three injected animals each were assessed (mean±SEM; *p<0.05).

FIG. 3. Runx3 promotes metastasis in murine PDA

(A) Runx3 qRT-PCR in KC preinvasive (Pre) and KPDC and KPC invasive PDA cells (n=4-5 each; mean±SEM; *p=0.05).

(B) Runx3 immunoblots in representative KPDC and KPC PDA cells. Actin, loading control.

(C) Runx3 expression in normal pancreas. Arrows, lymphocytes; arrowheads, duct; ac, acini; is, islet.

(D) Runx3 expression in undifferentiated region of invasive KPC PDA (arrows). Arrowhead, PanIN-1A.

(E) Runx3 expression in sarcomatoid region of invasive KPC PDA (arrows).

(F and G) Absence of Runx3 in carcinoma cells of two different KPDC PDA (arrowheads).

(H) PanIN in KPC animals lack Runx3 expression (arrowheads).

(I) Runx3 expression (arrows) in KPC liver metastases (outline) from primary PDA in panel (E); adjacent hepatocytes (h) lack Runx3 (arrowheads).

(J) Runx3 expression (arrows) in region of primary PDA from KPDC animal that developed metastases.

(K) Runx3 expression (arrows) in KPDC liver metastasis from primary PDA in (J) and lack of Runx3 (arrowheads) in adjacent hepatocytes (h). n, area of necrosis.

(L) Runx3 expression in KPDC and KPC primary tumors. Each point represents mean of three hpf from an independent animal (*p<0.05).

(M) Cell migration±TGFβ in representative KPDC cells with (Flag-Runx3) or without (Flag) Runx3 overexpression, and in, KPC cells with (shRunx3) or without (Scr) Runx3 depletion. Data represent three independent experiments (mean±SEM; *p<0.01).

(N and O) Representative lung sections (N) and quantification (O) of pulmonary metastases in mice injected with control (Flag) or Runx3-overexpressing (Flag-Runx3) KPDC#1 cells. Two different histological levels from a total of three injected animals for each condition were assessed (mean±SEM; *p<0.005).

(P and Q) Representative lung sections (P) and quantification (Q) of pulmonary metastases in mice injected with control (Scr) or Runx3-depleted (shRunx3) KPC#1 cells. Two different histological levels from a total of three injected animals each were assessed (mean±SEM; p=0.21). (Scale bars, 50 μm)

FIG. 4. Runx3 stimulates expression of pro-metastatic ECM components

(A) Immunoblots for Spp1 in KPDC and KPC primary PDA cells.

(B) Immunoblots for Spp1 in control (−) and Runx3-overexpressing (+) KPDC cells.

(C) Immunoblots for Spp1 in control (−) and Runx3-knockdown (+) KPC cells.

(D) KPDC and KPC primary carcinoma cell migration under the indicated conditions, representative of 3 independent experiments (mean±SEM; *p<0.001).

(E) Circulating Spp1 in KPC and KPDC animals with (+) or without (−) metastatic disease. Points represents the mean of duplicate measurements from mice (*p<0.005).

(F) Immunoblots for Col6a1 in independent KPDC and KPC primary PDA cell preparations. (Loading control was the same as in (A)).

(G) Immunoblots for Col6a1 in control (Flag) and Runx3-overexpressing (Flag-Runx3) KPDC cells. (Loading control was the same as in (B)).

(H) Immunoblots for Col6a1 in control (−, Scr) and Runx3-depleted (+, shRunx3) KPC cells. (Loading control was the same as in (C)).

(I) Col6a1 immunoblots of conditioned media from KPC and KPDC cell lines. Tenascin C, loading control.

(J) KPDC and KPC primary carcinoma cell migration+/−Col6a1 overexpression (Col6a1) or depletion (shCol6a1), respectively. Mean±SEM of 3 independent experiments (*p<0.0001).

(K) Representative lung sections from mice injected i.v. with either depleted or overexpressed Col6a1 in KPC and KPDC cells, respectively (n=2 cell lines and 3 animals for each condition).

(L) Quantification of metastatic pulmonary tumor burden from assays in (K). Two different histological levels from a total of three injected animals were assessed (mean±SEM; *p<0.05).

FIG. 5. Runx3 inhibits proliferation in invasive PDA cells

(A and B) Ki-67 expression in autochthonous (A) KPDC and (B) KPC PDA.

(C and D) Cleaved caspase 3 in autochthonous (C) KPDC and (D) KPC PDA.

(E) Proliferation in autochthonous KPDC and KPC tumors (mean±SEM, *p<0.05).

(F) Proliferation of purified PDA cells±TGFβ in vitro (n=3, mean±SEM; *p<0.001).

(G) Proliferation of control (Scr) and Runx3-knockdown (shRunx3) purified primary KPC cells in vitro (n=3, mean±SEM; *p<0.001).

(H) Proliferation of control (Flag) and Runx3-overexpressing (Fl-Runx3) purified KPDC cells in vitro (n=3, mean±SEM; *p<0.001).

(I) Immunoblots for p21 in control and Runx3-overexpressing KPDC cells and control and Runx3-depleted KPC cells. Fold changes were quantified by densitometry and normalized to actin.

Scale bars, 50 μm.

FIG. 6. Dpc4 and Runx3 coordinately regulate metastatic behavior in PDA

(A) Spontaneous focal loss of Dpc4 (left) in representative autochthonous KPDC PDA correlates with acquired Runx3 expression (right). Yellow boxes and arrowheads indicate areas of focal Dpc4 loss and acquired Runx3 expression in tumor epithelia; red boxes and arrows indicate regions of Dpc4 retention and undetectable Runx3.

(B) Liver metastases (asterisks and dotted outlines) from KPDC mice reveal spontaneous loss of Dpc4 and corresponding increases in Runx3 expression (arrowheads). Note that hepatocytes (h) retain Dpc4 and do not express Runx3.

(C and D) Quantification of Dpc4 and Runx3 IHC in (C) glandular structures in primary KPDC tumors and (D) liver metastases from KPDC mice. Runx3-low structures/metastases are represented by open bars and Runx3-high by black bars. Fisher's exact test revealed a significant correlation between Dpc4 loss and Runx3 expression (**p<0.0005, *p<0.005).

(E) Immunofluorescence of actin stress fibers (upper panels) and surface E-cadherin (lower panels) in KPDDC cells±TGFβ. Nuclei are counterstained with DAPI (blue).

(F) Metastatic potential (1) plotted as fraction of mice exhibiting metastases (red, ±95% confidence interval) and relative Runx3 protein levels (black outlined bars; mean±SEM) as a function of Dpc4 status (green).

(G) Model for the regulation and role of Runx3 in proliferation and metastasis of PDA. Runx3 levels are influenced by Dpc4 status in a biphasic manner and increase only after LOH of Trp53 in the context of point-mutant Trp53. Runx3 expression in turn stimulates synthesis and secretion of proteins that promote migration and metastatic niche preparation, while simultaneously inhibiting proliferation. (Scale bars, 50 μm)

FIG. 7. RUNX3 promotes metastasis in human PDA

(A) Immunoblots for RUNX3 in human PDA lines.

(B) Migration of control (Flag) and RUNX3-overexpressing (Flag-RUNX3) MiaPaCa-2 cells±TGFβ (n=3, mean±SEM, *p<0.05, **p<0.01).

(C) Migration of control (Scr) or RUNX3-knockdown (shRUNX3) Panc-1 and CFPAC-1 cells±TGFβ (n=3, mean±SEM, *p<0.01).

(D) Representative lung sections from NOD/SCID mice injected with control (Flag) or RUNX3-overexpressing (Flag-RUNX3) MiaPaCa-2 cells. Arrows, metastases.

(E) Quantified metastatic pulmonary tumor burden from assays in (D). Two histological levels from three injected animals were assessed (mean±SEM; *p<0.05).

(F) Livers in vivo (left) and ex vivo (right) from NOD/SCID animals injected with control (Scr) or RUNX3-knockdown (shRUNX3) Panc-1 cells. Arrows, metastases.

(G) Kaplan-Meier survival of patients after resection of a primary pancreas cancer. RUNX3 IHC of the primary tumor was used to stratify patients into high (score≥2; n=52) or low (score<2; n=36) populations; median survivals were 395 and 776 days, respectively (Wilcoxon p<0.018).

(H) ICGC gene array data for SPP1 and COL6A1 expression in PDA patients who experienced distant (D; n=39) vs. local (L; n=8) relapse after surgery (*p<0.001; p=0.14, COL6A1 comparison).

(I) Median survivals in patients (n=24) who received adjuvant systemic treatment with or without local radiation therapy as a function of RUNX3 status (low, score≤2; high, score>2).

(J) RUNX3 and DPC4 levels coordinately help inform clinical decision-making regarding potential therapeutic strategies and regimens for pancreas cancer, such as resectable PDA.

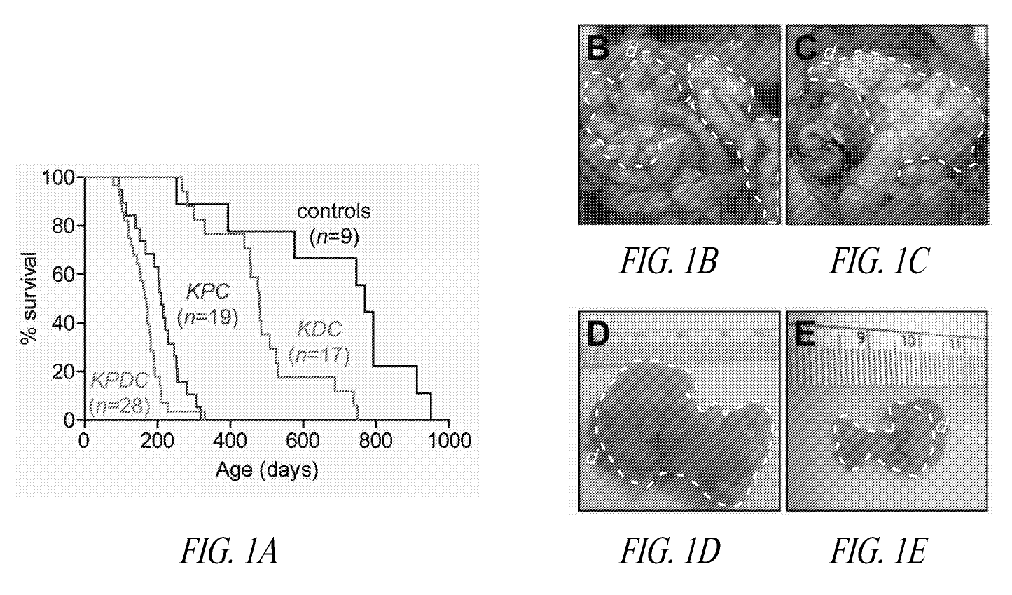

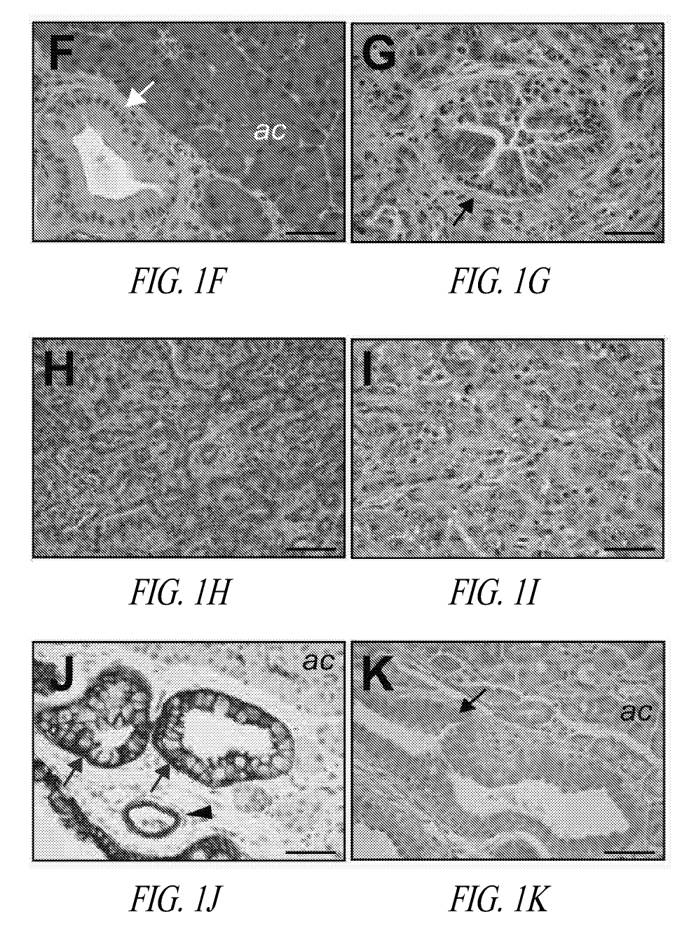

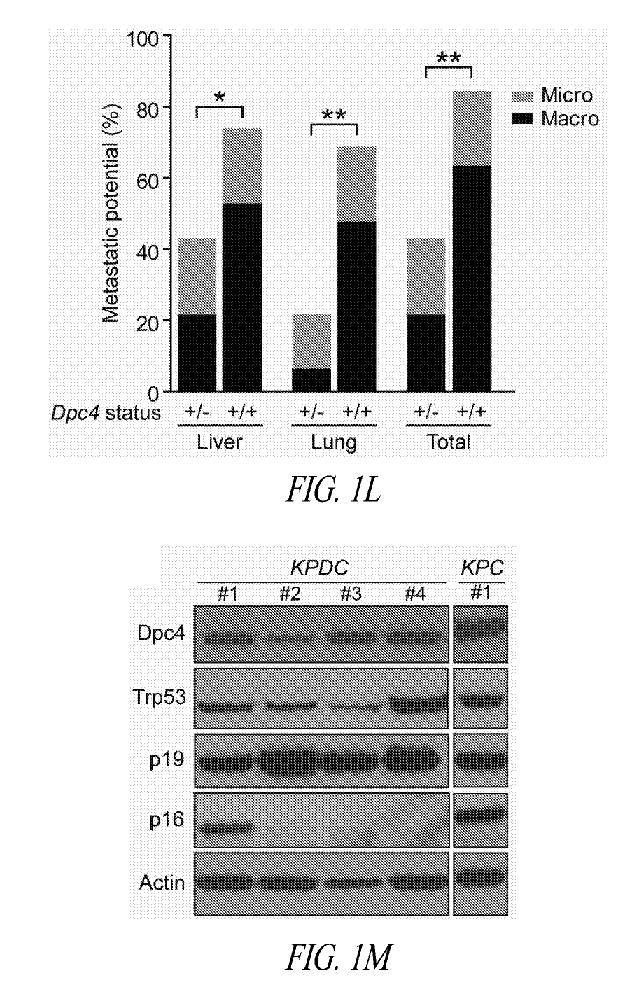

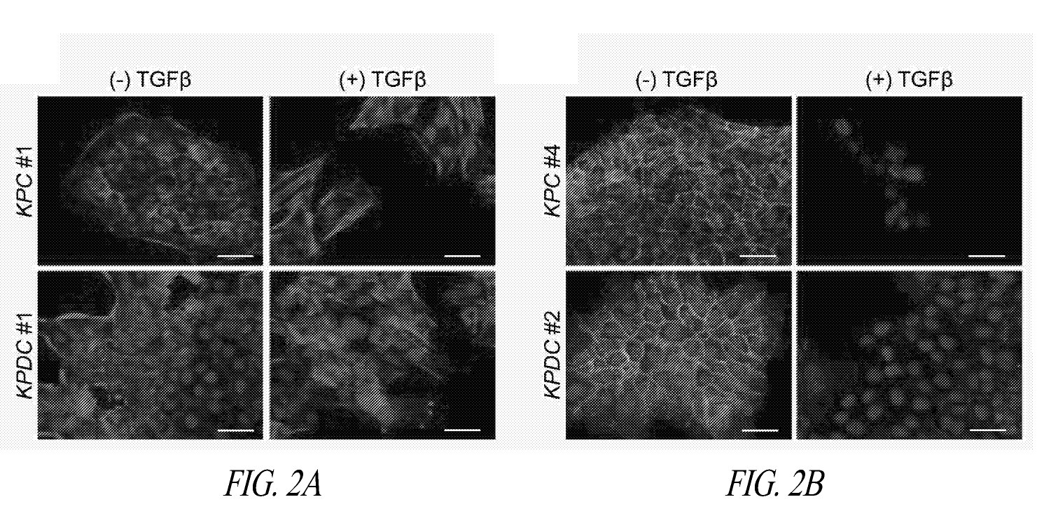

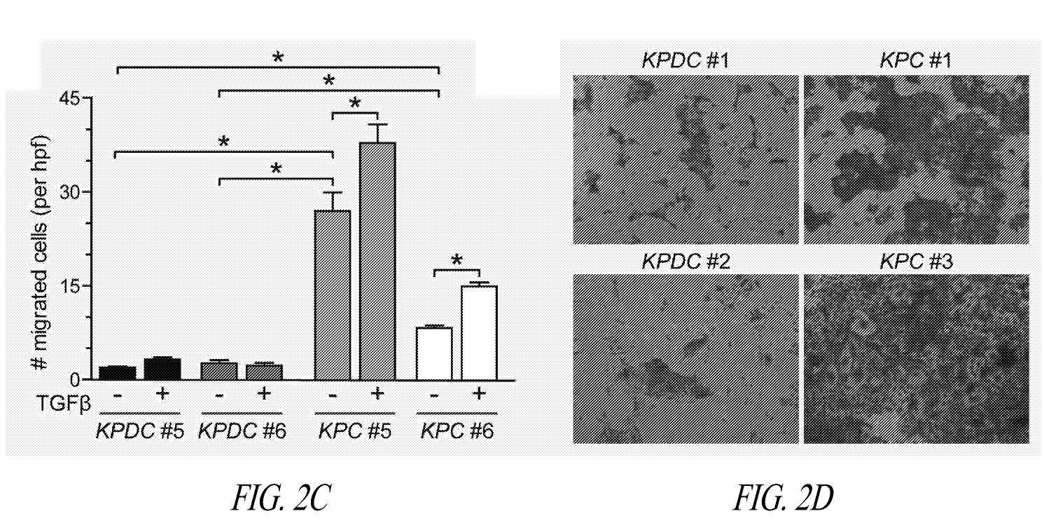

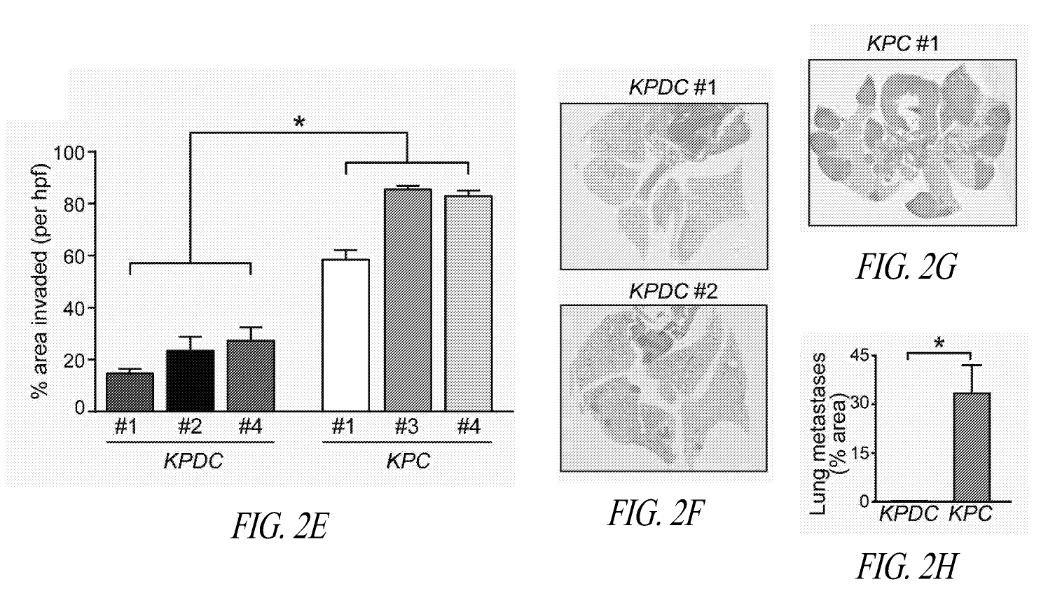

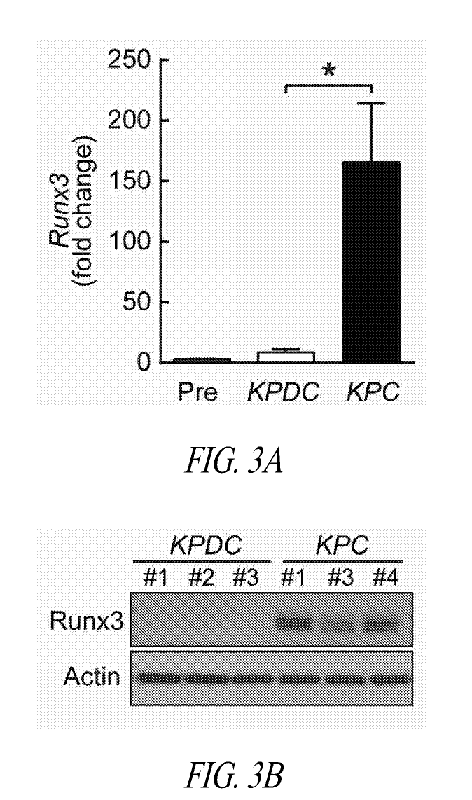

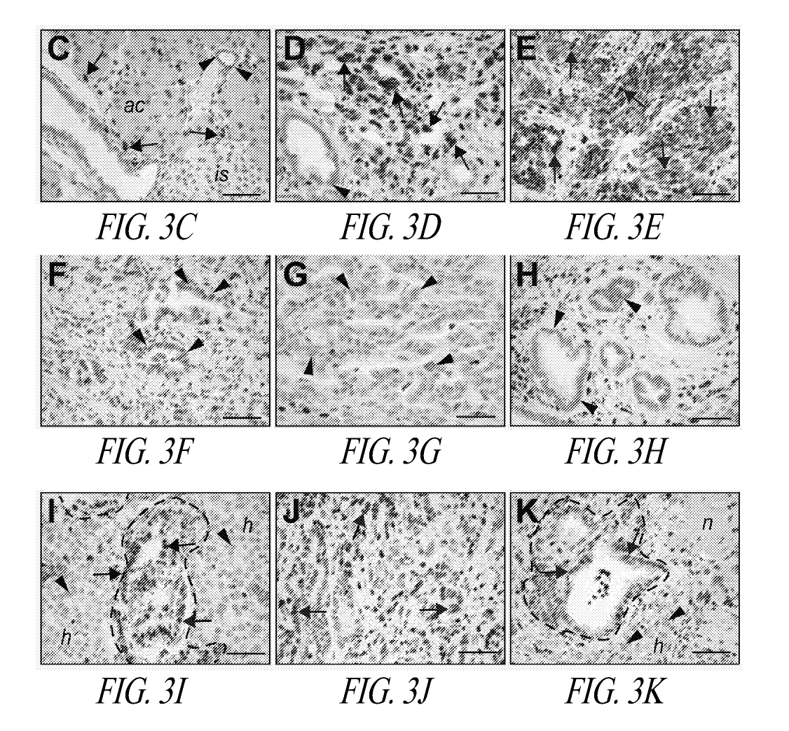

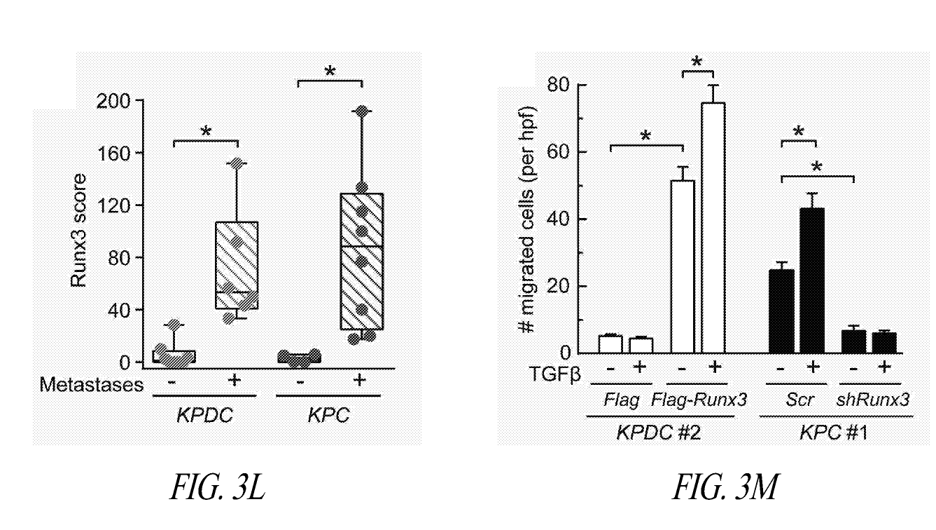

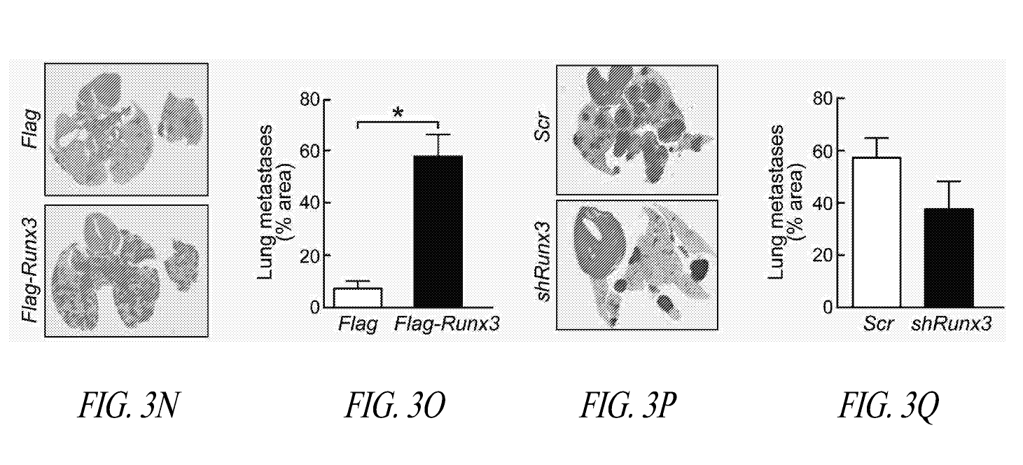

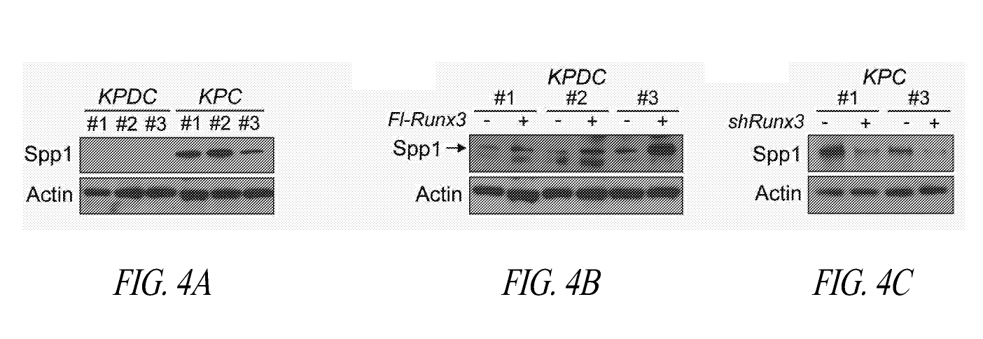

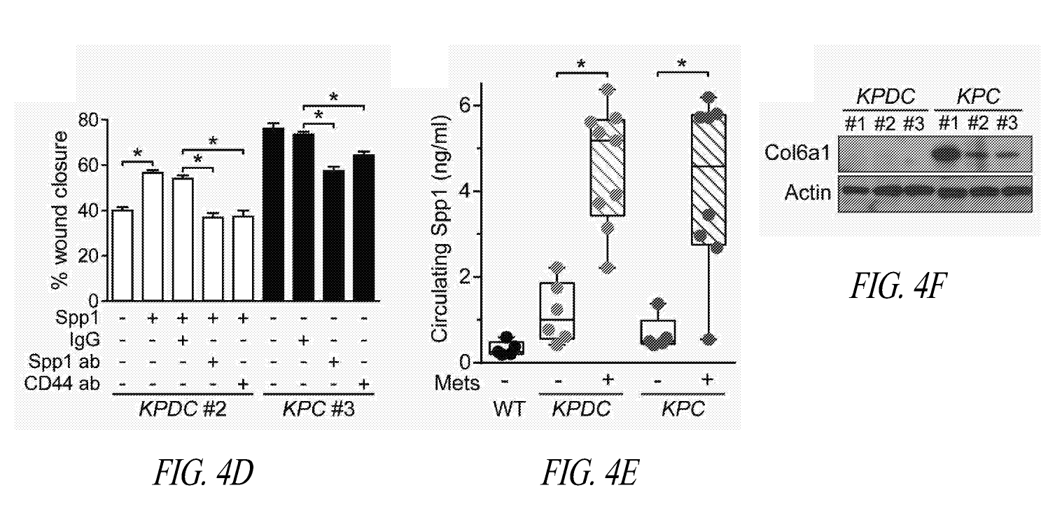

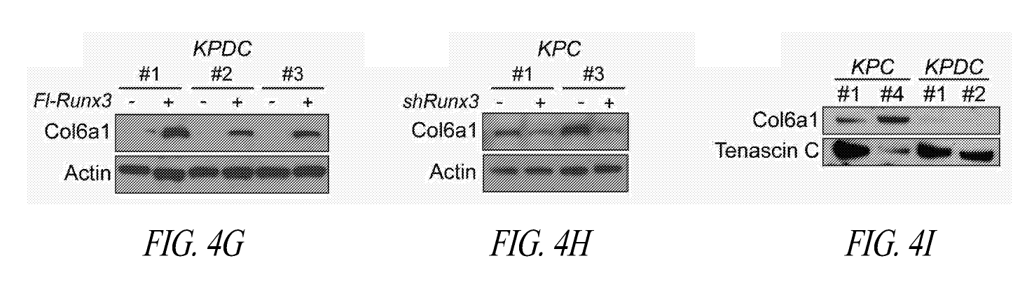

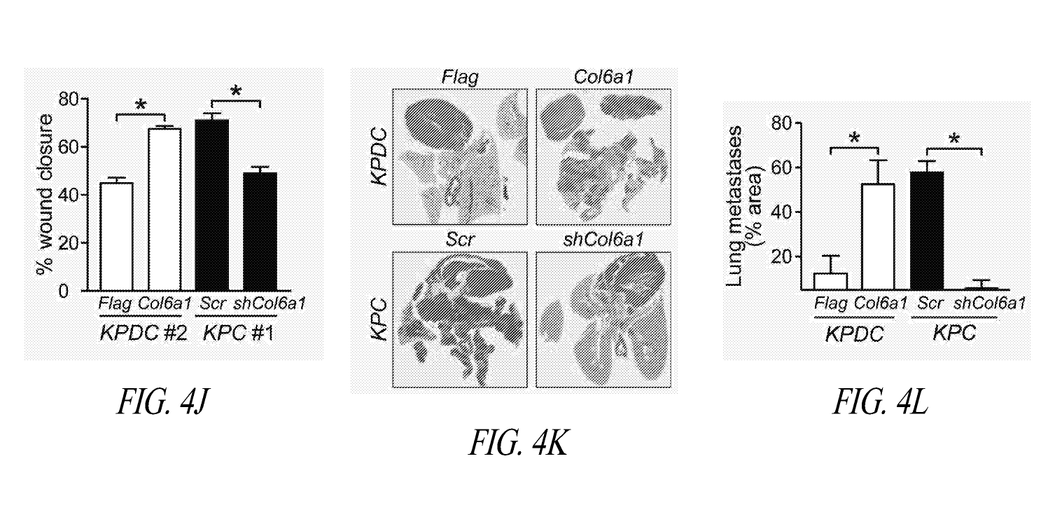

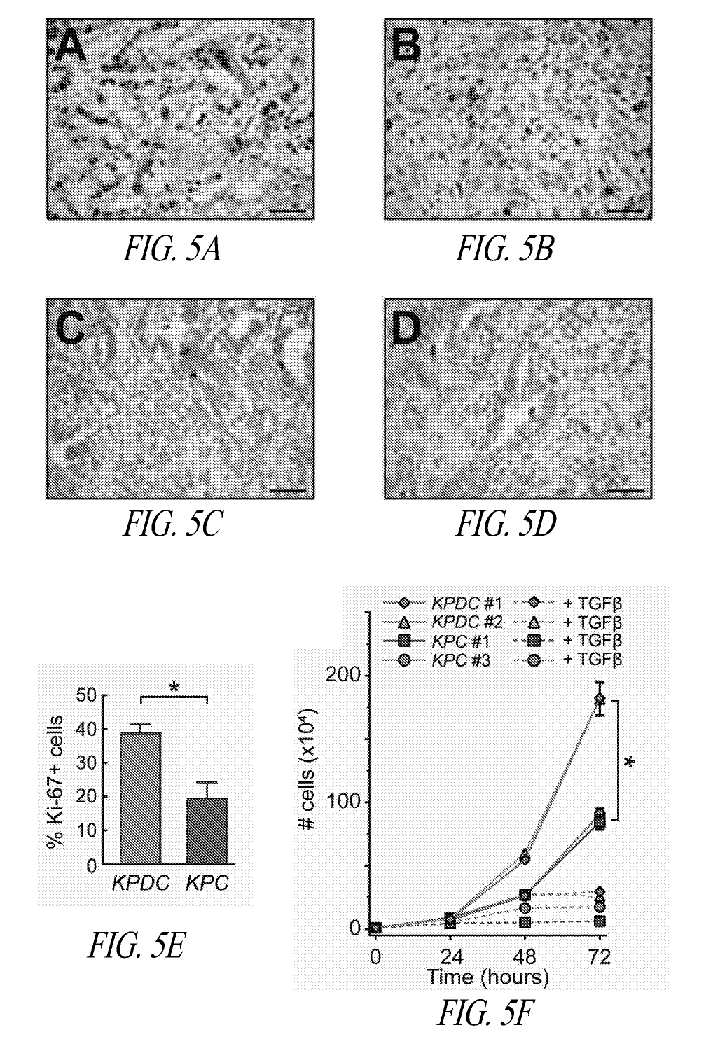

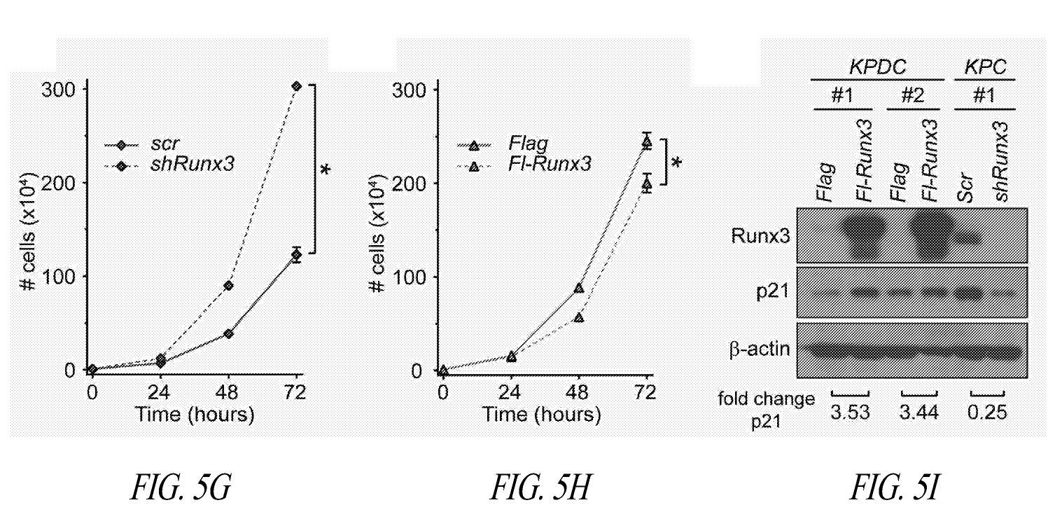

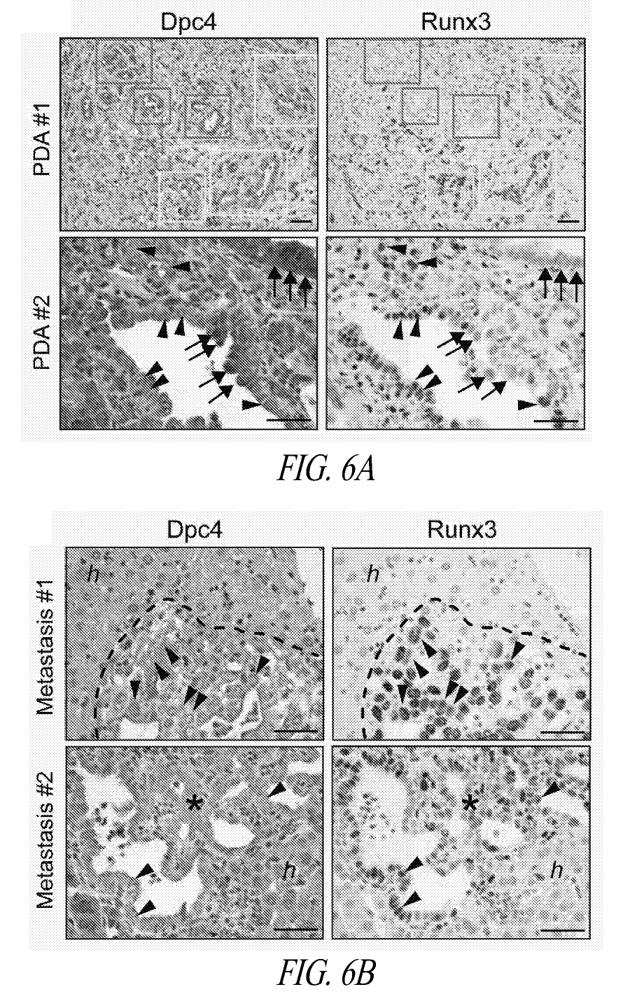

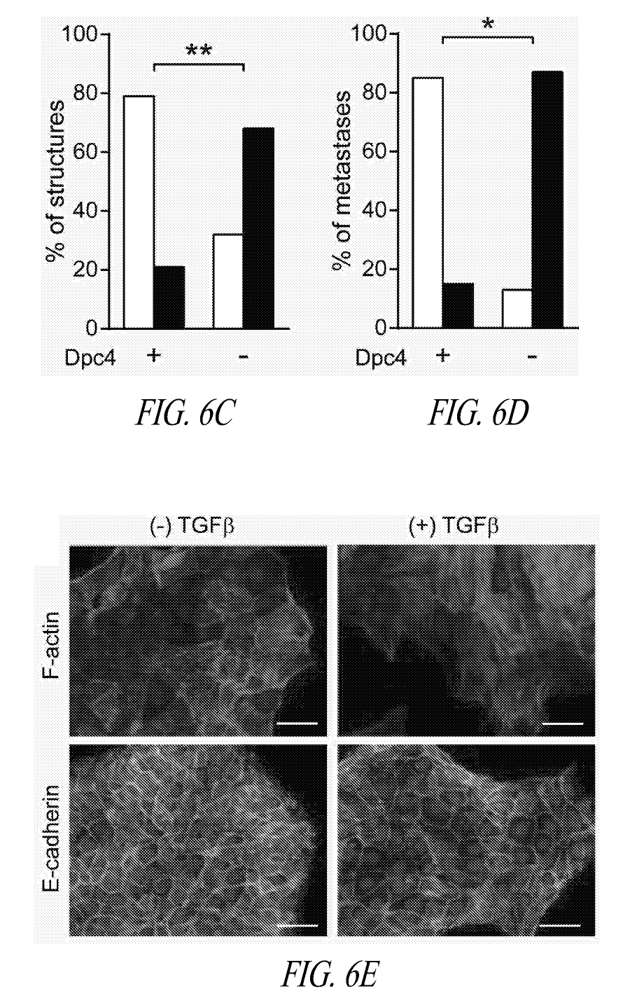

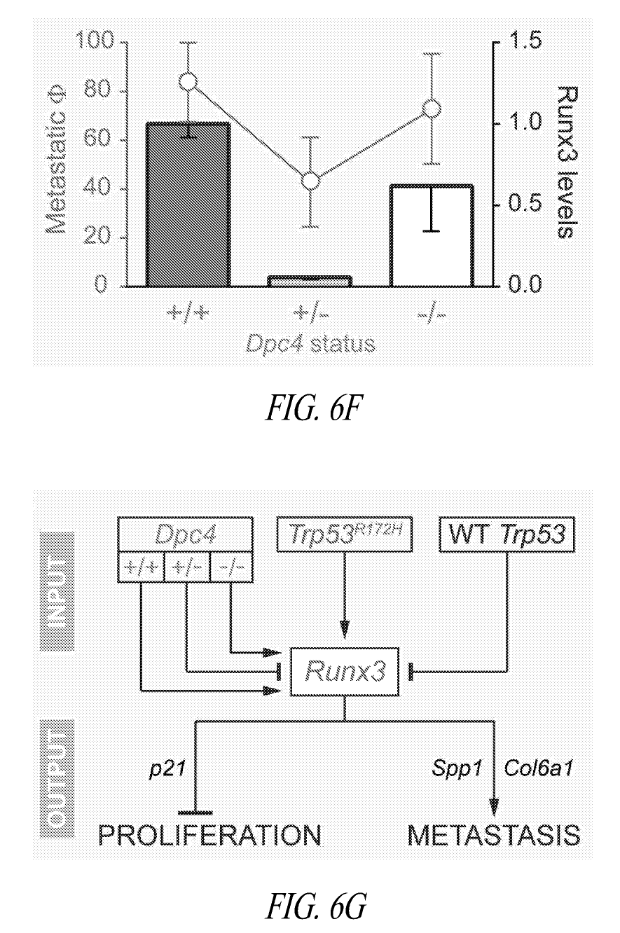

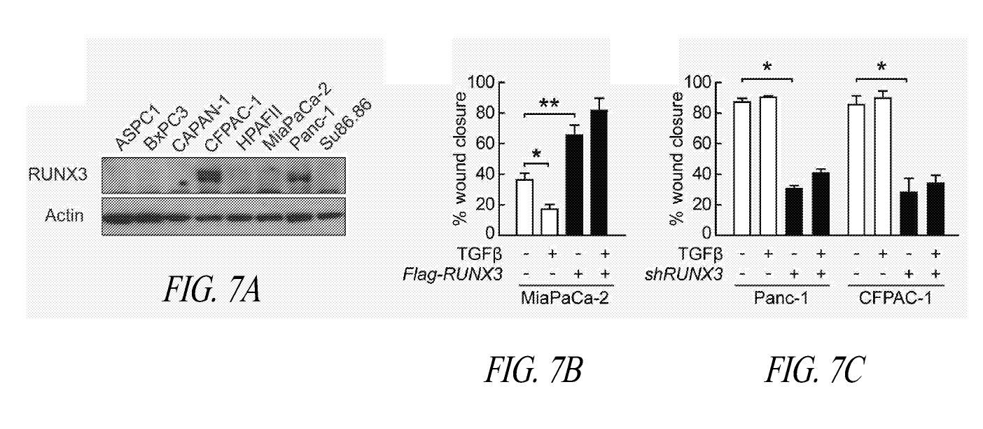

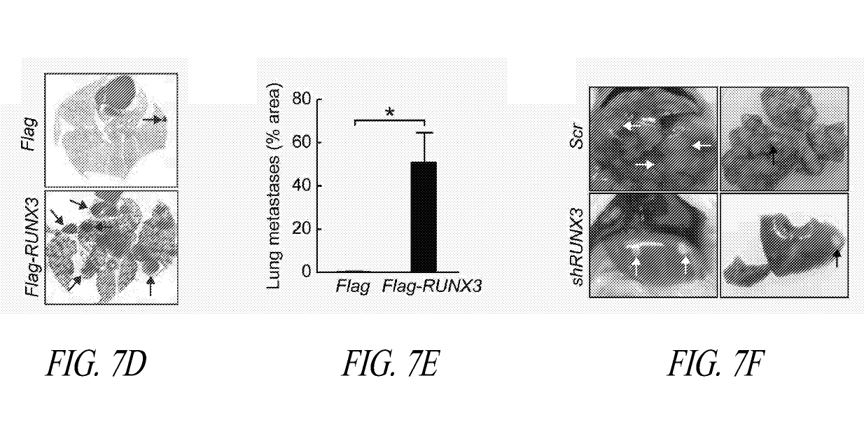

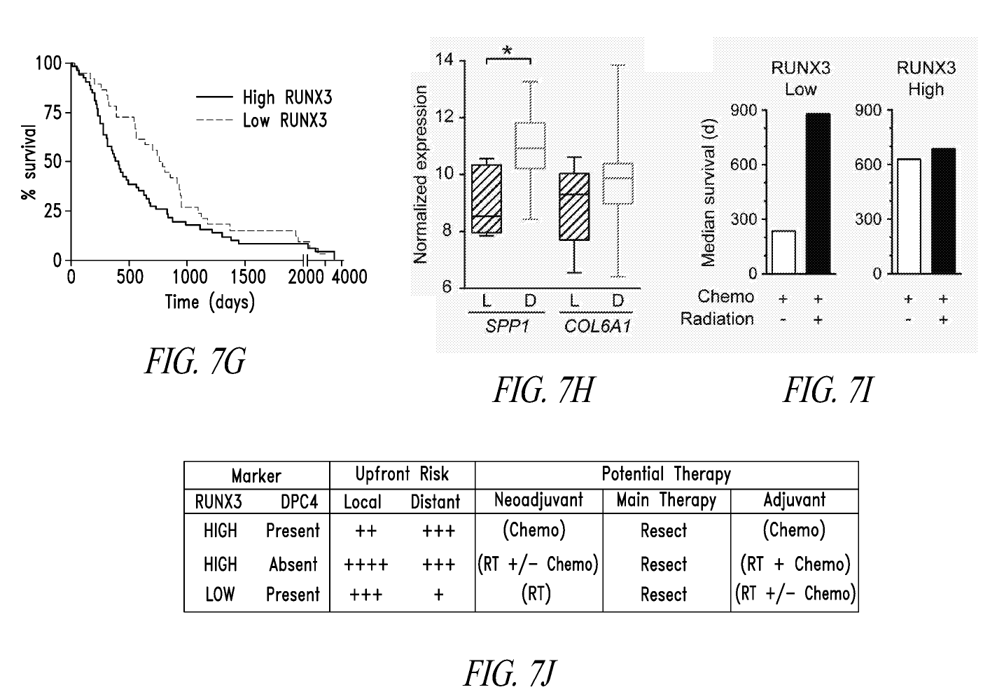

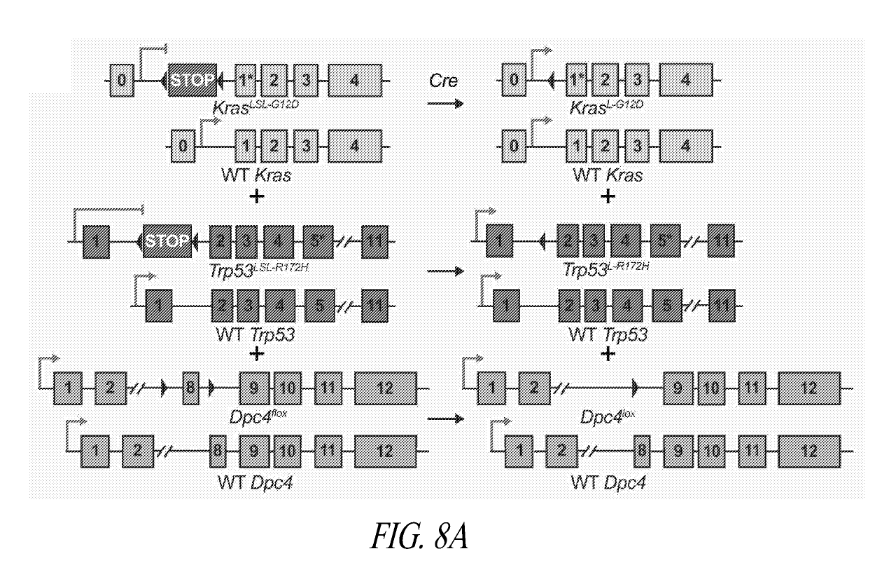

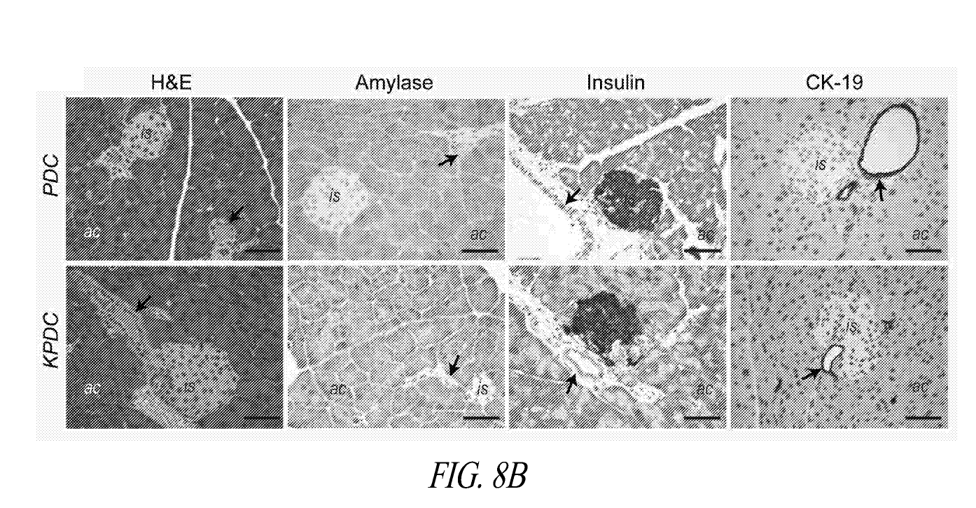

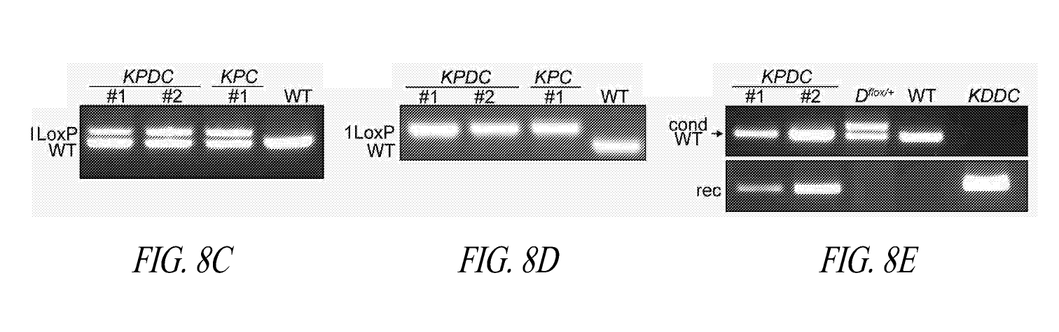

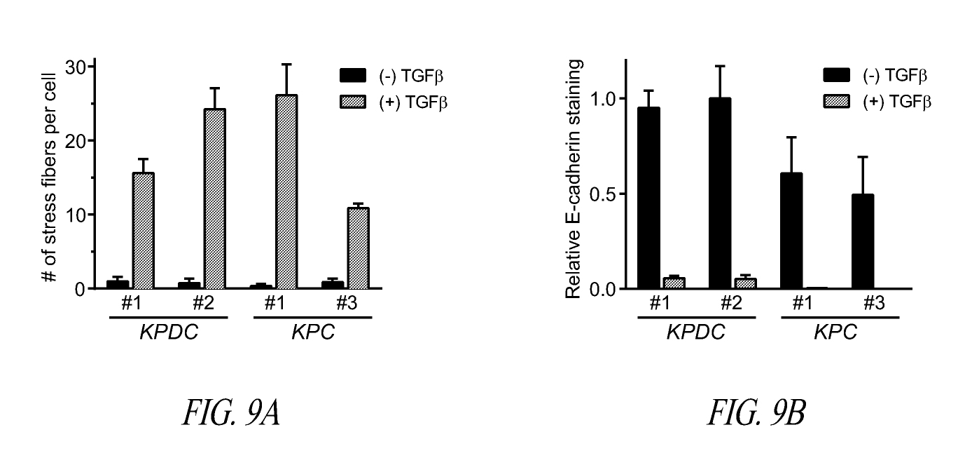

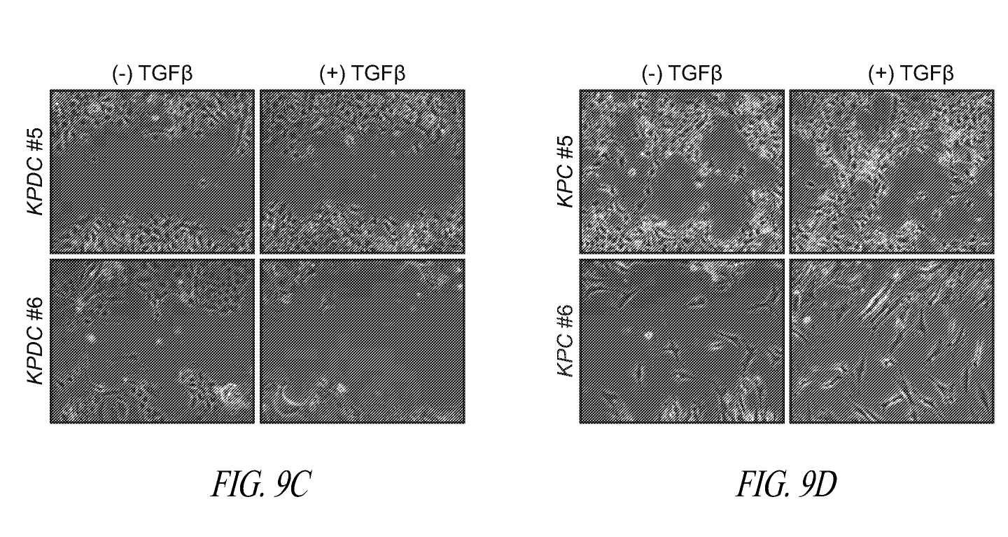

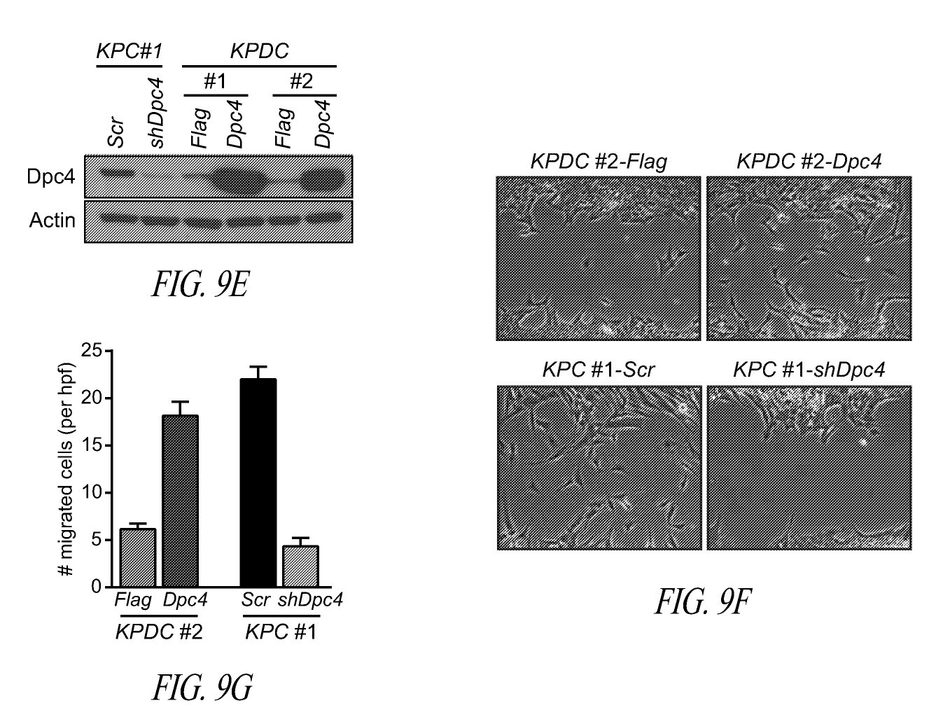

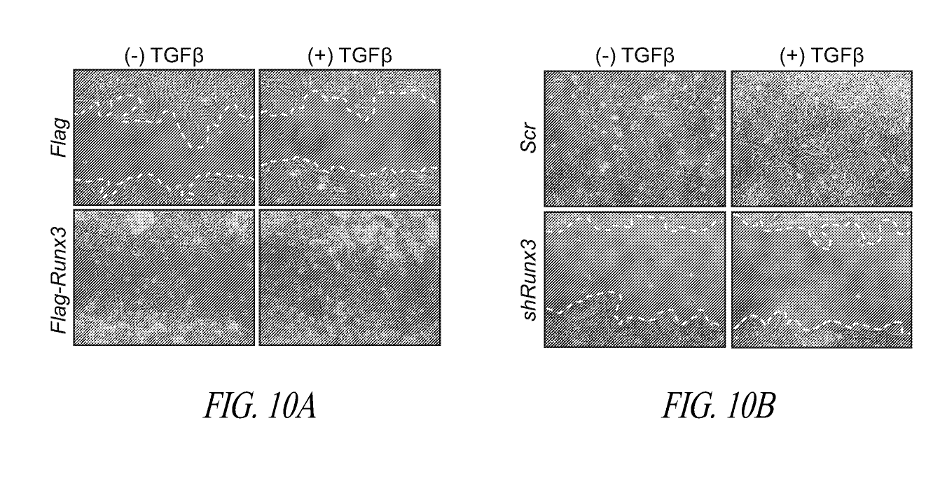

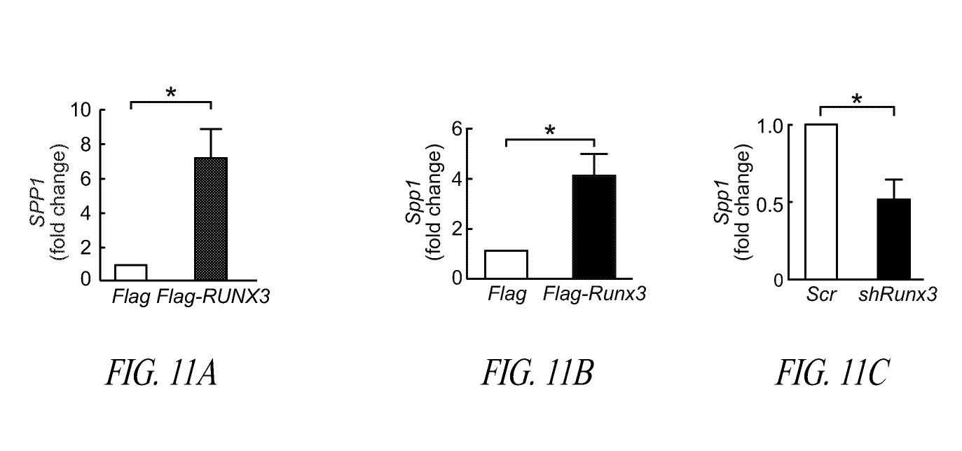

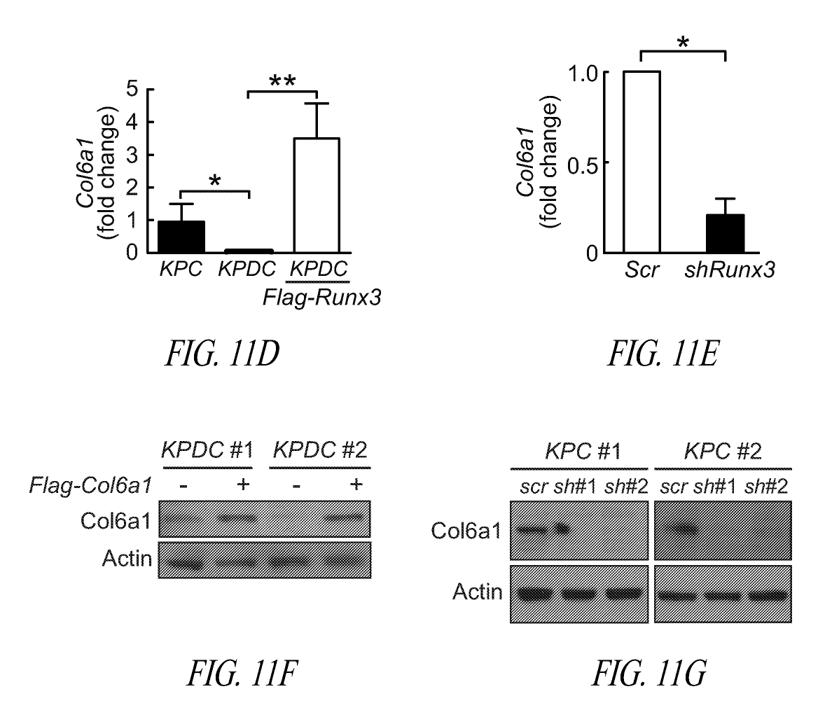

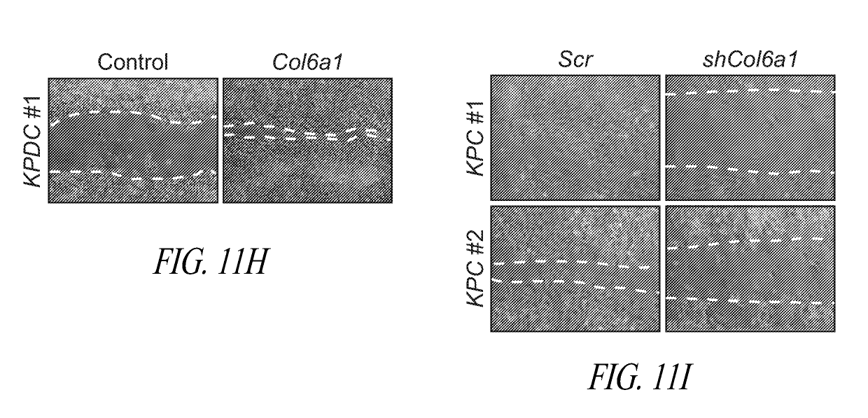

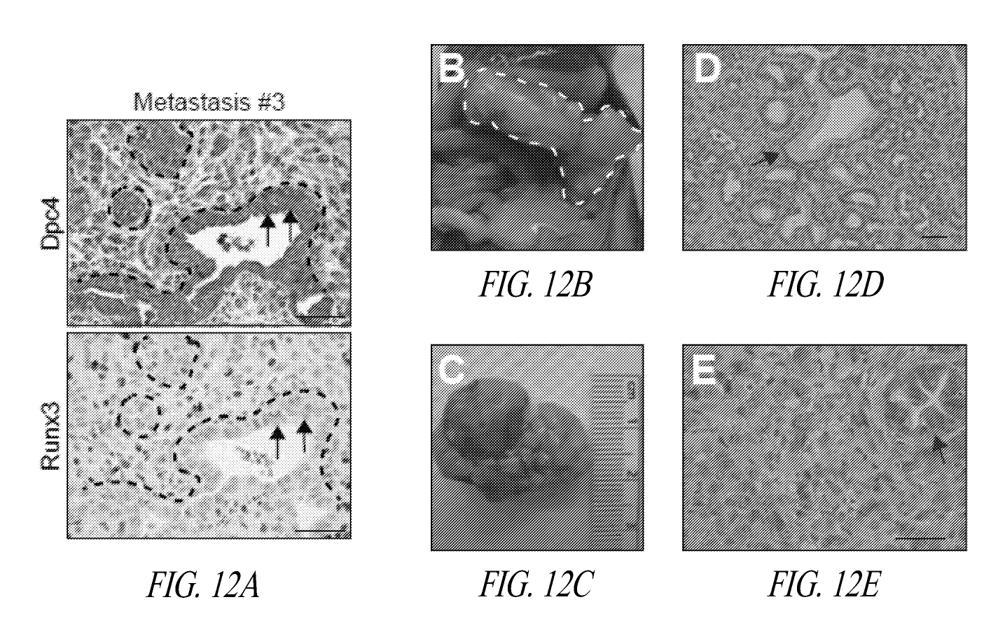

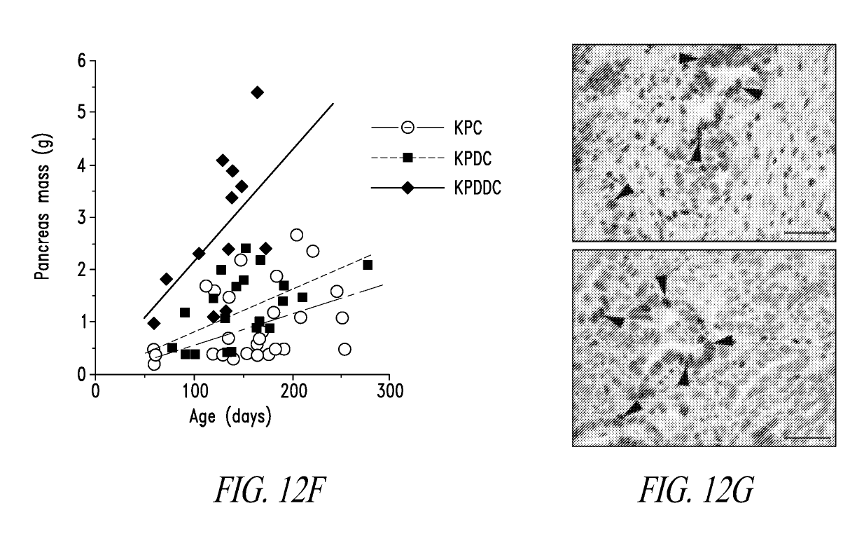

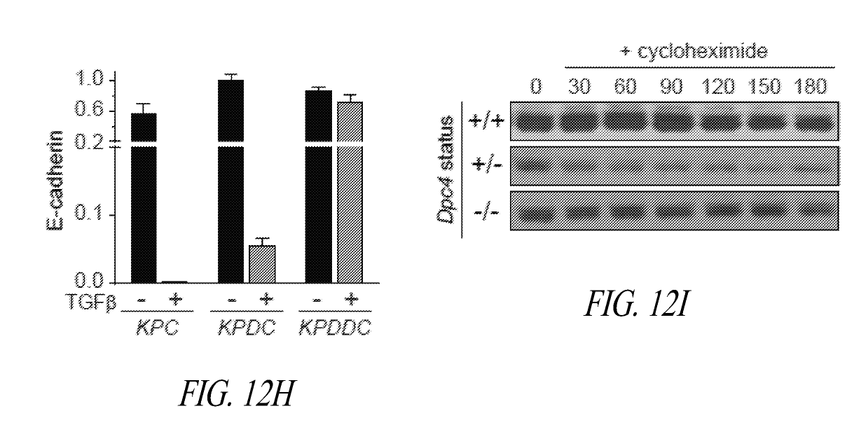

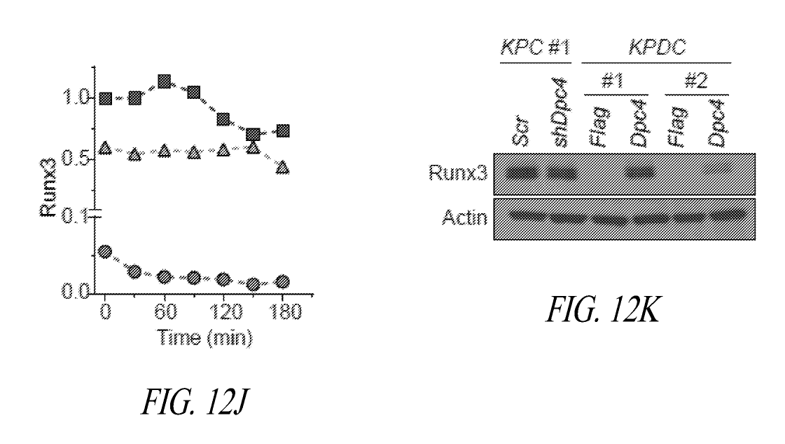

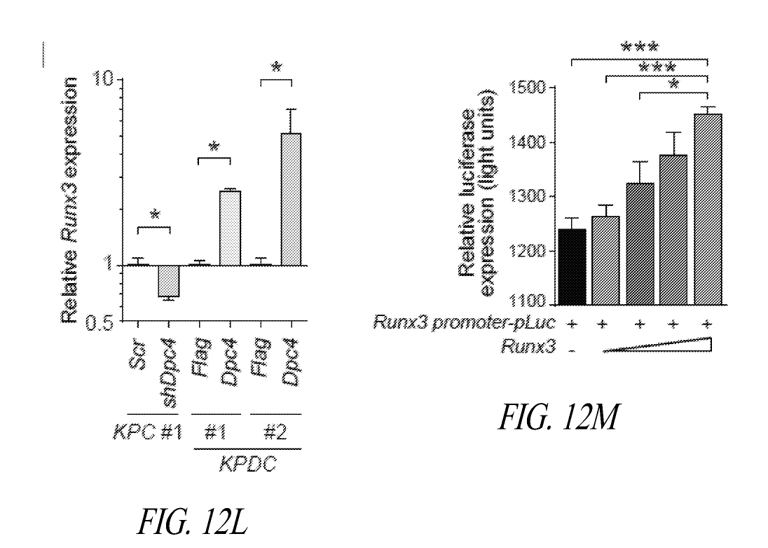

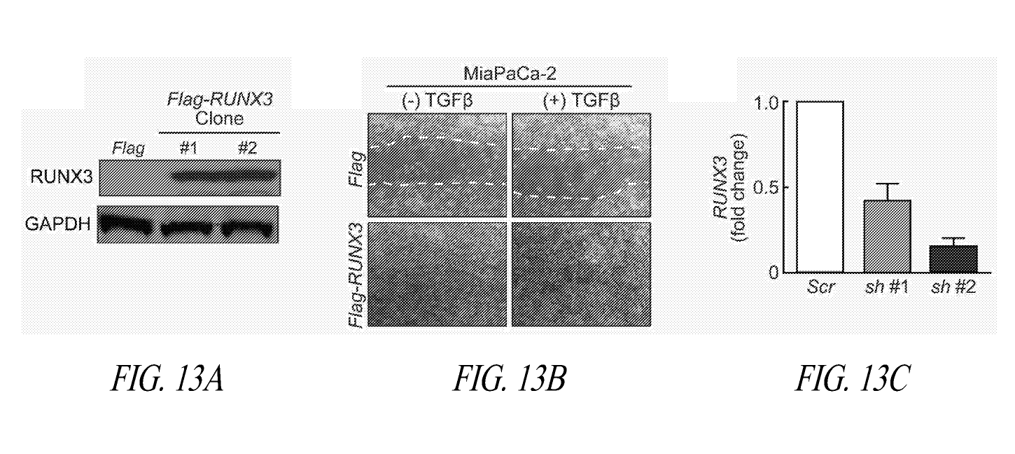

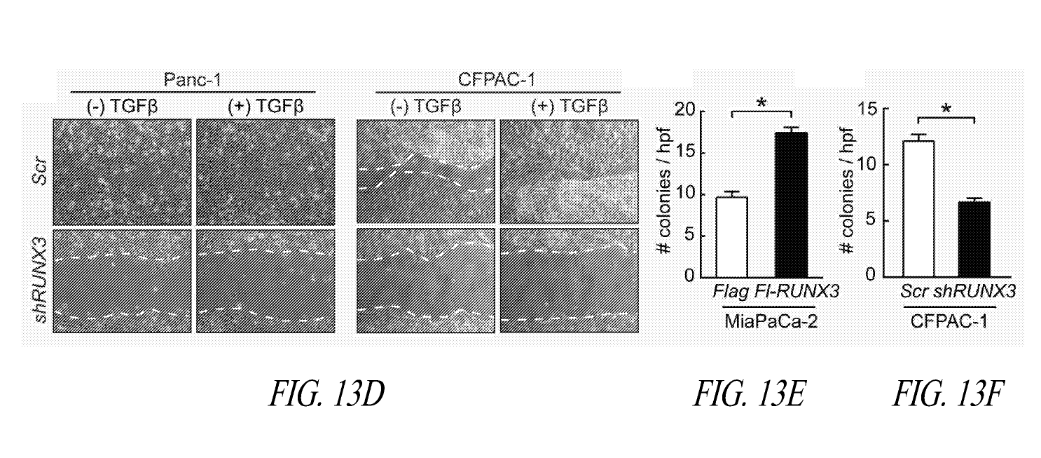

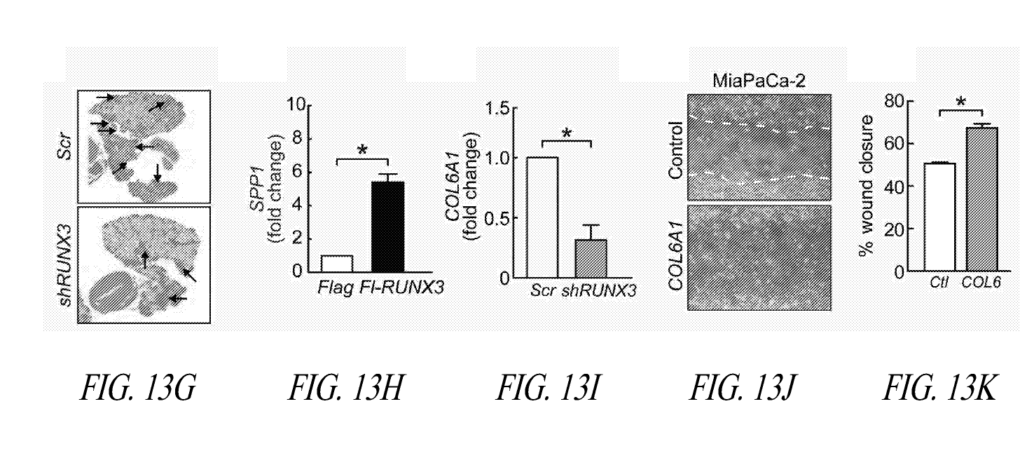

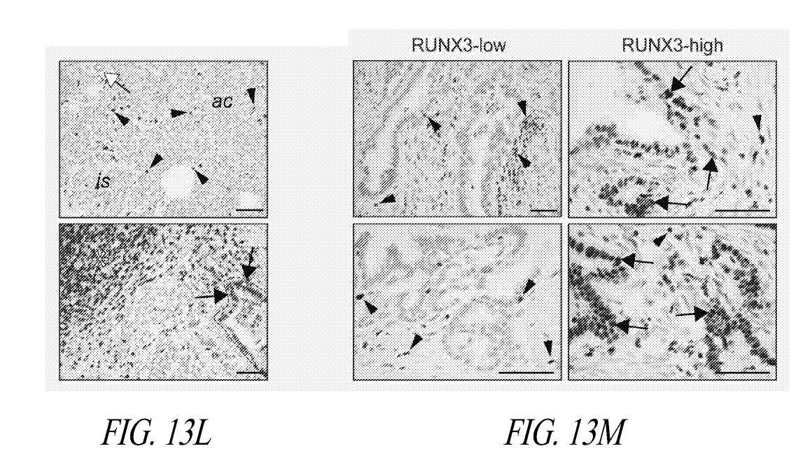

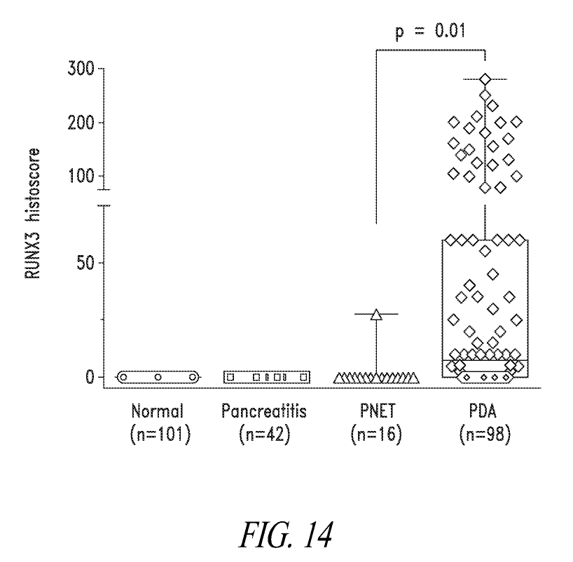

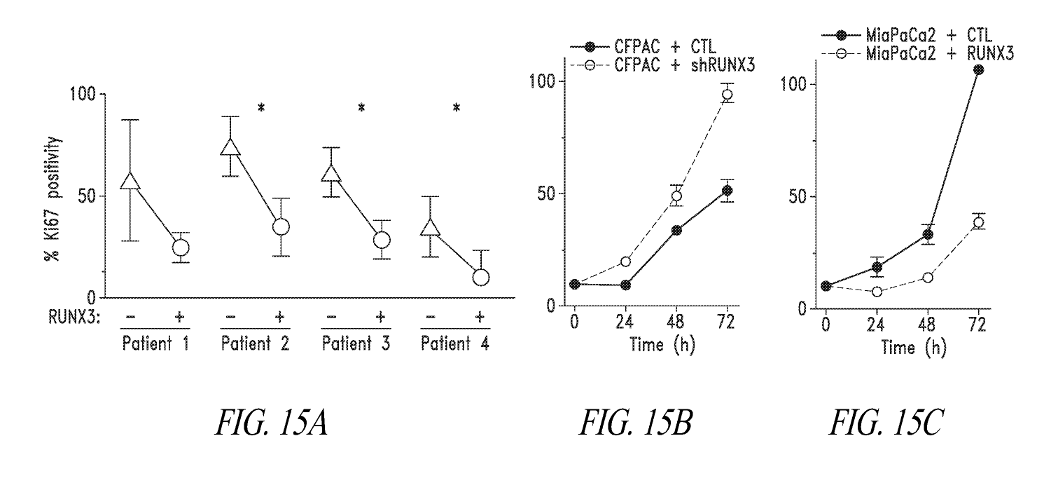

FIG. 8 (related to FIG. 1). Targeting endogenous Kras^{G12D} and Trp53^{R172H} expression with Dpc4 deletion to the murine pancreas

(A) Activation of Kras^{LSL-G12D} and Trp53^{LSL-R172H} and deletion of Dpc4^{flox} endogenous alleles upon pancreas-specific exposure to Cre recombinase (p48^{Cre}) in quadruple mutant Kras^{LSL-G12D/+}; Trp53^{LSL-R172H/+}; Dpc4^{flox/+}; p48^{Cre/+} (KPDC) mice.

(B) Immunohistochemical detection of digestive enzymes (amylase), hormones (insulin) and surface markers (CK-19) in distinct compartments of the pancreas from Trp53^{LSL-R172H/+}; Dpc4^{flox+}; p48^{Cre/+} (PDC) and young KPDC mice. is, islets; ac, acini; arrow, ducts. Scale bars, 50 μm.

(C) Evidence of recombined Kras^{L-G12D} (1LoxP, upper band) and wild type (WT) Kras (lower band) alleles in primary cells from KPDC and KPC PDA.

(D) Evidence of recombined Trp53^{L-R172H} allele (1LoxP, upper band) and uniform loss of WT Trp53 (lower band) in primary carcinoma cells from KPDC and KPC animals.

(E) Recombination (rec) of conditional (cond) Dpc4 allele and retention of WT allele in all KPDC primary carcinoma cell lines examined. WT, D^{flox/+} and KDDC (D^{flox/flox}) represent controls for various alleles.

FIG. 9 (related to FIG. 2). Dpc4 status affects morphological and cellular behaviors associated with metastasis

(A) Stress fiber formation in KPDC and KPC primary PDA cells in the absence or presence of TGFβ.

(B) Surface E-cadherin in KPDC and KPC primary PDA cells in the absence or presence of TGFβ.

(C and D) Representative images of wound migration in KPDC (C) and KPC

(D) cells in the absence or presence of TGFβ.

(E) Immunoblots for Dpc4 in control (Scr) and Dpc4-depleted KPC cells and control (Flag) and Dpc4-overexpressing KPDC cells.

(F) Representative images of wound migration in KPDC-Flag and KPDC-Dpc4 (upper panels) and KPC-Scr and KPC-shDpc4 (lower panels).

(G) Cell migration of KPDC PDA cells with (Dpc4) or without (Flag) Dpc4 overexpression and of KPC cells with (shDpc4) or without (Scr) Dpc4 depletion. Data represent average number of migrated cells in 4 high powered fields (hpf) across 3 independent experiments.

FIG. 10 (related to FIG. 3). Runx3 localizes to the nucleus and promotes metastasis in murine PDA

(A) Wound migration assays of Flag and Flag-Runx3 transfected KPDC cells in the absence or presence of TGFβ.

(B) Wound migration assays of Scrambled (Scr) and shRunx3 transfected KPC cells in the absence or presence of TGFβ.

FIG. 11 (related to FIG. 4). Runx3 promotes metastasis via modulation of ECM components

(A) Human SPP1 promoter-luciferase assay in HEK293 cells transfected with Flag or Flag-RUNX3 (*p<0.05).

(B) qRT-PCR of Spp1 in Runx3-overexpressing KPDC cells (*p<0.005).

(C) qRT-PCR of Spp1 in KPC cells after Runx3 depletion (*p<0.05).

(D) qRT-PCR of relative Col6a1 transcript levels in KPC, KPDC and KPDC-Flag-Runx3 carcinoma cells normalized to transcript levels in preinvasive KC cells (mean±SEM; n=3 cell lines for each genotype; *p<0.05, **p<0.01).

(E) qRT-PCR analysis of Col6a1 in KPC cells after Runx3 depletion (*p<0.05).

(F) Stable Col6a1 expression in KPDC primary cells confirmed by immunoblot.

(G) Stable depletion of Col6a1 in KPC primary cells with independent shRNAs (sh#1 and sh#2). Depletion was confirmed by immunoblot with Col6a1-specific antibody.

(H) Cell migration of control and Col6a1-transfected KPDC cells.

(I) Cell migration of control (Scr) and shCol6a1-transfected KPC cells.

FIG. 12 (related to FIG. 6). Dpc4 status affects morphologic and cellular behaviors associated with metastasis

(A) Example of relatively infrequent retention of Dpc4 expression in a liver metastasis and correspondingly undetectable levels of Runx3 expression. Arrow, epithelial cells in a glandular structure.

(B and C) Gross pathology of pancreata from representative KPDDC mice. Dashed line, tumor.

(D) Precursor lesions (PanIN) in a KPDDC animal. Arrow, PanIN-1A. Scale bars, 50 μm.

(E) Region of undifferentiated PDA from a KPDDC animal. Arrow indicates what was a likely PanIN-3 prior to invasion. Scale bars, 50 μm.

(F) Autochthonous growth of KPC (n=30), KPDC (n=21) and KPDDC (n=12) primary PDA. Linear regression revealed significantly different growth rates for each genetic subtype (p<0.05).

(G) Runx3 expression in epithelia of a KPDDC primary tumor (upper panel) and lung metastasis (lower panel). Arrowheads, Runx3-positive tumor epithelial cells. Scale bars, 50 μm.

(H) Surface E-cadherin loss following TGFβ treatment in KPC, KPDC and KPDDC cell lines (n=2 each).

(I) Immunoblots for Runx3 in protein lysates from KPC#1 (+1+), KPDC#1 (+/−), and KPDDC#1 (−/−) cells incubated with cycloheximide for the indicated times in minutes.

(J) Runx3 levels after cycloheximide treatment in KPC#1 (blue squares), KPDC#1 (red circles) and KPDDC#1 (green triangles) quantified by densitometry from immunoblots in (K).

(K) Immunoblots of Runx3 in KPC and KPDC cells with knockdown and overexpression of Dpc4, respectively (*p<0.05).

(L) qRT-PCR of Runx3 transcript in KPC and KPDC cells with knockdown and overexpression of Dpc4, respectively (*p<0.05).

(M) Expression assays performed in Runx3-promoter-Luciferase-transfected HEK293T cells in the presence of increasing concentrations of exogenous Runx3 plasmid (6-500 ng) (*p<0.05; ***p<0.001).

FIG. 13 (related to FIG. 7). RUNX3 promotes metastasis in human PDA

(A) Expression of human Flag-RUNX3 in two MiaPaCa-2 cell clones (#1 and #2) was confirmed by immunoblotting. GAPDH, loading control.

(B) Representative images of cell migration assays of Flag- and Flag-RUNX3-transfected MiaPaCa-2 cells in the absence or presence of TGFβ.

(C) Efficiency of targeted RUNX3 depletion in Panc-1 cells with different shRNA constructs assessed by qRT-PCR.

(D) Representative images of wound migration assays of control (Scr) and shRUNX3-transfected CFPAC-1 and Panc-1 cells in the absence or presence of TGFβ.

(E-F) Anchorage-independent growth in soft agar of (E) control (Flag) or RUNX3-overexpressing (Flag-RUNX3) MiaPaCa-2 cells and (F) control (Scr) or RUNX3-knockdown (shRUNX3) CFPAC-1 cells (mean±SEM, *p<0.0001).

(G) Representative lung sections from NOD/SCID mice injected intravenously with control (Scr) or RUNX3-depleted (shRUNX3) Panc-1 cells. Arrows, metastases.

(H) SPP1 expression in Flag- and Flag-RUNX3 transfected MiaPaCa-2 cells (*p<0.001).

(I) COL6A1 expression in Scr and shRUNX3 transfected Panc-1 cells (*p<0.05). (J and K) Representative images (J) and quantification (K) of cell migration assays of control and COL6A1-transfected MiaPaCa-2 cells. Data represent percent recovery of scratch area from a minimum of 4 hpf across 3 independent experiments (mean±SEM, *p<0.0001).

(L) RUNX3 immunohistochemistry of control tissue from human PDA tissue microarray. Normal pancreas tissue (upper panel) adjacent to human PDA shows no detectable RUNX3 expression except in infiltrating leukocytes (arrowheads). Lymph nodes (lower panel, *) in patient PDA samples show high RUNX3 staining, serving as an internal positive control for comparison to RUNX3-positive tumor epithelial cells (arrows). Open arrow, terminal duct. is, islet; ac, acinar cells. Scale bars, 50 μm.

(M) Representative RUNX3 immunohistochemistry of human PDA showing examples of RUNX3-low (left panels; score<2+) and RUNX3-high (right panels; score>2+) samples from a tissue microarray. Arrows, RUNX3(+) tumor epithelial cells; arrowheads, RUNX3(+) infiltrating leukocytes. Scale bars, 50 μm.

FIG. 14 (related to FIGS. 3 and 13). RUNX3 is overexpressed in PDAs, but not in other human pancreas conditions.

Tissue samples representing normal (n=101), pancreatitis (n=42), pancreatic neuroendocrine tumor (PNET; n=16), and pancreatic ductal adenocarcinoma (PDA; n=98) conditions were assayed for RUNX3 expression. RUNX3 was not expressed in normal human pancreas or benign pancreatitis, rarely expressed in PNETs, and frequently overexpressed in PDAs (p=0.01).

FIG. 15 (related to FIG. 7). RUNX3 expression attenuates proliferation in human PDA.
(A) PDA samples from four patients were tested for Ki67 (a marker of proliferation). In regions of human PDA lacking (−) RUNX3 expression, proliferation was higher than in regions expressing (+) RUNX3.

(B) shRUNX3-transfected CFPAC-1 cells (dashed line) showed increased proliferation relative to control (solid line), as measured by Ki67 staining.

(C) Overexpression of RUNX3 in MiaPaCa2 cells decreased proliferation (dashed line) relative to control (solid line), as measured by Ki67 staining.

### DETAILED DESCRIPTION

The instant disclosure provides methods for diagnosing or detecting the potential of pancreas cancer cells to metastasize, to proliferate locally (tumor growth), or to have the ability to do both. In particular, various biomarkers examined individually and in various combinations will indicate what therapies, therapeutic regimens or combinations thereof (including neoadjuvant therapy, main therapy, and adjuvant therapy) will be the most beneficial to a subject at risk of developing pancreas cancer, having pancreas cancer, or at risk of pancreas cancer recurrence. Such biomarkers include DPC4, RUNX3, Col6a1 and Spp1.

The instant disclosure further provides methods for treating pancreatic cancer or for reducing the risk of pancreatic cancer (also called “pancreas cancer” herein) or pancreatic cancer metastasis by administering to a subject (e.g., having or requested to be screened for having the above-noted DPC4 and RUNX3 levels) a main therapy in combination with a neoadjuvant therapy, an adjuvant therapy or both, wherein the main therapy comprises tumor resection and: (a) the neoadjuvant therapy comprises a systemic therapy and an optional adjuvant therapy comprises a systemic therapy when the pancreas cancer cells from the subject are homozygous null for DPC4 and the pancreas cancer cells from the subject express RUNX3 at higher levels than normal cells from the subject; or (b) an optional neoadjuvant therapy comprises a radiotherapy and the adjuvant therapy comprises a radiotherapy when the pancreas cancer cells from the subject are heterozygous for DPC4, and the pancreas cancer cells from the subject express RUNX3 at levels similar to normal cells from the subject; or (c) an optional neoadjuvant therapy comprises a radiotherapy, chemotherapy or both, and the adjuvant therapy comprises a chemotherapy, radiotherapy, or both, when the pancreas cancer cells from the subject are homozygous null for DPC4/SMAD4, and the pancreas cancer cells from the subject express RUNX3 at higher levels than normal cells from the subject.

In certain embodiments, a systemic therapy comprises a chemotherapy, combined chemotherapies, biologic therapy, hormonal therapy, or any combination thereof. Exemplary chemotherapies include 5-fluorouracil, capecitabine, gemcitabine, bendamustine, cisplatin, irinotecan, paclitaxel, docetaxel, or any combination thereof. Representative biologic therapies include an antibody (e.g., cetuximab, trastuzumab, bevacizumab, ipilimumab, pembrolizumab, nivolumab, avelumab, or any combination thereof), a fusion protein (e.g., chimeric antigen receptor (CAR), such as used in adoptive immune thereapy comprising a T cell expressing an antigen specific CAR), a tyrosine kinase inhibitor, or any combination thereof. In any of these embodiments, the subject has early-stage pancreas cancer.

By way of background, the unusual ability of pancreatic ductal adenocarcinoma (PDA) to spread has spurred efforts to advance systemic neoadjuvant therapies and address at the outset the almost inevitable and frequently rapid appearance of metastases. This strategy is not without risks: beyond the potential toxicities looms the possibility that a previously contained tumor will breach the immediate confines of resectability. Indeed, a minority of early stage (I/II) or locally advanced (stage III) tumors are destined to remain local; these patients can often be managed for many months with no overt evidence of dissemination. In such patients, treating with chemotherapy first and delaying surgery may forfeit the rare window to operate. In a large meta-analysis of over 4,000 early stage pancreas cancer patients who received some form of neoadjuvant therapy, fully 30% became inoperable during the course of treatment (Gillen et al., PLoS Med. 7:e1000267, 2010). The ideal types, timing, duration and sequence of adjuvant chemotherapy and radiotherapy are also unclear, with the role of radiation being especially contentious (Katz et al., Oncologist 15:1205, 2010). Conflicting results in the setting of complex trial designs have confounded meaningful comparisons and precluded consensus on how best to incorporate distinct modalities into PDA management.

Taking the specific case of early-stage pancreas disease as an example, distinguishing those patients to treat with a neoadjuvant therapy from those patients needing an immediate tumor resection can be determined using the compositions and methods of this disclosure. For example, the instant disclosure provides that RUNX3 interpreted in the context of DPC4 status will help inform such clinical and therapeutic decisions. An exemplary method or decision tree of this disclosure for choosing a potential therapy or therapeutic regimen is provided in FIG. 7J. In certain embodiments, a given therapy can be tailored as appropriate for a particular patient depending on the DPC4 genotype, the level of RUNX3 expression, both, or in the context of other biomarkers. For example, high levels of RUNX3 increase the migratory and metastatic potential of both murine and human pancreas cancer cells (e.g., PDA) which, in the context of intact DPC4, defines the most likely cause of death. In certain embodiments, these patients would likely benefit most from initial systemic therapy followed by tumor resection given the comparatively lower risk for local tumor growth. In further embodiments, patients with resectable disease (e.g., pancreas tumors) and low RUNX3 expression level benefit less from systemic treatment up-front (i.e., neoadjuvant) and should instead be resected (or treated with a short course of directed radiation prior to resection, if needed). In still further embodiments, patients with high RUNX3 expression and complete loss of DPC4, an accepted therapeutic regimen sequence is inverted, wherein a short course of chemoradiation is administered to achieve some local control prior to systemic cytotoxics or chemotherapy.

By way of further background, but not wishing the bound by theory, patients with high RUNX3 and complete loss of DPC4 pose the greatest challenge since the use of a neoadjuvant therapy may allow for metastatic spread to occur and, therefore, may close the window for resecting a tumor with a positive prognosis. In related embodiments, a primary pancreas tumor of such patients receiving (or benefiting the most from) an inverted therapeutic regimen must be followed closely so as not to lose the opportunity for a definitive resection to, for example, minimize the risk of metastatic spread. Alternatively, in certain other related embodiments, systemic therapy can be combined with cell cycle inhibitors to provide treatment of occult disseminated disease while at the same time minimizing the risks of local tumor growth.

Familiarity with DPC4 and RUNX3 may be helpful to an understanding of the present disclosure. DPC4 (homozygously Deleted in Pancreatic Carcinoma, locus 4; also known as SMAD4) was first identified as a candidate tumor suppressor gene implicated in pancreatic tumorigenesis (Hahn et al., Science 271:350-353, 1996). DPC4 is located on chromosome 18q21.1, a region characterized by a high frequency of loss of heterozygosity (LOH) in pancreas carcinomas (Hahn et al., Cancer Res. 56:490, 1996). DPC4 is functionally inactivated in approximately half of pancreatic carcinomas (e.g., Hahn et al., Science, 1996) and in lower proportions of other GI carcinomas (e.g., Moskaluk and Kern, Biochim, Biophys. Acta 1242:185, 1996, Hahn et al., Cancer Res. 58:1124, 1998). While 18q loss is seen in several other tumor types, homozygous loss of DPC4 is most commonly found in tumors of the GI tract (Schutte et al., Cancer Res. 56:2527, 1996). The protein product of DPC4 belongs to the evolutionary conserved family of SMAD proteins, which are involved in TGF-β signal transduction (see, e.g., Derynck and Feng, Biochim. Biophys. Acta. 1333:F105, 1997). DPC4/SMAD4 (SMA- and MAD-related protein 4) binds receptor-activated cytoplasmic SMADs and translocates them into the nucleus, where heteromeric nuclear SMAD4/SMADx complexes associate with DNA-binding proteins and activate gene transcription (Derynck and Feng, 1997).

RUNX3 belongs to a family of transcription factors involved in development and differentiation (Ito, Adv. Cancer Res. 99:33, 2008; Speck et al., Cancer Res. 59:1789s, 1999) and whose best characterized members are Runx1 and Runx2. The specificity of the upregulation in Runx3 in KP cells was underscored by the lack of significant changes in these close family members (−1.2-fold for Runx1 and 1.5-fold for Runx2; not shown). RUNX1 and RUNX2 have been identified as tumor suppressor genes (TSGs) in hematopoietic malignancies (Blyth et al., Nat. Rev. Cancer 5:376, 2005). Its alternate designation of AML2 notwithstanding, what little is known about the role of RUNX3 in malignancy comes primarily from studies of solid tumors, where it has been implicated on both sides of the cancer divide, and sometimes with contradicting conclusions in the same cancer.

Several studies point toward a tumor suppressive role for RUNX3 (e.g., Bae and Choi, Oncogene 23:4336, 2004), but a small number of reports indicate that it has oncogenic potential and that this potential might extend beyond murine lymphomas (Debernardi et al., Genes Chromosomes Cancer 37:149, 2003; Lacayo et al., Blood 104:2646, 2004). For instance, RUNX3 overexpression has been reported in basal cell (Salto-Tellez et al., Oncogene 25:7646, 2006), skin (Lee et al., Clin. Exp. Dermatol. 36:769, 2011b), head and neck (Tsunematsu et al., PLoS One 4:e5892, 2009) and ovarian cancers (Lee et al., Gynecol. Oncol. 122:410, 2011a). Finally, in gastric carcinoma, RUNX3 has been separately identified as either a TSG by some groups (e.g., Li et al., 2002) or an oncogene by others (e.g., Levanon et al., 2011). In addition, RUNX3 was identified as a possible downstream effector of TGFβ signaling by examining genes having upregulated expression in a DPC4 heterozygous mutant background (see PCT Application No. PCT/US2014/011536). Indeed, its specific role in tumorigenesis remains the subject of considerable controversy (see, also, Ito et al., Oncogene 28, 1379, 2009; Levanon et al., EMBO Mol. Med. 3:593, 2011; Levanon et al., Mech. Dev. 109:413, 2001; Li et al., Cell 109:113, 2002 and see below).

Prior to setting forth this disclosure in more detail, it may be helpful to an understanding thereof to provide definitions of certain terms to be used herein. Additional definitions are set forth throughout this disclosure.

In the present description, any concentration range, percentage range, ratio range, or integer range is to be understood to include the value of any integer within the recited range and, when appropriate, fractions thereof (such as one tenth and one hundredth of an integer), unless otherwise indicated. Also, any number range recited herein relating to any physical feature, such as polymer subunits, size or thickness, are to be understood to include any integer within the recited range, unless otherwise indicated. As used herein, the terms “about” and “consisting essentially of” mean±20% of the indicated range, value, or structure, unless otherwise indicated. It should be understood that the terms “a” and “an” as used herein refer to “one or more” of the enumerated components. The use of the alternative (e.g., “or”) should be understood to mean either one, both, or any combination thereof of the alternatives or enumerated components. As used herein, the terms “include,” “have” and “comprise” are used synonymously, which terms and variants thereof are intended to be construed as non-limiting.

The “percent identity” or “sequence identity,” as used herein, refers to the percentage of nucleic acid or amino acid residues in one sequence that are identical with the nucleic acid or amino acid residues in a reference polynucleotide or polypeptide sequence, respectively, (i.e., % identity=number of identical positions/total number of positions×100) after aligning the sequences and introducing gaps, if necessary, to achieve the maximum percent sequence identity of two or more sequences. For proteins, conservative substitutions are not considered as part of the sequence identity. The comparison of sequences and determination of percent identity between two or more sequences is accomplished using a mathematical algorithm, such as BLAST and Gapped BLAST programs at their default parameters (e.g., Altschul et al., J. Mol. Biol. 215:403, 1990; Altschul et al., Nucleic Acids Res. 25:3389, 1997; see also BLASTN or BLASTP at www.ncbi.nlm.nih.gov/BLAST).

A “conservative substitution” is recognized in the art as a substitution of one amino acid for another amino acid that has similar properties. Exemplary conservative substitutions are well known in the art (see, e.g., WO 97/09433, page 10, published Mar. 13, 1997; Lehninger, Biochemistry, Second Edition; Worth Publishers, Inc. NY:NY (1975), pp. 71-77; Lewin, Genes IV, Oxford University Press, NY and Cell Press, Cambridge, Mass. (1990), p. 8).

“Analog” as used herein refers to a compound that is structurally similar to a parent compound, but differs slightly in composition (e.g., one atom or functional group is different, added, or removed). An analog may or may not have different chemical or physical properties than the original compound and may or may not have improved biological or chemical activity. For example, an analog may be more hydrophilic or it may have altered activity as compared to a parent compound. An analog may mimic the chemical or biological activity of the parent compound (i.e., it may have similar or identical activity), or, in some cases, may have increased or decreased activity. In other cases, the changes in an analog may impart certain desirable properties (e.g., improved stability, improved bioavailability, improved hydrophilicity, minimized off-target effects, minimized toxicity). An analog may be a naturally or non-naturally occurring (e.g., chemically-modified or recombinant) variant of the original compound. An example of an analog is a mutein (i.e., a protein analog in which at least one amino acid is deleted, added, or substituted with another amino acid). An example of an RNA analog is an RNA molecule having a non-standard nucleotide, such as 5-methyuridine or 5-methylcytidine or 2-thioribothymidine. Other types of analogues include isomers (enantiomers, diastereomers, or the like) and other types of chiral variants of a compound, as well as structural isomers. An analog may be a branched or cyclic variant of a linear compound.

As used herein, the term “derivative” refers to a modification of a compound by chemical or biological means, with or without an enzyme, which modified compound is structurally similar to a parent compound and (actually or theoretically) derivable from that parent compound. Generally, a “derivative” differs from an “analog” in that a parent compound may be the starting material to generate a “derivative,” whereas the parent compound may not necessarily be used as the starting material to generate an “analog.” A derivative may have different chemical, biological or physical properties of the parent compound, such as being more hydrophilic or having altered reactivity as compared to the parent compound. Derivatization (i.e., modification) may involve substitution of one or more moieties within the molecule (e.g., a change in functional group). For example, a hydrogen may be substituted with a halogen, such as fluorine or chlorine, or a hydroxyl group (—OH) may be replaced with a carboxylic acid moiety (—COOH). Other exemplary derivatizations include glycosylation, alkylation, acylation, acetylation, ubiqutination, esterification, and amidation.

The term “derivative” also refers to all solvates, for example hydrates or adducts (e.g., adducts with alcohols), active metabolites, and salts of the parent compound. The type of salt that may be prepared depends on the nature of the moieties within the compound. For example, acidic groups such as carboxylic acid groups can form alkali metal salts or alkaline earth metal salts (e.g., sodium salts, potassium salts, magnesium salts and calcium salts, and also salts with physiologically tolerable quaternary ammonium ions and acid addition salts with ammonia and physiologically tolerable organic amines such as, for example, triethylamine, ethanolamine or tris-(2-hydroxyethyl)amine). Basic groups can form acid addition salts, for example, with inorganic acids such as hydrochloric acid, sulfuric acid or phosphoric acid, or with organic carboxylic acids and sulfonic acids such as acetic acid, citric acid, lactic acid, benzoic acid, maleic acid, fumaric acid, tartaric acid, methanesulfonic acid or p-toluenesulfonic acid. Compounds that simultaneously contain a basic group and an acidic group, for example, a carboxyl group in addition to basic nitrogen atoms, can be present as zwitterions. Salts can be obtained by customary methods known to those skilled in the art, for example, by combining a compound with an inorganic or organic acid or base in a solvent or diluent, or from other salts by cation exchange or anion exchange.

Other types of derivatives include conjugates and prodrugs of a parent compound (i.e., chemically modified derivatives which can be converted into the original compound under physiological conditions). For example, the prodrug may be an inactive form of an active agent. Under physiological conditions, the prodrug may be converted into the active form of the compound. Prodrugs may be formed, for example, by replacing one or two hydrogen atoms on nitrogen atoms by an acyl group (acyl prodrugs) or a carbamate group (carbamate prodrugs). More detailed information relating to prodrugs may be found in, for example, Fleisher et al., Adv. Drug Del. Rev 19:115, 1996; Design of Prodrugs, H. Bundgaard (ed.), Elsevier, 1985; or Bundgaard, Drugs of the Future 16:443, 1991.

As used herein, the term “isolated” means that the molecule referred to is removed from its original environment, such as being separated from some or all of the co-existing materials in a natural environment (e.g., a natural environment may be a cell).

The term “biological sample” includes a blood sample, biopsy specimen, tissue explant, organ culture, biological fluid (e.g., serum, urine, CSF) or any other tissue or cell or other preparation from a subject or a biological source. A subject or biological source may, for example, be a human or non-human animal, a primary cell culture or culture adapted cell line including genetically engineered cell lines that may contain chromosomally integrated or episomal recombinant nucleic acid molecules, somatic cell hybrid cell lines, immortalized or immortalizable cell lines, differentiated or differentiatable cell lines, transformed cell lines, or the like.

In further embodiments of this disclosure, a subject or biological source may be suspected of having or being at risk for having a disease, disorder or condition, including a malignant disease, disorder or condition (e.g., pancreatic cancer). In certain embodiments, a subject or biological source may be suspected of having or being at risk for having a hyperproliferative disease (e.g., pancreatic cancer), and in certain other embodiments of this disclosure the subject or biological source may be known to be free of a risk or presence of such disease, disorder, or condition.

By “subject” is meant an organism having, or at risk of having or recurring, pancreas cancer cells. A subject may benefit from a particular therapeutic regimen described herein, which can be based on, for example, a DPC4 genotype in combination with a RUNX3 expression level of pancreas cancer cells, or based on a RUNX3 expression level and one or more of a Col6a1 expression level and an Spp1 expression level in the pancreas cancer cells. “Subject” also refers to an organism to which a small molecule, chemical entity, nucleic acid molecule, peptide or polypeptide of this disclosure can be administered to treat, ameliorate or prevent recurrence of pancreas cancer. In certain embodiments, a subject is an animal, such as a mammal or a primate. In other embodiments, a subject is a human or a non-human primate.

“Treatment,” “treating” or “ameliorating” refers to either a therapeutic treatment or prophylactic/preventative treatment. A treatment is therapeutic if at least one symptom of disease (e.g., pancreatic cancer) in an individual receiving treatment improves or a treatment may delay worsening of a progressive disease in an individual, or prevent onset of additional associated diseases (e.g., metastases from pancreatic cancer).

A “therapeutically effective amount (or dose)” or “effective amount (or dose)” refers to that amount of compound sufficient to result in amelioration of one or more symptoms of the disease being treated (e.g., pancreatic cancer) in a statistically significant manner. When referring to an individual active ingredient, administered alone, a therapeutically effective dose refers to that ingredient alone. When referring to a combination, a therapeutically effective dose refers to combined amounts of the active ingredients that result in the therapeutic effect, whether administered serially or simultaneously (in the same formulation or in separate formulations).

The term “pharmaceutically acceptable” refers to molecular entities and compositions that do not produce allergic or other serious adverse reactions when administered using routes well known in the art.

A “patient in need” or “subject in need” refers to a patient or subject at risk of, or suffering from, a disease, disorder or condition (e.g., pancreatic cancer) that is amenable to treatment or amelioration with a therapy regimen as provided herein.

As used herein, the term “expression level” refers to the quantity of protein and/or gene expression by a cell or population of cells. Techniques for detecting and measuring protein expression are known to those of skill in the art and include, for example, immunostaining, immunoprecipitation, fluorescence-labeling, BCA, and Western blot. Techniques for detecting and measuring gene expression are known to those of skill in the art and include, for example, RT-PCR, in situ hybridization, fluorescence-labeled oligonucleotide probes, radioactively labeled oligonucleotide probes, and Northern blot.

As used herein, “genotype” means the genetic makeup of a cell or an organism with reference to a particular trait or set of traits. For example, a cell having zero copies of a nucleic acid molecule that encodes DPC4 has a “homozygous null genotype” for DPC4. In another example, a cell having one copy of a nucleic acid molecule that encodes DPC4 and one copy of a nucleic acid molecule that does not encode or express DPC4 has a “heterozygous genotype” for DPC4. In another example, a cell having two or more copies of a nucleic acid molecule that encodes DPC4 and no nucleic acid molecule that do not encode or express DPC4 has a “homozygous positive genotype” for DPC5.

Techniques for determining genotype for a given trait, such as encoding or expressing a gene, are known to those of skill in the art and include, for example, polymerase chain reaction (PCR) followed by one or more of gel electrophoresis or DNA sequencing. Alternatively, genotype may be determined by performing a restriction enzyme digest of sufficient amount of DNA and subsequently analyzing the products of the digest.

The phrase “a single copy,” as used herein, refers to a condition in which one or more copies of DNA corresponding to a gene are lost and only one copy remains. For example, in a cell that with two copies of DNA corresponding to the DPC4 gene and a heterozygous genotype for DPC4, loss of one copy of the DNA results in the cell having only a single copy, which is either positive for DPC4 or null for DPC4.

“Metastatic spread,” as used herein, refers to a process by which cancer cells from a first organ or part of the body move to and proliferate in a second organ or part of the body that is not directly connected with the first.

“Metastatic potential,” as used herein, refers to the capacity of cancer cells for metastatic spread, and includes the genotype of the cancer cells for a gene or genes associated with metastatic spread, the expression levels of the gene or genes, or the expression levels of a protein or proteins that are associated with metastatic spread. Other factors may influence metastatic potential, such as the location of the cancer cells within the subject, or within a given tissue, organ, or other body part. When the subject is a human, the age, gender, overall health, diet, past health history, and past or current cancer therapies administered to the subject may also influence metastatic potential.

“Tumor growth potential,” as used herein, refers to the capacity of cancer cells in a tumor to proliferate within a tumorous tissue and, in some instances, to adjacent tissue in the same or a different organ or part of the body. While metastatic potential reflects the capacity for cancer cells to move to and proliferate in a distant organ or part of the body, tumor growth potential reflects the capacity for cancer cells to proliferate, in some instances, move within a local setting.

The phrase “local recurrence” refers to the renewed appearance or proliferation of cancer cells at the same or an adjacent site (e.g., the same or an adjacent location within a tissue, organ, or body part) following a therapeutic regimen to remove or treat the cancer cells.

As used herein, the term “nanobody” refers to an antibody fragment consisting of a single monomeric variable domain of a heavy-chain antibody. Nanobodies bind selectively to a specific antigen and, being smaller in size relative to antibodies, may bind smaller targets and may be favored over antibodies for cell transformation.

As used herein, the terms “antibody” or “binding fragment”, or “antibody fragment” refer to their standard meanings within the art; that is, an intact immunoglobulin molecule or a fragment thereof that is capable of binding an antigen.

The term “T cell receptor”, as used herein, refers to an heterodimeric antigen binding receptor derived from a T lymphocyte, comprising a an alpha/beta polypeptide dimer or a gamma/delta polypeptide dimer, each dimer comprising a variable region, a constant region, and an antigen binding site.

The phrase “main therapy” refers to a surgical intervention to treat a pancreas cancer or pancreas cancer precursor lesion.

“Neoadjuvant therapy,” as used herein, refers to a therapeutic intervention prior to or contemporaneous with a main therapy, such as a surgical procedure. For example, a neoadjuvant therapy may be administered to a subject presenting with pancreas cancer or a pancreas cancer precursor lesion prior to or contemporaneous with the subject undergoing a resection procedure that removes all or part of a cancerous tissue or lesion.

“Adjuvant therapy,” as used herein, refers to a therapy administered to a subject following a main therapy such as a surgical procedure. For example, after a subject has undergone a resection procedure to remove some or all of a cancerous or pre-cancerous tissue from the subject's pancreas, an adjuvant therapy (e.g., chemotherapy, radiotherapy) may be administered.

“Systemic therapy,” as used herein, refers to a therapy that is not targeted to a particular organ or other body part, but is instead acts globally (even if administered locally), without specific regard to whether a given organ or other body part receiving the therapy has or is at risk for developing a cancer. In certain embodiments, a neoadjuvant therapy, an adjuvant therapy, or both, includes a systemic therapy, such as systemic administration of chemotherapy, combined chemotherapies, biologic therapy, hormonal therapy, or any combination thereof.

“Localized therapy,” as used herein, refers to a therapy that is targeted to those organs or other parts of the body that have or are at risk for developing a cancer. In certain embodiments, a neoadjuvant therapy, an adjuvant therapy, or both, includes a localized therapy, such as localized administration of radiotherapy, chemotherapy, combined chemotherapies, biologic therapy, hormonal therapy, or any combination thereof.

As used herein, the term “immunoreactive T cell” refers to a naturally occurring or engineered cytotoxic T lymphocyte (i.e., a CD8+ T cell) capable of killing a damaged or infected cell, and/or to a naturally occurring or engineered T helper cell (i.e., a CD4+ T cell) capable of effecting an immune response within the subject when presented with an antigen by an MHC1 marker.

As used herein, the term “immunoreactive Natural Killer cell” refers to a naturally occurring or engineered cytotoxic lymphocyte of the innate immune system which is distinct from a cytotoxic T lymphocyte and which is capable of recognized and killing a damaged or infected cell without prior activation by MHC1 markers.

### Diagnosing or Detecting Metastatic Potential of Pancreas Cancer Cells

The cellular demands of proliferation and dissemination appear to compete in pancreas cancers, at least until DPC4 signaling is completely lost. The instant disclosure shows that partial attenuation of TGFβ signaling via DPC4 shifts the burden of disease toward primary tumor growth by lowering barriers to proliferation at the expense of sacrificing mediators of metastasis. Governing this switch is the transcription factor Runx3, which integrates inputs from DPC4 and TRP53 (FIG. 6G) to generate three broad sets of biological behavior. These shifts in pathobiology with mutational state are consistent with patterns of spread observed in patients. At one extreme are patients with locally invasive and destructive tumors and minimal discernible dissemination; at the other are patients with a more restricted primary tumor but widespread metastases. Complete loss of DPC4 in the context of point mutant Trp53 reveals a third state: uninhibited primary tumor growth together with high metastatic potential.

It may seem paradoxical that diminution of DPC4 signaling can impede dissemination, given that loss of expression frequently, though not always (Iacobuzio-Donahue et al., J. Clin. Oncol. 27:1806, 2009), occurs in PDA metastases. As disclosed herein, signaling levels impact dissemination. Approximately half of conventional PDA lose expression of DPC4/SMAD4 and this is thought to represent a late event (Wilentz et al., Cancer Res. 60:2002, 2000). As these data are derived from IHC observations, they are generally bimodal (i.e., all or none) and cannot reliably report heterozygous loss of DPC4. In autopsy series of PDA patients, a range of metastatic disease burdens has been observed, with death not infrequently attributable to local destruction as opposed to disseminated disease (Iacobuzio-Donahue et al., 2009). Two distinct patterns of metastatic spread emerge: widely metastatic disease characteristic of the majority of cases and an oligometastatic state in a minority. In light of this disclosure, the most aggressive disease presentation plausibly involves heterozygous mutation of DPC4 relatively early in disease progression, promoting aggressive local growth and invasion; subsequent loss of DPC4 expression from the remaining allele in a subclone would then give rise to a highly metastatic subpopulation. Indeed, in the few KPDC animals that showed metastatic disease, the tumor cells in those metastases had frequently lost DPC4 expression. In this setting, survival would be threatened by the sequelae of both local and disseminated disease burdens in a highly lethal combination.

In one aspect, the present disclosure provides methods for diagnosing or detecting metastatic potential of pancreas cancer cells. In certain embodiments, methods for diagnosing or detecting metastatic potential of pancreas cancer cell comprise determining whether pancreas cancer cells from a mammalian subject comprise (i) a homozygous positive genotype for DPC4 or have a single copy of DPC4, wherein the single copy is positive for DPC4, and have a RUNX3 expression level that is increased compared to normal cells from the subject, the pancreas cancer cells thereby having an increased metastatic potential as compared to tumor growth potential; or (ii) a heterozygous genotype for DPC4 and have a RUNX3 expression at a level similar to normal cells from the subject, the pancreas cancer cells thereby not having an increased metastatic potential as compared to tumor growth potential; or (iii) homozygous null genotype for DPC4 or have a single copy of DPC4, wherein the single copy is null for DPC4, and have a RUNX3 expression level that is increased as compared to normal cells from the subject, the pancreas cancer cells thereby having metastatic potential and/or tumor growth potential.

In certain other embodiments, methods for diagnosing or detecting metastatic potential of cancer cells comprise determining whether the pancreas cancer cells from a mammalian subject have: (i) a RUNX3 expression level that is increased as compared to normal cells from the subject, and an expression level of Spp1, Col6a1, or both, that is increased as compared to normal cells from the subject, the pancreas cancer cells thereby having an increased metastatic potential as compared to tumor growth potential; or (ii) a RUNX3 expression level that is increased as compared to normal cells from the subject, and an expression level of Spp1, Col6a1, or both, that is decreased or similar as compared to normal cells from the subject, the pancreas cancer cells thereby not having an increased metastatic potential as compared to tumor growth potential.

In yet another aspect, kits are provided for diagnosing or detecting, in pancreas cancer cells from a mammalian subject, a metastatic potential, a tumor growth potential, or both, wherein a kit comprises an oligonucleotide primer set specific for DPC4, a binding agent specific for RUNX3 and wherein an increased metastatic potential as compared to tumor growth potential is present when pancreas cancer cells from a mammalian subject comprise a homozygous positive genotype for DPC4 or have a single copy of DPC4, wherein the single copy is positive for DPC4, and have a RUNX3 expression level that is increased as compared to normal cells from the subject; or wherein an increased tumor growth potential as compared to metastatic potential is present when pancreas cancer cells from the mammalian subject comprise a heterozygous genotype for DPC4 and have a RUNX3 expression level that is similar to normal cells from the subject; or wherein a metastatic potential and/or a tumor growth potential is present when pancreas cancer cells from the mammalian subject comprise a homozygous null genotype for DPC4 or have a single copy of DPC4, wherein the single copy is null for DPC4, and have a RUNX3 expression level that is increased as compared to normal cells from the subject.

In certain embodiments, the binding agent specific for RUNX3 comprises a nanobody or a binding fragment thereof, an antibody or a binding fragment thereof, or a T-cell receptor molecule or a binding fragment thereof.

In certain further embodiments, the binding agent is conjugated to a detectable agent. In certain further embodiments, the binding agent is detectable by one or more of: a colorimetric assay, fluorescence imaging, an enzymatic assay, spectrophotometry, mass spectroscopy, or radiation imaging.

In certain embodiments, a kit further comprises one or more of: instructions for using the primer and the detectable agent; reagents for performing a PCR reaction; and reagents for performing a binding reaction using the detectable agent.

### Methods of Treating or Reducing Risk of Pancreas Cancer Progression or Recurrence

In other aspects, the present disclosure provides methods for reducing a risk of pancreas cancer progression or recurrence, and methods for treating pancreas cancer, which methods involve identifying an appropriate therapeutic regimen based on the genotype and expression levels of the pancreas cancer cells, as disclosed herein. Therapeutic regimens disclosed herein comprise a main therapy, such as a surgical procedure, in combination with one or more of a neoadjuvant therapy or an adjuvant therapy.

For pancreas cancer or a pancreas cancer precursor lesion, several therapeutic regimens are known in the art. For example, the Whipple procedure, or pancreaticoduodenectomy, is the most commonly performed surgery to remove pancreatic tumors. Pancreatic cancer is considered resectable if the tumor appears to be localized to the pancreas without invasion into important surrounding structures, such as the mesenteric blood vessels (that supply blood to the intestines) located adjacent to the head portion of the pancreas. Furthermore there should be no evidence of metastatic spread to the liver or to the peritoneum. In a standard Whipple operation, a surgeon will remove the head of the pancreas, the gallbladder, part of the duodenum (i.e., the uppermost portion of the small intestine), a small portion of the stomach called the pylorus, and the lymph nodes near the head of the pancreas. Then the remaining pancreas and digestive organs are reconnected so that pancreatic digestive enzymes, bile, and stomach contents will flow into the small intestine during digestion. In another type of Whipple procedure, known as pylorus preserving Whipple, the bottom portion of the stomach, or pylorus, is not removed. In either case, such a surgery can last from about 6 hours to about 10 hours.

When pancreatic cancer has grown beyond the confines of the pancreas to invade surrounding vital structures, such a locally advanced pancreatic cancer is not treated by surgery. Treatment of locally advanced pancreatic cancer includes one or more of a neoadjuvant therapy and an adjuvant therapy. In certain embodiments, one or more of the neoadjuvant therapy and an adjuvant therapy comprises chemotherapy, radiation therapy, or both. Exemplary chemotherapeutics used for the treatment of pancreatic cancer include 5-fluorouracil, leucovorin, gemcitabine, cisplatin, irinotecan, paclitaxel, nanoparticle albumin bound (nab)-paclitaxel, docetaxel, capecitabine, oxaliplatin, and the FOLFIRINOX combination (5-fluorouracil, leucovorin, irinotecan and oxaliplatin). Exemplary radiation therapy is delivered in daily fractions over a six week period to a total dose of approximately 5,000 rads, which may be external (e.g., high energy X-rays) or internal (e.g., radiation contained in needles, seeds, wires, or catheters, which are placed directly into or near a tumor). In certain embodiments, chemotherapy may be administered together or sequentially with the radiation therapy.

Exemplary chemotherapeutic agents include alkylating agents (e.g., cisplatin, oxaliplatin, carboplatin, busulfan, nitrosoureas, nitrogen mustards such as bendamustine, uramustine, temozolomide), antimetabolites (e.g., aminopterin, methotrexate, mercaptopurine, fluorouracil, cytarabine, gemcitabine), taxanes (e.g., paclitaxel, nab-paclitaxel, docetaxel), anthracyclines (e.g., doxorubicin, daunorubicin, epirubicin, idaruicin, mitoxantrone, valrubicin), bleomycin, mytomycin, actinomycin, hydroxyurea, topoisomerase inhibitors (e.g., camptothecin, topotecan, irinotecan, etoposide, teniposide), monoclonal antibodies (e.g., ipilimumab, pembrolizumab, nivolumab, avelumab, alemtuzumab, bevacizumab, cetuximab, gemtuzumab, panitumumab, rituximab, tositumomab, trastuzumab), vinca alkaloids (e.g., vincristine, vinblastine, vindesine, vinorelbine), cyclophosphamide, prednisone, leucovorin, oxaliplatin, hyalurodinases, or any combination thereof.

Chemotherapy or radiation therapy may be likewise administered separately, or, alternatively, together to treat pancreas cancer undergoing metastatic spread or that has increased metastatic potential. In certain embodiments, a chemotherapy and a radiation therapy are administered contemporaneously. In certain other embodiments, a chemotherapy and a radiation therapy are administered sequentially in any order.

In certain embodiments, a neoadjuvant therapy or an adjuvant therapy further or alternatively comprise one or more of combined chemotherapies, biologic therapy, hormonal therapy, or any combination thereof.

In certain embodiments, a biologic therapy includes an antibody, an scFv, a nanobody, a fusion protein (e.g., chimeric antigen receptor (CAR), such as used in adoptive immune therapy comprising a T cell expressing an antigen specific CAR), a tyrosine kinase inhibitor, an immunoreactive T cell, an immunoreactive Natural Killer cell, or any combination thereof. In certain further embodiments, an antibody comprises ipilimumab, pembrolizumab, nivolumab, avelumab, cetuximab, trastuzumab, bevacizumab, alemtuzumab, gemtuzumab, panitumumab, rituximab, tositumomab, anti-CD44 antibody, anti-Spp1 antibody, or any combination thereof.

In certain embodiments, a neoadjuvant therapy, an adjuvant therapy, or both comprise or further comprise administering to the subject an expression or activity inhibitor of RUNX3, Bmpr1a, Smad5, Tgfb3, Smad4, Bmp1, Itgb7, Tgfb1i1, Tgfb1, Bmper, Ltbp1, Ltbp2, Itgb5, Id1, Tgfbi, Dlx2, or any combination thereof. Still further exemplary therapies for use in the therapy regimens of this disclosure include expression or activity inhibitors of cyclin D, cyclin E, Ctgf, Selp, Timp2, Col5a1, Ncam1, Thbs3, Mmp11, Sgce, Fn1, Vcam1, Ecm1, Adamts1, Mmp2, Thbs1, Fbln1, Tgfbi, Cdh2, Mmp10, Timp3, Spp1, Vcan, Sparc, Col6a1, a CD44 antagonist such as an anti-CD44 antibody, an Spp1 antagonist such as an anti-SPP1 antibody, or any combination thereof.

To practice coordinate administration methods of this disclosure, therapy regimens combine main therapies (e.g., a tumor resection procedure) with additional therapies (e.g., one or more of a neoadjuvant therapy or an adjuvant therapy) simultaneously or sequentially in a coordinated treatment protocol. For example, a therapy regimen may combine a tumor resection procedure with an optional neoadjuvant therapy comprising chemotherapy and an adjuvant therapy comprising radiation therapy and chemotherapy. In this example, an optional neoadjuvant therapy may comprise one or more chemotherapeutic agents to be administered concurrently or sequentially, in a given order or otherwise. Similarly, an adjuvant therapy in this example may comprise coordinate or sequential administration of chemotherapy and radiation therapy, wherein the chemotherapy may comprise coordinate or sequential administration of one or more chemotherapeutic agent.

A coordinate administration of one or more therapies or agents may be done in any order, and there may be a time period while only one or both (or all) therapies, individually or collectively, exert their biological activities. A distinguishing aspect of all such coordinate treatment methods is that a treatment regimen elicits some favorable clinical response, which may or may not be in conjunction with a secondary clinical response provided by an additional therapeutic agent or process. For example, the coordinate administration of main and adjuvant or neoadjuvant therapies as contemplated herein can yield an enhanced (e.g., synergistic) therapeutic response beyond the therapeutic response elicited by any of the therapies alone.

In further embodiments, provided are compositions for reducing the risk of or treating pancreatic cancer (e.g., tumor) or metastatic pancreatic cancer comprising an inhibitor of RUNX3 expression or activity (such as small molecules, chemical entities, nucleic acid molecules, peptides or polypeptides) and a pharmaceutically acceptable carrier, diluent or excipient. In certain embodiments, a composition contains a RUNX3 inhibitor that is a dsRNA (interfering RNA) or antisense RNA. In certain further embodiments, a RUNX3 inhibitor comprises an agent that increases the degradation of a RUNX3 transcript or protein. Exemplary RUNX3 inhibitors are disclosed in, for example, PCT Publication No. WO 2014/113406, which RUNX3 inhibitors are incorporated herein by reference in their entirety.

The term “dsRNA” as used herein refers to any nucleic acid molecule comprising at least one ribonucleotide and is capable of inhibiting or down regulating gene expression, for example, by promoting RNA interference (“RNAi”) or gene silencing in a sequence-specific manner. The dsRNAs of the instant disclosure may be suitable substrates for Dicer or for association with RISC to mediate gene silencing by RNAi. One or both strands of the dsRNA can further comprise a terminal phosphate group, such as a 5′-phosphate or 5′,3′-diphosphate. As used herein, dsRNA molecules, in addition to at least one ribonucleotide, can further include substitutions, chemically-modified nucleotides, or non-nucleotides. In certain embodiments, dsRNA molecules comprise ribonucleotides at up to about 100% of the nucleotide positions.

In addition, as used herein, the term dsRNA is meant to be equivalent to other terms used to describe nucleic acid molecules that are capable of mediating sequence specific RNAi, for example, nicked dsRNA (ndsRNA), gapped dsRNA (gdsRNA), short interfering nucleic acid (siNA), siRNA, micro-RNA (miRNA), short hairpin RNA (shRNA), short interfering oligonucleotide, short interfering substituted oligonucleotide, short interfering modified oligonucleotide, chemically-modified dsRNA, post-transcriptional gene silencing RNA (ptgsRNA), or the like. The term “large double-stranded (ds) RNA” refers to any double-stranded RNA longer than about 40 base pairs (bp) to about 100 bp or more, particularly up to about 300 bp to about 500 bp. The sequence of a large dsRNA may represent a segment of an mRNA or an entire mRNA. A double-stranded structure may be formed by self-complementary nucleic acid molecule or by annealing of two or more distinct complementary nucleic acid molecule strands.

In one aspect, a dsRNA comprises two separate oligonucleotides, comprising a first strand (antisense) and a second strand (sense), wherein the antisense and sense strands are self-complementary (i.e., each strand comprises a nucleotide sequence that is complementary to a nucleotide sequence in the other strand and the two separate strands form a duplex or double-stranded structure, for example, wherein the double-stranded region is about 15 to about 24 or 25 base pairs or about 25 or 26 to about 40 base pairs); the antisense strand comprises a nucleotide sequence that is complementary to a nucleotide sequence of a target nucleic acid molecule or a portion thereof (e.g., RUNX3); and the sense strand comprises a nucleotide sequence corresponding (i.e., homologous) to the target nucleic acid sequence or a portion thereof (e.g., a sense strand of about 15 to about 25 nucleotides or about 26 to about 40 nucleotides corresponds to the target nucleic acid or a portion thereof).

A dsRNA or large dsRNA may include a substitution or modification in which the substitution or modification may be in a phosphate backbone bond, a sugar, a base, or a nucleoside. Such nucleoside substitutions can include natural non-standard nucleosides (e.g., 5-methyluridine or 5-methylcytidine or a 2-thioribothymidine), and such backbone, sugar, or nucleoside modifications can include an alkyl or heteroatom substitution or addition, such as a methyl, alkoxyalkyl, halogen, nitrogen or sulfur, or other modifications known in the art.

As used herein, the term “RNAi” is meant to be equivalent to other terms used to describe sequence specific RNA interference, such as post transcriptional gene silencing, translational inhibition, or epigenetics. For example, dsRNA and single stranded antisense molecules of this disclosure can be used to epigenetically silence genes at the post-transcriptional level, the pre-transcriptional level, or any combination thereof.

As used herein, “gene silencing” refers to a partial or complete loss-of-function through targeted inhibition of gene expression in a cell, which may also be referred to as RNAi “knockdown,” “inhibition,” “down-regulation,” or “reduction” of expression of a target gene. Depending on the circumstances and the biological problem to be addressed, it may be preferable to partially reduce gene expression. Alternatively, it might be desirable to reduce gene expression as much as possible. The extent of silencing may be determined by methods described herein and known in the art, some of which are summarized in PCT Publication No. WO 99/32619. Depending on the assay, quantification of gene expression permits detection of various amounts of inhibition that may be desired in certain embodiments of this disclosure, including prophylactic and therapeutic methods, which will be capable of knocking down target gene expression, in terms of mRNA level or protein level or activity, for example, by equal to or greater than 10%, 30%, 50%, 75% 90%, 95% or 99% of baseline (i.e., normal) or other control levels, including elevated expression levels as may be associated with particular disease states or other conditions targeted for therapy.

For the purposes of administration, the compounds of the present disclosure may be administered as a raw chemical or may be formulated as pharmaceutical compositions. Pharmaceutical compositions of the present disclosure may comprise a small molecule, chemical entity, nucleic acid molecule, peptide or polypeptide, and a pharmaceutically acceptable carrier, diluent or excipient. The small molecule, chemical entity, nucleic acid molecule, peptide or polypeptide composition will be in an amount that is effective to treat a particular disease or condition of interest—that is, in an amount sufficient for reducing the risk of or treating pancreatic cancer, metastases arising from the pancreatic cancer, a pancreatic cancer precursor lesion, a metastatic niche associated with pancreatic cancer or any of the other associated indication described herein, and preferably with acceptable toxicity to a patient. Compounds for use in the methods described herein can be determined by one skilled in the art, for example, as described in the Examples below. Appropriate concentrations and dosages can be readily determined by one skilled in the art.

Administration of the compounds of this disclosure, or their pharmaceutically acceptable salts, in pure form or in an appropriate pharmaceutical composition, can be carried out using any mode of administration for agents serving similar utilities. The pharmaceutical compositions of this disclosure can be prepared by combining a compound of this disclosure with an appropriate pharmaceutically acceptable carrier, diluent or excipient, and may be formulated into preparations in solid, semi-solid, liquid or gaseous forms, such as tablets, capsules, powders, granules, ointments, solutions, suppositories, injections, inhalants, gels, microspheres, and aerosols. Exemplary routes of administering such pharmaceutical compositions include oral, topical, transdermal, inhalation, parenteral, sublingual, buccal, rectal, vaginal, and intranasal.

The term “parenteral” as used herein includes subcutaneous injections, intravenous, intramuscular, intrasternal injection or infusion techniques. Pharmaceutical compositions of this disclosure are formulated to allow the active ingredients contained therein to be bioavailable upon administration of the composition to a patient. Compositions that will be administered to a subject or patient take the form of one or more dosage units, where for example, a tablet may be a single dosage unit, and a container of a compound of this disclosure in aerosol form may hold a plurality of dosage units. Actual methods of preparing such dosage forms are known, or will be apparent, to those skilled in this art (see, e.g., Remington: The Science and Practice of Pharmacy, 22^{nd} Edition (Pharmaceutical Press, 2012). The composition to be administered will, in any event, contain a therapeutically effective amount of a compound of this disclosure, or a pharmaceutically acceptable salt thereof, for reducing the risk of or treating pancreatic cancer, metastases arising from the pancreatic cancer, a pancreatic cancer precursor lesion, a metastatic niche associated with pancreatic cancer or other condition of interest in accordance with the teachings of this disclosure.

As a solid composition for oral administration, the pharmaceutical composition may be formulated into a powder, granule, compressed tablet, pill, capsule, chewing gum, wafer or the like form. Exemplary solid compositions can contain one or more inert diluents or edible carriers. In addition, one or more additives may be present, including binders such as carboxymethylcellulose, ethyl cellulose, microcrystalline cellulose, gum tragacanth or gelatin; excipients such as starch, lactose or dextrins, disintegrating agents such as alginic acid, sodium alginate, Primogel, corn starch and the like; lubricants such as magnesium stearate or Sterotex; glidants such as colloidal silicon dioxide; sweetening agents such as sucrose or saccharin; a flavoring agent such as peppermint, methyl salicylate or orange flavoring; or a coloring agent. When a pharmaceutical composition is in the form of a capsule, such as a gelatin capsule, it may contain, in addition to materials of the above type, a liquid carrier such as polyethylene glycol or oil or combinations thereof.

The pharmaceutical composition may be in the form of a liquid, such as an elixir, syrup, solution, emulsion, or suspension. In certain embodiments, a liquid composition may be formulated for oral administration or for delivery by injection, as two examples. When intended for oral administration, exemplary compositions may further contain, in addition to one or more compounds of this disclosure, a sweetening agent, preservative, dye/colorant, flavor enhancer, or any combination thereof. Exemplary compositions intended for administration by injection may further contain a surfactant, preservative, wetting agent, dispersing agent, suspending agent, buffer, stabilizer, isotonic agent, or any combination thereof.

Liquid pharmaceutical compositions of this disclosure, whether they are solutions, suspensions or other like forms, may further comprise adjuvants, including sterile diluents such as water for injection, saline solution, preferably physiological saline, Ringer's solution, isotonic sodium chloride, fixed oils such as synthetic mono or diglycerides which may serve as the solvent or suspending medium, polyethylene glycols, glycerin, propylene glycol or other solvents; antibacterial agents such as benzyl alcohol or methyl paraben; antioxidants such as ascorbic acid or sodium bisulfate; chelating agents such as ethylenediaminetetraacetic acid (EDTA); buffers such as acetates, citrates or phosphates and agents for the adjustment of tonicity such as sodium chloride or dextrose. The parenteral preparation can be enclosed in ampoules, disposable syringes or multiple dose vials made of glass or plastic. Physiological saline is a preferred adjuvant. An injectable pharmaceutical composition is preferably sterile.

A pharmaceutical composition of this disclosure may be intended for topical administration, in which case the carrier may comprise a suitable solution, emulsion, ointment, gel base, or any combination thereof. The base, for example, may comprise petrolatum, lanolin, polyethylene glycols, bee wax, mineral oil, diluents such as water and alcohol, emulsifiers, stabilizers, or any combination thereof. Thickening agents may be present in a pharmaceutical composition of this disclosure for topical administration. If intended for transdermal administration, the composition may include a transdermal patch or iontophoresis device.

A pharmaceutical composition of this disclosure may be intended for rectal administration, in the form, for example, of a suppository, which will melt in the rectum and release the active compound(s). A composition for rectal administration may contain an oleaginous base as a suitable nonirritating excipient. Exemplary bases include lanolin, cocoa butter, polyethylene glycol, or any combination thereof.

A pharmaceutical composition of this disclosure may include various materials that modify the physical form of a solid or liquid dosage unit. For example, a composition may include materials that form a coating shell around the active ingredient(s). Exemplary materials for forming a coating shell may be inert, such as sugar, shellac, or other enteric coating agents. Alternatively, active ingredient(s) may be encased in a gelatin capsule.

In certain embodiments, compounds and compositions of this disclosure may be in the form of a solid or liquid. Exemplary solid or liquid formulations include semi-solid, semi-liquid, suspension, and gel forms. A pharmaceutical composition of this disclosure in solid or liquid form may further include an agent that binds to the compound of this disclosure and thereby assists in the delivery of the compound. Suitable agents that may act in this capacity include a monoclonal or polyclonal antibody, a protein, or a liposome.

A pharmaceutical composition of this disclosure may consist of dosage units that can be administered as an aerosol. The term aerosol is used to denote a variety of systems ranging from those of colloidal nature to systems consisting of pressurized packages. Delivery may be by a liquefied or compressed gas or by a suitable pump system that dispenses the active ingredients. Aerosols of compounds of this disclosure may be delivered in single phase, bi-phasic, or tri-phasic systems in order to deliver the active ingredient(s). Delivery of the aerosol includes the necessary container, activators, valves, subcontainers, and the like, which together may form a kit.

Pharmaceutical compositions of this disclosure may be prepared by methodology well known in the pharmaceutical art. For example, a pharmaceutical composition intended to be administered by injection can be prepared by combining a compound of this disclosure with sterile, distilled water to form a solution. A surfactant may be added to facilitate the formation of a homogeneous solution or suspension. Surfactants are compounds that non-covalently interact with the compound of this disclosure to facilitate dissolution or homogeneous suspension of a compound in an aqueous delivery system.

Compounds of this disclosure, or their pharmaceutically acceptable salts, are administered in a therapeutically effective amount, which will vary depending upon a variety of factors including the activity of the specific compound employed; the metabolic stability and length of action of the compound; the age, body weight, general health, sex, and diet of the patient; the mode and time of administration; the rate of excretion; the drug combination; the severity of the particular disorder or condition; and the subject undergoing therapy. Following administration of therapies according to the formulations and methods of this disclosure, test subjects will exhibit about a 10% up to about a 99% reduction in one or more symptoms associated with the disease or disorder being treated (e.g., pancreas cancer), as compared to placebo-treated or other suitable control subjects.

Compounds of this disclosure, or pharmaceutically acceptable derivatives thereof, may also be administered simultaneously with, prior to, or after administration of one or more other therapeutic agents. Such combination therapy includes administration of a single pharmaceutical dosage formulation which contains a compound of this disclosure and one or more additional active agents, as well as administration of the compound of this disclosure and each active agent in its own separate pharmaceutical dosage formulation. For example, a compound of this disclosure and the other active agent can be administered to the patient together in a single oral dosage composition such as a tablet or capsule, or each agent administered in separate oral dosage formulations. Where separate dosage formulations are used, the compounds of this disclosure and one or more additional active agents can be administered at essentially the same time, i.e., concurrently, or at separately staggered times, i.e., sequentially; combination therapy is understood to include all these regimens.

It will also be appreciated by those skilled in the art that in the process described herein the functional groups of intermediate compounds may need to be protected by suitable protecting groups. Such functional groups include hydroxy, amino, mercapto, and carboxylic acid. Suitable protecting groups for hydroxy include trialkylsilyl or diarylalkylsilyl (for example, t-butyldimethylsilyl, t-butyldiphenylsilyl or trimethylsilyl), tetrahydropyranyl, benzyl, and the like. Suitable protecting groups for amino, amidino and guanidino include t-butoxycarbonyl, benzyloxycarbonyl, or the like. Suitable protecting groups for mercapto include —C(O)—R″ (where R″ is alkyl, aryl or arylalkyl), p-methoxybenzyl, trityl or the like. Suitable protecting groups for carboxylic acid include alkyl, aryl or arylalkyl esters. Protecting groups may be added or removed in accordance with standard techniques, which are known to one skilled in the art and as described herein. The use of protecting groups is described in detail in Green, T. W. and P. G. M. Wutz, Protective Groups in Organic Synthesis (1999), 3rd Ed., Wiley. As one of skill in the art would appreciate, the protecting group may also be a polymer resin such as a Wang resin, Rink resin or a 2-chlorotrityl-chloride resin.

It will also be appreciated by those of skill in the art, although such protected derivatives of compounds of this disclosure may not possess pharmacological activity as such, they may be administered to a mammal and thereafter metabolized in the body to form compounds of this disclosure which are pharmacologically active. Such derivatives may, therefore, be described as “prodrugs”. In certain embodiments, compounds of this disclosure are in the form of a prodrug.

Furthermore, all compounds of this disclosure that exist in free base or acid form can be converted to their pharmaceutically acceptable salts by treatment with the appropriate inorganic or organic base or acid by methods known to those skilled in the art. Salts of the compounds of this disclosure can be converted to their free base or acid form by standard techniques.

In some aspects, the present disclosure provides methods for reducing the risk of metastatic spread of pancreas cancer cells, comprising (i) treating a subject with a neoadjuvant therapy, a tumor resection procedure, and optionally an adjuvant therapy when the subject has pancreas cancer cells that comprise a homozygous positive genotype for DPC4 or a single copy of DPC4, wherein the single copy is positive for DPC4, and that have a RUNX3 expression level that is increased as compared to normal cells, thereby reducing the risk of metastatic spread; or (ii) treating a subject with a tumor resection procedure, an adjuvant therapy, and optionally a neoadjuvant therapy when the subject has pancreas cancer cells that comprise a homozygous null genotype for DPC4 or have a single copy of DPC4, wherein the single copy is null for DPC4, and that have a RUNX3 expression level that is increased as compared to normal cells, thereby reducing the risk of metastatic spread. In certain embodiments, the subject of subpart (i) is treated with a neoadjuvant therapy comprising a systemic therapy and an adjuvant therapy comprising a systemic therapy.

In other aspects, the present disclosure provides methods for reducing the risk of local recurrence and/or proliferation of pancreas cancer, comprising treating a subject with a tumor resection procedure and optionally an adjuvant therapy when pancreas cancer cells from the subject comprise: (i) a heterozygous genotype for DPC4 and have a RUNX3 expression level that is similar as compared to normal cells from the subject; or (ii) a homozygous null genotype for DPC4 or have a single copy of DPC4, wherein the single copy is null for DPC4, and have a RUNX3 expression level that is increased as compared to normal cells from the subject, thereby reducing the risk of local recurrence and/or proliferation.

In yet other aspects, methods for treating pancreas cancer in a mammalian subject are provided. In certain embodiments, the methods comprise (a) requesting a test to determine (i) a genotype for DPC4 and (ii) an expression level of RUNX3, in pancreas cancer cells from the subject; and (b) administering to the subject a main therapy in combination with a neoadjuvant therapy, an adjuvant therapy or both, wherein the main therapy comprises tumor resection, and: (i) the neoadjuvant therapy comprises a systemic therapy when the pancreas cancer cells comprise a homozygous positive genotype for DPC4 or have a single copy of DPC4, wherein the single copy is positive for DPC4, and have a RUNX3 expression level that is increased as compared to normal cells from the subject, and optionally administering an adjuvant therapy comprising a systemic therapy; or (ii) the optional neoadjuvant therapy comprises a localized therapy when the pancreas cancer cells comprise a heterozygous genotype for DPC4 and have a RUNX3 expression level that is similar or decreased as compared to normal cells from the subject, and, and optionally administering an adjuvant therapy comprising a systemic therapy; or (iii) the adjuvant therapy comprises a systemic therapy when the pancreas cancer cells comprise a homozygous null genotype for DPC4 or have a single copy of DPC4, wherein the single copy is null for DPC4, and have a RUNX3 expression level that is increased as compared to normal cells from the subject, and optionally administering to the subject, prior to the main therapy, a neoadjuvant therapy comprising a localized therapy.

In a specific embodiment, a method comprises (a) requesting a test to determine (i) a genotype for DPC4 and (ii) an expression level of RUNX3, in pancreas cancer cells from the subject; and (b) the optional neoadjuvant therapy comprises a localized therapy when the pancreas cancer cells comprise a heterozygous genotype for DPC4 and have a RUNX3 expression level that is similar or decreased as compared to normal cells from the subject, and, and optionally administering an adjuvant therapy comprising a systemic therapy.

In certain other embodiments, methods for treating pancreas cancer in a mammalian subject comprise (a) requesting a test to determine a RUNX3 expression level and a Col6a1 expression level in pancreas cancer cells from the subject; and (b) administering to the subject a main therapy in combination with a neoadjuvant therapy, an adjuvant therapy or both, wherein the main therapy comprises tumor resection and: (i) the neoadjuvant therapy comprises a systemic therapy and an optional adjuvant therapy comprises a systemic therapy when the pancreas cancer cells have expression levels of both RUNX3 and Col6a1 that are increased as compared to normal cells from the subject; or (ii) an optional neoadjuvant therapy comprises a localized therapy and an adjuvant therapy comprises a systemic therapy when the pancreas cancer cells have a RUNX3 expression level that is increased as compared to normal cells from the subject and a Col6a1 expression level that is decreased or similar as compared to normal cells from the subject.

In another embodiment, methods for treating pancreas cancer based on a DPC4 genotype and RUNX3 expression level are provided, comprising administering to a subject a main therapy in combination with a neoadjuvant therapy, an adjuvant therapy, or both, wherein the main therapy comprises tumor resection, and (a) the neoadjuvant therapy comprises a systemic therapy and an optional adjuvant therapy comprises a systemic therapy when the pancreas cancer cells comprise a homozygous positive genotype for DPC4 or have a single copy of DPC4, wherein the single copy is positive for DPC4, and have a RUNX3 expression level that is increased as compared to normal cells from the subject; or (b) an optional neoadjuvant therapy comprises a localized therapy and the adjuvant therapy comprises a systemic therapy when the pancreas cancer cells are heterozygous for DPC4, and have a RUNX3 expression level that is similar to normal cells from the subject; or (c) an optional neoadjuvant therapy comprises a systemic therapy, and the adjuvant therapy comprises a systemic therapy, when the pancreas cancer cells from the subject comprise a homozygous null genotype for DPC4 or have a single copy of DPC4, wherein the single copy is null for DPC4, and have a RUNX3 expression level that is increased as compared to normal cells from the subject.

It will be appreciated that therapeutic regimens according to the present disclosure will vary according to information obtained regarding, e.g., DPC4 genotype, RUNX3 expression levels, Col6a1 expression levels, and Spp1 expression levels. For example, in certain embodiments, a therapeutic regimen comprises tumor resection and an optional adjuvant therapy if the pancreas cancer cells from the subject comprise (i) a heterozygous genotype for DPC4 and have a RUNX3 expression level that is similar as compared to normal cells from the subject; or (ii) a homozygous null genotype for DPC4 or have a single copy of DPC4, wherein the single copy is null for DPC4, and have a RUNX3 expression level that is increased as compared to normal cells from the subject. In certain other embodiments, a subject having pancreas cells comprising (i) or (ii) above may be treated with tumor resection, but not an optional adjuvant therapy if, for example, the risk of local recurrence and/or proliferation is determined to be low. In certain embodiments, a therapeutic regimen comprises a neoadjuvant therapy comprising a systemic therapy; a main therapy; and an adjuvant therapy comprising a systemic therapy when the pancreas cancer cells comprise a homozygous positive genotype for DPC4 or have a single copy of DPC4, wherein the single copy is positive for DPC4, and have a RUNX3 expression level that is increased as compared to normal cells from the subject. In alternative embodiments, under such circumstances an adjuvant therapy not included in the regimen.

In certain embodiments, both a neoadjuvant therapy comprising a localized therapy and an adjuvant therapy comprising a systemic therapy are optional, and resection is the only necessary therapy, when the pancreas cancer cells comprise a heterozygous genotype for DPC4 and have a RUNX3 expression level that is similar or decreased as compared to normal cells from the subject.

In certain embodiments, a therapeutic regimen comprises a neoadjuvant therapy comprising a localized therapy; a main therapy; and an adjuvant therapy comprising a systemic therapy when the pancreas cancer cells comprise a homozygous null genotype for DPC4 or have a single copy of DPC4, wherein the single copy is null for DPC4, and have a RUNX3 expression level that is increased as compared to normal cells from the subject. In alternative embodiments, under such circumstances a neoadjuvant therapy is not included in the regimen.

In certain embodiments, a therapeutic regimen comprises a neoadjuvant therapy comprising a localized therapy; a main therapy; and an adjuvant therapy comprising a systemic therapy when the pancreas cancer cells comprise a homozygous null genotype for DPC4 or have a single copy of DPC4, wherein the single copy is null for DPC4, and have a RUNX3 expression level that is increased as compared to normal cells from the subject. In alternative embodiments, under such circumstances a neoadjuvant therapy is not included in the regimen, thereby indicating that the subject would benefit from a tumor resection procedure and optionally an adjuvant therapy to reduce the risk of local recurrence and/or proliferation.

Thus, in some embodiments, a therapeutic regimen comprises a neoadjuvant therapy comprising chemotherapy; a main therapy; and an adjuvant therapy comprising chemotherapy when the pancreas cancer cells comprise a homozygous positive genotype for DPC4 or have a single copy of DPC4 that is positive for DPC4 and have a RUNX3 expression level that is increased as compared to normal cells from the subject. Under these circumstances, the cells have an elevated metastatic potential, but local tumor growth is less of a concern.

In other embodiments, a therapeutic regimen comprises a therapeutic regimen comprises a main therapy comprising resection; and an adjuvant therapy comprising radiation therapy when the pancreas cancer cells comprise a heterozygous genotype for DPC4 and a level of RUNX3 expression that is similar or decreased as compared to normal cells from the subject. Under these circumstances, the cells have a high tumor growth potential but a low metastatic potential; thus, it is imperative to remove the cancerous tissue from the pancreas and preventing metastatic spread is a lesser concern.

In yet further embodiments, a therapeutic regimen comprises a main therapy comprising resection; and an adjuvant therapy comprising chemotherapy and radiation therapy when the pancreas cancer cells comprise a homozygous null genotype for DPC4 or have a single copy of DPC4 that is null for DPC4, and have a RUNX3 expression level that is increased as compared to normal cells from the subject. In an alternative embodiment, under such circumstances the main therapy (resection) may be replaced by a short course of radiation therapy followed by chemotherapy. Under these circumstances, both tumor growth potential and metastatic potential are significant concerns. However, resection may be administered first due to the concern that a delay to administer a neoadjuvant therapy (i.e., chemotherapy and/or radiation therapy) might allow for fatal local advancement of the pancreas cancer.

In any of the aforementioned embodiments, therapy regimens (neoadjuvants and adjuvants) of this disclosure can include expression or activity inhibitors of a cyclin D, a cyclin E, or a combination thereof, or in combination with conventional chemotherapies, such as gemcitabine, nab-paclitaxel, FOLFIRINOX combination, or any combination thereof. In yet other embodiments, therapy regimens of this disclosure (neoadjuvants and adjuvants) include an agent capable of degrading hyaluronic acid (HA), such as hyaluronidases, a CD44 antagonist, or any combination thereof.

Additional information, such as the location or appearance of the pancreas cancer cells or pancreas cancer precursor lesions within the pancreas, the overall health of the subject, whether the subject exhibits other cancer biomarkers (e.g., a deleterious mutation in the p53 gene), and other factors may influence a choice of therapeutic regimen.

### EXAMPLES

### Example 1

### Experimental Procedures

### Mouse Strains

All animal studies were approved by the Institutional Animal Care and Use Committee of the Fred Hutchinson Cancer Research Center. Conditional Trp53^{LSL-R172H/+}, Dpc4^{flox/+}, Kras^{LSL-G12D/+}, and p48^{cre/+} mice strains described earlier (Hingorani et. al., 2003; Hingorani et. al., 2005; Izeradjene et al., 2007) were interbred to obtain Kras^{LSL-G12D/+}; Trp53^{LSL-R172H/+}; Dpc4^{+/+}; p48^{Cre/+} (KPC), Kras^{LSL-G12D/+}; Trp53^{LSL-R172H/+}; Dpc4^{flox/+}; p48^{Cre/+} (KPDC), Kras^{LSL-G12D/+}; Trp53^{LSL-R172H/+}; Dpc4^{flox/flox}; p48^{Cre/+} (KPDDC) and various littermate control animals on a mixed background. Nude mice (Athymic NCr-nu/nu, NCI Frederick USA) and NOD.SCID/NCr mice (CCEH Specialized Mouse Services, FHCRC) were used in pulmonary metastasis assays.

### Histology and Immunohistochemistry

Tissues were fixed in 10% neutral buffered formalin, embedded in paraffin and 5 μm sections were prepared. Routine H&E and immunohistochemistry (IHC) were performed as previously described (Hingorani et. al., 2003) and IHC scoring was done blinded. Biotinylated secondary antibody and the Elite vectastain kit (Vector Lab, CA, USA) were used for signal detection and DAB was used as a chromogen substrate. Primary antibodies: amylase (1:800, Sigma, Chicago, Ill., USA); CK-19 (DakoCytomation, Denmark); cleaved caspase 3 (1:100, Biocare, CA, USA); Dpc4 (1:300, Abcam, MA, USA); insulin (1:200, Dako, CA, USA); Ki-67 (1:25, Dako, CA, USA); and Runx3 (1:200 for murine and 1:100 for human IHC, Cell Signaling, MA, USA).

### Assessment of Primary and Metastatic Disease Burden

Complete necropsies were performed on all study animals. Gross pathology and histopathology of all internal organs were analyzed for macroscopic and microscopic disease, respectively. Primary (1°) histology was defined as representing ≥50% assessed tissue; secondary (2°) histology involved >25% but <50%.

### Statistical Analyses

Unless otherwise indicated, the significance of data was determined by the Student's t-test (two-tailed) for all in vitro studies. A value of p<0.05 was deemed significant. Fisher's exact t-test was performed for comparison of metastatic disease burdens in KPC vs. KPDC cohorts. All statistical analyses were performed using GraphPad Prism 6.0 software.

### Immunoblots

Murine primary PDA cells were prepared as described previously (Hingorani et al., 2005) and used at low passage number (less than 15 for all assays). Human PDA cell lines (AsPc1, BxPc3, CAPAN2, CFPAC-1, HPAFII, MiaPaCa-2, Panc-1 and Su86.86) were a gift from Translational Genomics (Phoenix, Ariz., USA). All cells were grown in DMEM/F12 supplemented with 10% FBS, 0.5% Glucose, and 1× Pen/Strep (100 U/ml/100 μg/ml). Whole cell lysates were prepared in 1×RIPA buffer (Millipore, CA, USA) supplemented with 1× protease inhibitor (Sigma, MO, USA) and phosphatase inhibitor cocktails I and II (Sigma, MO, USA). For immunoblot experiments, 20 μg (murine) or 30 μg (human) total protein from whole cell lysates were separated on a 4-12% Bis-tris gel system (Invitrogen, CA, USA) and transferred to PVDF membrane (Millipore, CA, USA). Primary antibodies used were Actin, Col6a1, Dpc4, Flag, Gapdh, p16, p19, p21, Parp and Spp1 (Santa Cruz, Calif., USA); p53 (Vector Lab, CA, USA); and Runx3 (Cell Signaling, MA, USA).

### Cell Fractionation

Nuclear and cytoplasmic fractions of murine PDA cells were prepared using the Nuclear Extract Kit from Active Motif (Carlsbad, Calif., USA). Approximately 20 μg of protein from nuclear or cytoplasmic cell fractions were loaded on 4-12% Bis-Tris gels and probed with Runx3, Parp and Gapdh antibodies (Parp and Gapdh were used as nuclear and cytoplasmic markers, respectively).

RNA Isolation and qRT-PCR

High-quality total RNA was extracted from cells grown to −80% confluency using TRIzol reagent (Invitrogen, CA, USA), treated with DNase (Applied Biosystems, CA, USA) and further purified using the RNeasy kit (Qiagen, Germany). RNA was quantified using a NanoDrop. Murine TGFβ/BMP signaling pathway (PAMM-035; with modifications to include primer sets for all Runx family members) and mouse extracellular matrix (PAMM-013) specific RT² profiler PCR arrays were obtained from SA Biosciences Co. (Frederick, Md., USA). For cDNA synthesis, total RNA was reverse transcribed using RT² first strand kit (SA Biosciences). Array analyses with SYBR Green PCR Master Mix (SA Biosciences) were carried out according to the manufacturer's protocol on a Bio-Rad CFX-96 PCR System (Bio-Rad, VA, USA). Gene expression differences were determined using the 2^{-ΔΔCt} method following standard protocols (SA Biosciences). Total RNA was reverse transcribed using a high-capacity cDNA synthesis kit (Applied Biosystems, CA, USA) and qRT-PCR was performed using SYBR green supermix (Bio-Rad, VA, USA) on a CFX-96 thermal cycler. PCR reactions included initial denaturation at 95° C. for 5 minutes, followed by 40 cycles of incubation at 95° C. for 30 seconds and 60° C. for 1 minute. Melting curves were generated for each reaction to exclude false positive signals due to primer dimers. CPHA (Cyclophilin A) was used as an internal control. RUNX3 transcript levels in human cell lines were analyzed by the Taq-man assay per the manufacturer's recommendations (Applied Biosystems, CA, USA) with β-actin as an internal control.

### Proliferation, Morphology, and Migration Assays

For murine and human cell proliferation assays, equal numbers of cells (30,000) were plated in triplicate for each cell line and incubated in the presence or absence of TGFβ (5 ng/ml, R&D Systems, USA). Cells were counted manually at 24 hour intervals. Data points represent the mean±SEM of triplicate determinations from at least 3 independent experiments. For migration assays, cells were first incubated in the presence of TGFβ (5 ng/ml) for 60 hours to achieve growth arrest in order to separate out the effects of proliferation into rather than migration across a subsequently introduced wound (see Izeradjene et al., 2007). Confluent cultures were then serum starved for 8 hours and scratched with a 20 μl pipette tip to generate three horizontal wounds per well. All experiments were conducted in triplicate at least 3 independent times on at least 2 different cell lines from each genetic background. A minimum of three points across each wound were assessed independently by two investigators at the indicated time intervals for the presence of cells within the expanse. Representative photographs were taken from a minimum of 9 separate high power fields. For all assays, media (1% serum) was replaced every 24 hours with or without TGFβ as indicated and wells were photographed every 24 hours. All migration assays were quantified using ImageJ (NIH) with Scratch Assay V plugin (Netherland Cancer Institute, Netherlands). Results represent means±SEM of triplicate wells from three independent experiments such that at least 50-70 points for each condition were quantified. Some human PDA cell migration assays were also performed using μ-Dish 35 mm cell culture inserts (Ibidi, WI, USA).

### Immunofluorescence

Cells were grown on glass cover slips and treated for 72 hours in the presence or absence of TGFβ (5 ng/ml); media was replaced every 24 hours. For E-cadherin immunofluorescence, cells were fixed in ice cold methanol:acetone (1:1) for 15 minutes. For F-actin IF, cells were fixed in 4% paraformaldehyde for 30 minutes at RT followed by brief rinse in 0.15M glycine to quench the PFA. The cells were then washed with PBS, blocked for 1 hour at RT (2% fatty acid-free BSA/2.5% goat serum) and incubated with E-cadherin primary antibody (1:200, BD Biosciences) or Alexafluor 488 F-actin (1:1000) for 1 hour at RT. Alexafluor 488 secondary antibody was used to visualize E-cadherin antibody. Cells were counterstained with DAPI and the slips mounted with Prolong Gold Anti-fade (Invitrogen, CA, USA). Images were acquired using a Nikon Eclipse 80i microscope with NIS Elements Software (Nikon, v3.1) and analyzed using ImageJ software (NIH).

### Invasion Assays

Diluted Matrigel (BD Biosciences) was added to the upper chamber of 24-well transwell plates and incubated at 37° C. for at least 4 h. Cells were trypsinized, washed 3 times with culture media containing 1% FBS and resuspended to a density of 10⁶ cells/ml. The Matrigel plugs were gently washed with serum-free culture media and 100 μl of cell suspension added on top. The lower chamber of the transwell was filled with 600 μl of culture media containing 5 μg/ml fibronectin as an adhesive substrate and the plates were incubated at 37° C. for 24 h. Transwells were removed, stained with 0.5% Toluidine Blue in 2% Na₂CO₃ and non-invaded cells were scraped off the top of the transwell. Invaded cells were imaged under a light microscope and quantified with ImageJ software (NIH).

### Soft Agar Assays

Trypsinized cells were added to a dissolved noble agar solution (1.2% in DMEM/10% FBS/1% Pen/Strep at 37° C.) and further diluted with basic growth media to a concentration of 2.5×10³ cells/ml and 0.27% agar. The cell suspension was plated in triplicate (3 ml) in 35 mm plates and colonies counted after 2-3 weeks of growth.

### Pulmonary Metastasis Assays

At least 3 nude (Athymic NCr-nu/nu) animals were injected via tail vein for each independently derived murine cell line (1×10⁵ or 1×10⁶ cells/injection depending on the experiment) and all experiments were performed at least twice. Human PDA cells (1×10⁶ cells/injection) were similarly injected into NOD.SCID animals. Animal health was monitored daily and all study animals in a given cohort were sacrificed when any one of the animals in that particular group showed signs of cachexia, lethargy or respiratory distress. Metastatic disease burdens were assessed as a ratio of cross-sectional area occupied by tumor to whole lung area using ImageJ software (NIH).

### Runx3, Dpc4 and Col6a1 Cloning

Full length mouse Runx3 and Dpc4 were obtained from Open Biosystems (Huntsville, Ala., USA) and subcloned into a pMSCV Puro IRES GFP vector (Addgene, MA, USA). Human RUNX3 and COL6A1 were similarly cloned into pBABE hygromycin vectors. Fidelity of the cloned genes was confirmed by sequencing. High-titer retrovirus was made in HEK293T cells using a standard protocol (Addgene, MA, USA). Cells were selected with the appropriate antibiotic for at least 5 days or until all uninfected cells were dead. Transgene expression was confirmed by immunoblot using Flag tag-specific and protein-specific antibodies.

### RNA Interference in Murine and Human PDA Cells

shRNA sequences targeting murine Runx3, Dpc4 and Col6a1 in pLKO.1 vectors and for human RUNX3 were procured in pGIPZ vectors (Open Biosystems). PDA cells were infected with high-titer lentivirus and selected for stable expression of the specific shRNA. Knockdown was confirmed by qRT-PCR and/or immunoblot.

### Runx3 Luciferase Reporter Assays

The full length murine Runx3 promoter (containing 3 putative Runx3 binding sites) was cloned into the pMCS-Red Firefly Luciferase vector (Thermo Scientific, IL, USA) and co-transfected with increasing concentrations of Flag-Runx3 plasmid into HEK293T cells. Cell lysates were prepared at 48 hours in reporter lysis buffer (Promega WI, USA), substrate added and luciferase activity recorded on a luminometer (Veritas, WI, USA). Results represent means±SEM of two independent transfections each assayed in duplicate. The entire experiment was repeated twice with similar results.

### Spp1 ELISA

Blood was collected from mice at necropsy by cardiac puncture into heparin-charged syringes and centrifuged at 8,000 rpm in a tabletop centrifuge. The supernatant (plasma) was recovered, aliquoted and immediately frozen at −80° C. Aliquots were thawed on ice, diluted 1000-fold in diluent, and assays performed according to manufacturer's instructions in the Spp1 Quantikine ELISA kit (R&D Systems, USA). Plates were read at 450 nm (with wavelength correction at 540 nm) on a SpectraMax M5 plate reader (Molecular Devices, CA, USA). Samples were analyzed in duplicate and evaluated against a standard curve.

### Targeted ICGC Data Analyses

Gene array-based sequencing data and associated clinical data were downloaded from the ICGC data portal (data release 17; see webpage dcc.icgc.org). Relative array-based expression levels of RUNX3, COL6A1 and SPP1 were compared to survival and/or relapse pattern for all Stage II pancreas cancer patients who underwent surgical resection and for whom expression arrays were performed.

### Human Tissue Microarray (TMA) and Quantification of Immunohistochemistry

A tissue microarray was prepared from 108 patients who underwent resection for PDA. Survival data and adequate tissue for immunohistochemical analyses were available for 88 of the patients. Each patient tumor was represented by two independent cores on the array. Each TMA slide also contained portions of duodenum with Peyer's patches which served as an internal positive control for RUNX3. Immunohistochemical labeling for RUNX3 was performed as described above and the slides were independently evaluated in a blinded manner. In the event of discordant scores, the IHC was reviewed and a consensus reached. Intensity of nuclear RUNX3 expression in tumor epithelial cells was scored in a semi-quantitative fashion on a four-point scale as follows: zero (no staining), 1+(weak), 2+(moderate), and 3+(strong). The highest score for each pair of tumor samples was used in survival and treatment response analyses. Lymphocytes served as an internal positive control for each tissue section and were readily distinguished from the epithelium based on morphology and nuclear/cytoplasmic ratio. Survival curves were plotted using the Kaplan-Meier method to compare cumulative probability of survival on the basis of RUNX3 expression and significance was assessed by the Wilcoxon test.

### In Silico Analyses

In silico analyses of various gene promoter regions for putative Runx3 binding sites and SBE (SMAD binding elements) were performed with publicly available software www.cbrc.jp/research/db/TFSEARCH.html. Regions up to −1500 bp upstream of the transcription start site were examined.

Quantitative PCR primers

| Gene | Forward | Reverse |
|---|---|---|
| m Runx3 | agagtttcacgctcacaatc (SEQ ID NO.: 1) | ggagaaggggttcaggtc (SEQ ID NO.: 2) |
| m Col6a1 | ctgctgctacaagcctgct (SEQ ID NO.: 3) | ccccataaggtttcagcctca (SEQ ID NO.: 4) |
| m Cpha | GAGCTGTTTGCAGACAAAGTTC | CCCTGGCACATGAATCCTGG |
| | (SEQ ID NO.: 5) | (SEQ ID NO.: 6) |
| m SPP1 | TTTCACTCCAATCGTCCCTACA | TCAGTCCATAAGCCAAGCTATCA |
| | (SEQ ID NO.: 7) | (SEQ ID NO.: 8) |
| H COL6A1 | ACACCGACTGCGCTATCAAG | CGGTCACCACAATCAGGTACTT |
| | (SEQ ID NO.: 9) | (SEQ ID NO.: 10) |
| H CPHA | CCCACCGTGTTCTTCGACATT | GGACCCGTATGCTTTAGGATGA |
| | (SEQ ID NO.: 11) | (SEQ ID NO.: 12) |
| H SPP1 | CTCCATTGACTCGAACGACTC | CAGGTCTGCGAAACTTCTTAGAT |
| | (SEQ ID NO.: 13) | (SEQ ID NO.: 14) |

m, murine; H, human; * TaqMan assay probes (Applied Biosystems) were used for human RUNX3 qRT-PCR.

shRNA sequences (sense strands)
m Runx3:
(SEQ ID NO: 15)
TAGAGAGGACATTGATTTGGG
m Col6a1:
(SEQ ID NO: 16)
TACACAAAGCAAAGAGTGTGC
H RUNX3:
(SEQ ID NO: 17)
CGGCAGAAGCTGGAGGACC

### Example 2

### Results

### Concomitant Trp53 Mutation Restores PanIN-to-PDA Progression in the Context of Kras^{G12D} Expression and Dpc4 Deletion

Conditional endogenous expression and deletion of critical genes (FIG. 8A) were targeted to the developing murine pancreas using strategies described previously (Hingorani et. al., 2003; Hingorani et. al., 2005; Izeradjene et al., 2007). We first established that animals with targeted pancreatic expression of point-mutant p53 and concomitant heterozygous deletion of Dpc4/Smad4, in the absence of oncogenic Kras^{G12D}, developed and aged normally with unperturbed pancreatic parenchymal architecture and intact exocrine and endocrine synthetic functions (FIG. 8B).

To investigate the effects of concomitant p53 and Dpc4 mutation on neoplasms initiated by oncogenic Kras, Kras^{LSL-G12D/+}; Trp53^{LSL-R172H+}; Dpc4^{flox/+}; p48^{Cre/+} (KPDC) quadruple mutant mice were generated and aged until symptomatic. Concomitant heterozygous deletion of Dpc4 shortened survival compared with Kras^{LSL-G12D/+}; Trp53^{LSL-R172H/+}; p48^{Cre/+} (KPC) littermates (FIG. 1A). KPDC animals developed solid, multinodular tumors similar to those of KPC mice (FIGS. 1B and 1C), albeit much larger (FIGS. 1D and 1E). Histologic progression in KPDC mice followed the classical PanIN-to-PDA and not MCN-to-PDA sequence. Low-grade PanIN were seen in young animals and subsequently progressed to higher grade and then to invasive PDA (FIGS. 1F-1I). CK-19 expression (FIG. 1J) confirmed the ductal epithelial nature of these neoplasms, and alcian blue revealed their abundant cytoplasmic mucin (FIG. 1K). The histologic morphology was predominantly glandular (Table 1) and other less common histologies seen in human PDA, including anaplastic and sarcomatoid, were similarly rare in KPDC tumors.

TABLE 1
Clinical Spectrum of Disease in KPDC Mice

| | | | Histology | | Metastases | | | |
|---|---|---|---|---|---|---|---|---|
| ID | Age | PDA | 1⁰ | 2⁰ | Liver | Lung | Ascites | Cachexia |
| 1 | 151 | Y | G | | Y^{m} | N | Y | N |
| 2 | 169 | Y | G | | N | N | Y | Y |
| 3 | 125 | Y | G | | Y^{m} | Y^{m} | Y | Y |
| 4 | 172 | Y | G | U | Y^{M} | N | N | Y |
| 5 | 165 | Y | G | | N | N | N | Y |
| 6 | 329 | Y | G | U | Y^{M} | Y^{M} | N | N |
| 7 | 120 | Y | G | U | N | N | * | * |
| 8 | 193 | Y | G | C | N | N | Y | Y |
| 9 | 180 | Y | G | U | Y^{m} | N | Y | Y |
| 10 | 95 | Y | G | U | Y^{m} | N | N | Y |
| 11 | 190 | Y | G | | Y^{M} | Y^{m} | Y | N |
| 12 | 211 | Y | G | U | N | N | N | N |
| 13 | 98 | Y | G | | N | N | N | Y |
| 14 | 172 | Y | G | C; U | Y^{M} | N | Y | Y |
| 15 | 108 | Y¹ | G | | N | N | N | N |
| 16 | 178 | Y | G | | N | N | N | N |
| 17 | 78 | Y | G | U | N | N | N | N |
| 18 | 206 | Y | G | | N | N | N | Y |
| 19 | 181 | Y | G | | N | N | N | N |
| 20 | 229 | Y | G | U | Y^{m} | N | N | N |
| 21 | 162 | Y | G | | N | N | N | N |
| 22 | 120 | Y | G | | N | N | Y | Y |
| 23 | 102 | Y | G | | N | N | N | N |
| 24 | 211 | Y | G | | N | N | N | N |
| 25 | 153 | Y | G | U | Y^{m} | Y^{m} | N | Y |
| 26 | 143 | Y | G | | Y^{M} | Y^{M} | Y | Y |
| 27 | 189 | Y | G | | Y^{M} | Y^{m} | N | Y |
| 28 | 132 | Y | G | U | N | N | N | Y |

Y, disease present; N, no disease detected; G, glandular; U, undifferentiated; ^{M}macroscopic; ^{m}microscopic; *, not evaluable secondary to necrosis; ¹focally invasive PDA but animal succumbed to thymoma; C, cystic.

Haploinsufficiency of Dpc4 Attenuates the Metastatic Drive of Kras^{G12D} and Trp53^{R172H}

Belying their earlier demise, KPDC animals manifested an unexpected and significant decrease in metastatic disease. Their KPC littermates developed widely disseminated disease as described previously (Hingorani et al., 2005), with a heavy macroscopic tumor burden, principally to the liver (53%) and lungs (47%) (FIG. 1L). In contrast, only 21% of KPDC littermates developed macroscopically evident metastases which were largely confined to the liver (FIG. 1L and Table 1). Moreover, as most metastases in KPDC mice were microscopic, they did not contribute substantively to overall tumor burden. Thus, the evolution of pancreas cancer in KPDC mice is shifted toward a higher primary tumor burden and less metastatic disease.

### Genetic Progression of KPDC PDA

To investigate the basis for the distinct in vivo biological behavior of KPDC PDA, primary ductal carcinoma cells were purified as previously described (Hingorani et al., 2005; Schreiber et al., Gastroenterol. 127: 250, 2004). First, confirmation that the purified PDA cells had recombined and activated the conditional Kras^{LSL-G12D} and Trp53^{LSL-R172H} alleles, and likewise deleted the Dpc4 allele (FIGS. 8C-8E) we performed. Disease progression was invariably accompanied by spontaneous LOH of Trp53 (FIG. 8D), as occurs in murine (Hingorani et al., 2005) and human (Scarpa et al., Am. J. Pathol. 142:1534, 1993) PDA with point mutations in one TP53 allele. Invasive KPDC PDA cells typically did not undergo LOH of Dpc4, but instead frequently retained the remaining wild type (WT) allele (FIG. 8E) and persistent protein expression (FIG. 1M), unlike the spontaneous loss or epigenetic silencing of the second allele that occurs in KDC mice during disease progression (Izeradjene et al., 2007). Detectable levels of Trp53 were observed consistent with point-mutant expression and LOH. Cdkn2a/Ink4a (p16) expression was frequently lost, although that of the contiguous p19 allele was retained, indicating specific promoter methylation rather than genomic deletion as the mechanism of silencing (Izeradjene et al., 2007). The loss of p16 is notable as prior studies of both KPC (Hingorani et al., 2005) and Kras^{LSL-G12D/+}; p16/p19^{-/-}; Cre Bardeesy et al., Proc. Nat'l. Acad. Sci. U.S.A. 103:5947, 2006) animals found no additional loss of other major TSG during disease progression, establishing non-overlapping mutational spectra in the two model systems. Thus, heterozygous mutation of Dpc4 increased the selection pressure to lose p16, underscoring that WT levels of Dpc4 could be tumor promoting in this context and that the haploinsufficiency of Dpc4 in inhibiting primary tumor growth also attenuated metastatic potential. The spontaneous progression of PDA with Kras^{G12D}, Trp53^{R172H}, Dpc4^{+/-} and loss of p16 encompassed all four cardinal mutations seen in human PDA (Iacobuzio-Donahue et al., Clin. Cancer Res. 18:4257, 2012; Iacobuzio-Donahue et al., 2000).

Heterozygous Loss of Dpc4 Inhibits Metastatic Behavior Induced by Kras^{G12D} and Trp53^{R172H}

Functional assays were performed to explore the relative metastatic deficiency of KPDC pancreas cancers. First, the epithelial-to-mesenchymal transition (EMT), a program of morphological and molecular changes implicated in the metastatic phenotype, was observed to be qualitatively intact in KPDC PDA cells. KPDC cells developed actin stress fibers in response to TGFβ (FIGS. 2A and 9A) and down-regulated surface expression of E-cadherin (FIGS. 2B and 9B). However, they were less likely to separate from each other in response to TGFβ than KPC cells and, despite their qualitative ability to undergo EMT, both the basal migration and invasion of KPDC cells were significantly impaired (FIGS. 2C-2E, 9C and 9D). Moreover, whereas TGFβ stimulated KPC cells to migrate, KPDC cell migration was instead either unaffected or further inhibited. KPC cells in monolayer culture also readily formed foci in response to TGFβ, whereas KPDC cells did not (data not shown).

The cellular phenotypes could be interconverted by depleting or overexpressing Dpc4 in KPC or KPDC cells, respectively. Dpc4 depletion in KPC cells inhibited their migration and overexpression of Dpc4 in KPDC cells increased theirs (FIGS. 9E-G). After injection into the circulation, KPC cells formed numerous large pleural and parenchymal metastases sufficient to induce respiratory failure, reflecting the metastatic aggressiveness seen in their autochthonous counterpart (FIGS. 2G and 2H). KPDC cells formed far fewer and smaller lung metastases in animals that remained clinically robust at comparable time points (FIGS. 2F and 2H).

Collectively, these studies reveal that both the baseline properties and responses to TGFβ are essentially mirror images in the presence of WT vs. heterozygous loss of Dpc4.

### Runx3 Drives Metastasis in PDA

The dramatically different behaviors of KPC and KPDC carcinomas suggested that comparative expression profiling might provide mechanistic insights. Expression analyses using customized TGFβ-signaling-specific qPCR arrays identified 15 genes that were upregulated at least 2-fold in KPC vs. KPDC cells and that might, therefore, represent metastasis-promoting genes (Table 2). Dpc4 expression was approximately 2-fold higher in KPC than KPDC cells, confirming the fidelity of the array results. Completely distinguishing itself was Runx3, which was upregulated 36-fold in KPC cells (Table 2). Runx3 belongs to a family of runt-related transcription factors involved in development and differentiation of which Runx1 and Runx2 are the best characterized (Blyth et al., Nat. Rev. Cancer 5:376, 2005). The specificity of Runx3 upregulation was underscored by the lack of significant changes in the other family members (1.2-fold increase for Runx1 and 1.5-fold for Runx2; not shown). RUNX1 and RUNX2 have been identified as TSG in hematopoietic malignancies. Its alternate designation as acute myelogenous leukemia 2 (AML2) notwithstanding, what little is known about the role of RUNX3 in malignancy comes primarily from studies of solid tumors, where it has been implicated on both sides of the cancer divide (i.e., as a TSG in some reports and an oncogene in others). Indeed, its specific role in tumorigenesis remains the subject of considerable controversy (see, for example, Ito et al., Oncogene 28:1379, 2009; Levanon et al., EMBO Mol. Med. 3:593, 2011; Levanon et al., Mechanisms Dev. 109:413, 2001; Li et al., Cell 109:113, 2002, and see below).

TABLE 2
Focused TGFβ pathway PCR array
analysis in KPC vs. KPDC PDA cells

| Gene | Fold Change |
|---|---|
| Runx3 | 36.0541 |
| Dlx2 | 7.4322 |
| Tgfbi | 4.6685 |
| Id1 | 3.1935 |
| Itgb5 | 3.1581 |
| Ltbp2 | 3.1563 |
| Ltbp1 | 3.0789 |
| Bmper | 2.997 |
| Tgfb1i1 | 2.9623 |
| Itgb7 | 2.6516 |
| Bmp1 | 2.4641 |
| Smad4 | 2.3829 |
| Tgfb3 | 2.3613 |
| Smad5 | 2.1156 |
| Bmpr1a | 2.0592 |

Genes upregulated at least 2-fold in KPC compared to KPDC carcinoma cells. Values are expressed as fold change (n=3 each).

Runx3 gene expression and protein levels correlated directly with metastatic potential (FIGS. 3A and 3B). Specific IHC for Runx3 revealed no discernible expression in normal pancreatic ducts, islets or acini; scattered lymphocytes, common sites of elevated Runx3 and useful internal controls (Yarmus et al., Proc. Nat'l Acad. Sci. U.S.A. 103:7384, 2006), showed strong nuclear expression (FIG. 3C). Murine KPC tumors had moderate-to-strong nuclear Runx3 expression (FIGS. 3D and 3E), whereas KPDC tumors typically showed very weak expression (FIGS. 3F and 3G). Preinvasive lesions adjacent to invasive KPC carcinomas also lacked Runx3 expression (FIG. 3H). KPC metastases exhibited strong nuclear Runx3 expression (FIG. 3I) and, in KPDC mice that did bear metastases, both these and the primary tumors often expressed high levels of Runx3 (FIGS. 3J-L). Human tissue samples representing normal and pancreatitis conditions showed no discernible RUNX 3 expression, and RUNX3 expression was rare in pancreatic neuroendocrine tumors (PNETs). PNETs, also referred to as “islet cell tumors”, arise from endocrine and neuronal cells within the pancreas. In contrast, RUNX3 was frequently overexpressed in PDAs (FIG. 14). These data indicate that RUNX3 overexpression is specific to PDAs and is not associated with normal, inflamed (pancreatitis), or non-PDA neoplastic (PNET) conditions of the pancreas. The upregulation of Runx3 as KPC PanIN become invasive and metastatic coincides with the apparent requirement for LOH of p53 and subsequent accumulation of the point-mutant protein (Hingorani et al., 2005). These observations implicate WT p53—reported to complex with RUNX3 (Yamada et al., J. Biol. Chem. 285:16693, 2010)—in suppressing Runx3 or point-mutant p53 in stabilizing Runx3.

### Runx3 Promotes Metastatic Seeding and Colonization by Remodeling the ECM

To further investigate its effects on metastatic potential, Runx3 expression in independent KPDC and KPC carcinoma cell lines was varied (data not shown). Runx3 overexpression greatly increased the basal migration of KPDC cells, and silencing Runx3 inhibited KPC cell migration (FIGS. 3M, 10A and 10B). TGFβ stimulated migration only in the context of high Runx3 expression. Anchorage-independent growth in soft agar was similarly influenced by changes in Runx3 expression (data not shown). KPDC-Runx3 cells also readily formed lung metastases in immunocompromised mice (FIGS. 3N and 3O), an ability virtually absent in control-transfected KPDC cells and once again more like baseline KPC cells. A trend toward reduced lung metastasis after Runx3-knockdown in KPC cells was also observed but did not reach statistical significance, perhaps reflecting incomplete Runx3 silencing (FIGS. 3P and 3Q).

KPDC cells were unable to efficiently seed metastases even after direct inoculation into the bloodstream. To successfully colonize a distant site, disseminated cells must not only intravasate but also survive in the circulation, extravasate in a target organ, and fashion a niche that supports metastatic growth. Therefore, a focused array profiling for ECM genes comparing the highly metastatic KPC and non-metastatic KPDC PDA cells was performed. A survey of the top differentially expressed genes revealed several that have been previously implicated in critical ECM functions, promoting local invasiveness and remodeling of the metastatic niche (Psaila and Lyden, Nat. Rev. Cancer 9, 285, 2009) (Table 3). These include matrix Mmps, Timps, Versican, Spp1 and Sparc (Lunardi et al., Cancer Lett. 343:147, 2014).

TABLE 3
Focused ECM PCR Array Analysis in
KPC vs. KPDC PDA cells

| Gene | Fold Change |
|---|---|
| Col6a1 | 12.5194 |
| Sparc | 8.3402 |
| Vcan | 7.6945 |
| Spp1 | 7.1535 |
| Timp3 | 6.9182 |
| Mmp10 | 6.4396 |
| Cdh2 | 4.3222 |
| Tgfbi | 4.2408 |
| Fbln1 | 3.557 |
| Thbs1 | 3.3539 |
| Mmp2 | 3.1125 |
| Adamts1 | 3.0894 |
| Ecm1 | 2.9625 |
| Vcam1 | 2.8629 |
| Fn1 | 2.7477 |
| Sgce | 2.7383 |
| Mmp11 | 2.7008 |
| Thbs3 | 2.5626 |
| Ncam1 | 2.4502 |
| Col5a1 | 2.0393 |
| Timp2 | 2.0159 |
| Selp | 2.013 |
| Ctgf | 1.9957 |

Genes upregulated at least 2-fold in KPC compared to KPDC carcinoma cells. Values are expressed as fold change (n=3 each).

Osteopontin (SPP1) is upregulated in human PDA and portends poor prognosis (Poruk et al., Pancreas 42:193, 2013). Spp1 protein (FIG. 4A) and transcript levels (Table 3) are also significantly upregulated in KPC versus KPDC PDA cells. Two consensus RUNX3-binding sites and two SMAD-binding elements in both the human SPP1 and murine Spp1 promoter regions were identified, and found that the promoter responds to RUNX3 overexpression (FIG. 11A). Overexpression or silencing of Runx3 in KPDC or KPC cells, respectively, caused corresponding changes in Spp1 expression (FIGS. 4B, 4C, 11B and 11C). As Spp1 is secreted, we therefore tested whether exogenously added Spp1 influenced migration. KPDC cell migration increased significantly in the presence of Spp1, an effect reversed by a neutralizing Spp1 antibody, which also significantly reduced basal KPC cell migration (FIG. 4D). Spp1 promotes migration through binding to integrins and CD44 receptors, which are also upregulated in KPC cells (Table 3), and we found that anti-CD44 antibody decreased Spp1-mediated migration compared to IgG-control-treated counterparts (FIG. 4D). Finally, if Spp1 secretion is important for distant colonization, then circulating plasma levels should be high in mice with metastases regardless of genotype. Circulating Spp1 was indeed elevated in the majority of KPC mice, and the relatively few KPDC mice, that developed significant metastatic disease burdens (FIG. 4E).

Some genes in the ECM profile, including the top-ranked one, Col6a1, were less familiar (Table 3). COL6A1 binds fibrillar type I collagen (Bonaldo et al., Biochem. 29:1245, 1990), itself complicit in PDA aggressiveness and dissemination (e.g., Armstrong et al., Clin. Cancer Res. 10:7427, 2004). COL6A1 is elevated in the circulating proteome of PDA patients (Yu et al., J. Proteome Res. 8:1565, 2009), and we found two RUNX3 consensus-binding sites in the COL6A1/Col6a1 promoter region (not shown). Little is known, however, about the specific function(s) of this gene in cancer and, in particular, in PDA. We first confirmed that Col6a1 is differentially expressed in KPC and KPDC cells (FIGS. 4F and 11D). Overexpression of Runx3 in KPDC cells increased Col6a1 transcript and protein levels (FIGS. 4G and 11D), whereas Runx3 silencing in KPC cells reduced them (FIGS. 4H and 11E). We next tested whether Col6a1 could act as an autocrine enabler of dissemination. KPC cells secrete higher levels of Col6a1 than KPDC cells (FIG. 41), and overexpressing Col6a1 in KPDC cells increased migration and silencing Col6a1 in KPC cells inhibited it (FIGS. 4J and 11F-I). Thus, in addition to providing a potentially critical component of the three-dimensional architecture of a developing tumor, Col6a1 also appears to directly stimulate cell motility. Finally, overexpression of Col6a1 in KPDC or knockdown in KPC cells reversed their respective abilities to seed lung metastases after intravenous injection (FIGS. 4K and 4L). Overall, these results show that Runx3 serves to both expel the seed and prepare the soil by directly stimulating cell migration and dissemination, and by inducing the expression of a repertoire of proteins that can reengineer the extracellular matrix to enhance distant colonization.

Heterozygous Deletion of Dpc4 Increases Proliferation in the Context of Oncogenic Kras^{G12D} and Trp53^{R172H}

The increased primary tumor size at the expense of metastatic disease seen in KPDC compared with KPC PDA could be explained by increased proliferation, decreased apoptosis, or both. Ki-67 expression was significantly increased in KPDC versus KPC autochthonous tumors and apoptosis was similarly negligible in both contexts (FIGS. 5A-5E). In culture, KPDC PDA cells were more proliferative than KPC cells (FIG. 5F) and had similarly low apoptotic rates (not shown). KPDC cells were also somewhat less sensitive to growth-arrest by TGFβ (FIG. 5F). Substantiating its sometime designation as a TSG, silencing Runx3 in KPC cells unleashed proliferation and overexpression of Runx3 in KPDC cells inhibited it (FIGS. 5G and 5H). We identified multiple consensus Runx3 binding sites in the p21^{Cip1/WAF1} promoter region and confirmed that Runx3 stimulated its expression (FIG. 51), as previously demonstrated in gastric epithelial cells Chi et al., Mol. Cell. Biol. 25:8097, 2005).

### Rare Metastasis of KPDC PDA Requires LOH of Dpc4

The above findings prompted an analysis of DPC4 status in paired primary tumors and metastases in the few KPDC animals that did develop disseminated disease. The primary tumors revealed focal areas of DPC4 loss, suggesting a potential source for the disseminated cells (FIGS. 6A and 6B). In addition, a strong inverse correlation was observed between Dpc4 and Runx3 expression in primary tumors and metastases (FIGS. 6C and 6D): complete loss of DPC4 was associated with elevated levels of RUNX3, whereas retained DPC4 corresponded with low-to-undetectable Runx3 (FIG. 12A). These observations suggested that completing the loss of DPC4 in PDA cells that had undergone a first (heterozygous) “hit” may confer metastatic potential on cells that previously lacked it, perhaps by increasing Runx3.

To directly investigate the effects of homozygous loss of DPC4 on autochthonous PDA, we generated and characterized Kras^{LSL-G12D/+}; Trp53^{LSL-R172H/+}; Dpc4^{flox/flox}; p48^{Cre/+} (KPDDC) quintuple mutant mice. KPDDC mice had a median survival of 125 days, significantly shorter than both KPDC and KPC cohorts (not shown). Large, multinodular pancreas tumors were found to develop from PanIN precursors (FIGS. 12B and 12C) and were histologically indistinguishable from KPDC or KPC PDA (FIGS. 12D and 12E). KPDDC primary tumors grew much faster than KPDC and KPC PDA, as may be expected from complete loss of canonical TGFβ signaling (FIG. 12F). In addition, KPDDC mice exhibited metastases to the liver and lungs at similar frequencies (73%) to KPC mice, a capability reflected in the elevated levels of Runx3 in KPDDC primary tumors (FIG. 12G) and purified cells (FIG. 12H). This combination of unfettered proliferation and potent dissemination explains the rapid demise of KPDDC mice and is consistent with the subset of especially aggressive human PDA with a similar mutational profile (Yachida et al., Clin. Cancer Res. 18:6339, 2012). In keeping with the role of Runx3 in modulating ECM gene expression to promote metastasis, Spp1 and Col6a1 were also elevated in KPDDC compared to KPDC cells. Despite their considerable metastatic competency in vivo, KPDDC cells were nevertheless notably resistant to induction of EMT by TGFβ in vitro (FIGS. 6E and 12H). Collectively, these findings evidence a biphasic dependency of metastatic potential on DPC4—mirrored in Runx3 levels—with heterozygous mutation retarding, and subsequent LOH rescuing, the ability to disseminate (FIG. 6F).

### Multiple Inputs Regulate Runx3 Levels

To test the effects KPC, KPDC, and KPDDC mutations on RUNX3 expression, cells were incubated in cycloheximide to arrest protein synthesis and followed the kinetics of RUNX3 degradation: protein levels were indeed most stable in KPC cells, and the already low Runx3 levels in KPDC cells dropped even further (FIGS. 121 and 12J). These findings show that homozygous levels of DPC4 promoted Runx3 synthesis and that heterozygous levels were sufficient to promote Runx3 protein degradation. We predicted therefore that restoring Dpc4 expression in KPDC cells would increase RUNX3 transcript levels and that partial knockdown in KPC would decrease them, both of which were observed (FIGS. 12K and 12L). That the basal protein levels are both elevated and more stable in KPDDC than KPDC cells is also consistent with this interpretation, but suggests the presence of at least one more input into Runx3 transcription, perhaps Runx3 itself. In this scenario, once the concentration of the Runx3 transcription factor achieves a critical threshold, it further promotes its own transcription. The RUNX3 promoter contains three consensus Runx3 binding sites, prompting us to test this hypothesis directly in reporter assays with increasing amounts of exogenous Runx3 (FIG. 12M). As shown, once Runx3 rises above a certain level, it rapidly increases its own transcription.

### RUNX3 Drives Metastasis and Attenuates Proliferation in Human PDA

To determine whether RUNX3 served similar roles in human pancreas cancer, we first assessed protein levels in a panel of well-characterized human PDA cell lines (FIG. 7A). There are wide differences across these extensively passaged cell lines in the mutational status of the principal TSG, and even among multiple reports of a single line (Deer et al., Pancreas 39:425, 2010). Moreover, as disease behavior may also reflect the chronologic sequence in which mutations arise (see Izeradjene et al., 2007) and we cannot know that history, we asked whether RUNX3 levels alone could be used to infer metastatic capability. We chose two of the highest (CFPAC-1 and Panc-1) and one of the lowest (MiaPaCa-2) RUNX3-expressing lines for further study (FIG. 7A). We note that although MiaPaCa-2 cells are WT for DPC4, they contain markedly decreased—but not completely absent—levels of TGFβR2 (not shown), suggesting an alternative way to attenuate DPC4-mediated signaling and potentially produce the same functional outcome.

Basal levels of RUNX3 predicted migration, as MiaPaCa-2 cells migrated less well than either CFPAC-1 or Panc-1 cells (FIGS. 7B and 7C). The respective responses of these cell lines to TGFβ also followed the pattern seen with murine PDA cells: TFGβ modestly inhibited MiaPaCa-2 cell migration, as it did with KPDC cells, whereas it promoted or left unaffected the high basal migration of CFPAC-1 and Panc-1 cells, similar to KPC cells. Overexpression of RUNX3 in MiaPaCa-2 cells (FIG. 13A) greatly increased migration (FIGS. 7B and 13B). Conversely, depletion of RUNX3 in both Panc-1 cells and CFPAC-1 cells inhibited migration (FIGS. 7C and 13C and 13D). Anchorage-independent growth of MiaPaCa-2 and CFPAC-1 cells was similarly influenced by RUNX3 overexpression and knockdown, respectively (FIGS. 13E and 13F). MiaPaCa-2 cells overexpressing RUNX3 developed a significantly greater metastatic burden after intravenous inoculation into NOD/SCID mice than vector-control cells (FIGS. 7D and 7E). Conversely, targeted depletion of RUNX3 in Panc-1 cells diminished their overall metastatic potential. Although both control and RUNX3-knockdown Panc-1 cells produced a comparable metastatic burden in the lungs (FIG. 13G), the ability to seed and support secondary liver metastases was significantly impaired by RUNX3 depletion as reflected in the distinct gross pathology (FIG. 7F) and liver weights between the two groups: 4.3+/−0.6 g (n=4) vs. 0.53+/−0.06 g (n=3; p<0.005), respectively. SPP1 and COL6A1 expression levels were also influenced as expected by modulating RUNX3 expression in these respective human cell lines, and COL6A1 overexpression stimulated migration (FIGS. 13H-13J). Thus, the RUNX3-dependent metastatic drive and responses to TGFβ observed in human PDA cells paralleled the behaviors of their murine counterparts.

The effect of variable RUNX3 expression on proliferation in human tissue samples was then examined. Higher levels of proliferation were observed in regions of human PDA that lacked RUNX3 expression as compared to regions expressing RUNX3 (FIG. 15A). The effect of RUNX3-knockdown in cultured CFPAC-1 cells (which show high levels of RUNX3 expression) and of RUNX3 overexpression in cultured MiaPaCa2 cells (which show low levels of RUNX3 expression) was then tested. shRNA-mediated knockdown of RUNX3 expression led to a pronounced increase in proliferation, while overexpressing RUNX3 in an otherwise RUNX3-deficient context led to similar a similar decrease in proliferation (FIGS. 15B and 15C, respectively). These data indicate that DPC4-mediated control of RUNX3 expression governs the switch between proliferation and metastasis in human PDA. Thus, RUNX3 levels and DPC4 may be predictive of disease progression and thus useful to inform therapeutic approaches.

### RUNX3 in Survival and Disease Behavior in Human PDA

RUNX3 levels in primary tumors were investigated to determine whether this influenced disease behavior and survival after surgery and even predict response to various forms of therapy. Given the predilection for distant relapse after surgery, primary PDA epithelial cells were examined for RUNX3 levels and prognosis. In a cohort of PDA patients who underwent definitive resection (n=88), RUNX3 levels correlated with survival (FIGS. 7G, 13L and 13M). Since this data set did not include information on site of relapse, a separate annotated cohort assembled by The International Cancer Genome Consortium (ICGC), which included whole-tissue transcriptome analyses and data on relapse pattern and survival after surgery, was interrogated. In this case, RUNX3 transcript levels did not correlate with either survival or relapse site. Consequently, the downstream targets of epithelial RUNX3 expression was assessed, and elevated tissue levels of SPP1 and COL6A1 was observed in patients who developed distant as opposed to local disease recurrence after surgery (FIG. 7H).

Different treatment modalities in the post-operative and pre-operative settings were investigated to determine whether patients with lower levels of RUNX3 might respond better to radiotherapy, given the higher risk of local relapse, whereas patients with high RUNX3 might fare better with systemic therapy. The use of local radiation favored patients with low RUNX3 (FIG. 7I). Conversely, patients with high RUNX3 benefited most from systemic treatment, while those with low RUNX3 did not (and may even have been harmed given that low RUNX3 should otherwise portend a better prognosis).

The various embodiments described above can be combined to provide further embodiments. All of the U.S. patents, U.S. patent application publications, U.S. patent applications, foreign patents, foreign patent applications and non-patent publications referred to in this specification and/or listed in the Application Data Sheet, including but not limited to U.S. Patent Application No. 62/163,842, are incorporated herein by reference, in their entirety. Aspects of the embodiments can be modified, if necessary to employ concepts of the various patents, applications and publications to provide yet further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

What is claimed is:

1. A method for diagnosing or detecting metastatic potential of pancreas cancer cells, comprising determining whether pancreas cancer cells from a mammalian subject comprise:
a homozygous positive genotype for DPC4 or have a single copy of DPC4, wherein the single copy is positive for DPC4, and have a RUNX3 expression level that is increased compared to normal cells from the subject, the pancreas cancer cells thereby having an increased metastatic potential as compared to tumor growth potential; or
(ii) a heterozygous genotype for DPC4 and have a RUNX3 expression level that is similar to normal cells from the subject, the pancreas cancer cells thereby not having an increased metastatic potential as compared to tumor growth potential; or
(iii) homozygous null genotype for DPC4 or have a single copy of DPC4, wherein the single copy is null for DPC4, and have a RUNX3 expression level that is increased as compared to normal cells from the subject, the pancreas cancer cells thereby having metastatic potential and/or tumor growth potential.

2. A method for diagnosing or detecting metastatic potential of pancreas cancer cells, comprising determining whether the pancreas cancer cells from a mammalian subject have:
a RUNX3 expression level that is increased as compared to normal cells from the subject, and an expression level of Spp1, Col6a1, or both, that is increased as compared to normal cells from the subject, the pancreas cancer cells thereby having an increased metastatic potential as compared to tumor growth potential; or
(ii) a RUNX3 expression level that is increased as compared to normal cells from the subject, and an expression level of Spp1, Col6a1, or both, that is decreased or similar as compared to normal cells from the subject, the pancreas cancer cells thereby not having an increased metastatic potential as compared to tumor growth potential.

3. A method for reducing the risk of metastatic spread of pancreas cancer cells, comprising:
treating a subject with a neoadjuvant therapy, a tumor resection procedure, and optionally an adjuvant therapy when the subject has pancreas cancer cells that comprise a homozygous positive genotype for DPC4 or a single copy of DPC4, wherein the single copy is positive for DPC4, and that have a RUNX3 expression level that is increased as compared to normal cells, thereby reducing the risk of metastatic spread; or
(ii) treating a subject with a tumor resection procedure, an adjuvant therapy, and optionally a neoadjuvant therapy when the subject has pancreas cancer cells that comprise a homozygous null genotype for DPC4 or have a single copy of DPC4, wherein the single copy is null for DPC4, and that have a RUNX3 expression level that is increased as compared to normal cells, thereby reducing the risk of metastatic spread.

4. The method of claim 3, wherein the subject of subpart (i) is treated with a neoadjuvant therapy comprising a systemic therapy and an adjuvant therapy comprising a systemic therapy.

5. A method for reducing the risk of local recurrence and/or proliferation of pancreas cancer, comprising treating a subject with a tumor resection procedure and optionally an adjuvant therapy when pancreas cancer cells from the subject comprise:
a heterozygous genotype for DPC4 and have a RUNX3 expression level that is similar as compared to normal cells from the subject; or
(ii) a homozygous null genotype for DPC4 or have a single copy of DPC4, wherein the single copy is null for DPC4, and have a RUNX3 expression level that is increased as compared to normal cells from the subject,
thereby reducing the risk of local recurrence and/or proliferation.

6. The method of any one of claims 1-5, wherein the expression level is determined using one or more of: RT-PCR, in situ hybridization, fluorescence-labeled oligonucleotide probes, radioactively labeled oligonucleotide probes, Northern blot, immunostaining, immunoprecipitation, fluorescence-labeling, BCA, or Western blot.

7. The method of any one of claim 1 or 3-6, wherein the genotype for DPC4 is determined by PCR followed by one or more of gel electrophoresis or DNA sequencing.

8. The method of any one of claims 3-5, wherein one or both of the neoadjuvant therapy and the adjuvant therapy comprise a chemotherapy, combined chemotherapies, radiotherapy, biologic therapy, hormonal therapy, or any combination thereof.

9. The method of any one of claims 3-5, wherein one or both of the neoadjuvant therapy and the adjuvant therapy further comprises administering to the subject an expression or activity inhibitor of RUNX3, Bmpr1a, Smad5, Tgfb3, Smad4, Bmp1, Itgb7, Tgfb1, Bmper, Ltbp1, Ltbp2, Id1, Tgfbi, Dlx2, cyclin D, cyclin E, Ctgf, Selp, Timp2, Col5a1, Ncam1, Thbs3, Mmp11, Sgce, Fn1, Vcan, Ecm1, Adamts1, Mmp2, Thbs1, Fbln1, Cdhh2, Mmp10, Timp3, Spp1, Col6a1, Sparc, Vcam1, or any combination thereof.

10. The method of claim 8, wherein the chemotherapy comprises an alkylating agent, an antimetabolite, a taxane, an anthracycline, bleomycin, mytomycin, actinomycin, hydroxyurea, a topoisomerase inhibitor, an antibody, a vinca alkaloid, cyclophosphamide, prednisone, leucovorin, oxaliplatin, or a hyaluronidase.

11. The method of claim 8, wherein the chemotherapy comprises 5-fluorouracil, capecitabine, gemcitabine, bendamustine, cisplatin, irinotecan, paclitaxel, docetaxel, leucovirin, nanoparticle albumin bound (nab)-paclitaxel, docetaxel, capecitabine, oxaplatin, or any combination thereof.

12. The method of claim 8, wherein the biologic therapy comprises an antibody, an scFv, a nanobody, a fusion protein, a tyrosine kinase inhibitor, an immunoreactive T cell, an immunoreactive Natural Killer cell, or any combination thereof.

13. The method of claim 12, wherein the antibody comprises cetuximab, trastuzumab, bevacizumab, alemtuzumab, gemtuzumab, panitumumab, rituximab, tositumomab, anti-CD44 antibody, anti-Spp1 antibody, or any combination thereof.

14. The method of any one of claims 3-13, wherein the tumor resection procedure comprises a standard Whipple procedure or a pylorus preserving Whipple procedure.

15. The method of any one of claims 1-14, wherein the subject has early-stage pancreas cancer.

16. A kit for use in for diagnosing or detecting, in pancreas cancer cells from a mammalian subject, a metastatic potential, a tumor growth potential, or both, comprising:
an oligonucleotide primer set specific for DPC4;
a binding agent specific for RUNX3;
optional instructions for using the primer and the detectable agent;
optional reagents for performing a PCR reaction; and
optional reagents for performing a binding reaction using the detectable agent,
wherein an increased metastatic potential as compared to tumor growth potential is present when pancreas cancer cells from a mammalian subject comprise a homozygous positive genotype for DPC4 or have a single copy of DPC4, wherein the single copy is positive for DPC4, and have a RUNX3 expression level that is increased as compared to normal cells from the subject; or
wherein an increased tumor growth potential as compared to metastatic potential is present when pancreas cancer cells from the mammalian subject comprise a heterozygous genotype for DPC4 and have a RUNX3 expression level that is similar to normal cells from the subject; or
wherein a metastatic potential and/or a tumor growth potential is present when pancreas cancer cells from the mammalian subject comprise a homozygous null genotype for DPC4 or have a single copy of DPC4, wherein the single copy is null for DPC4, and have a RUNX3 expression level that is increased as compared to normal cells from the subject.

17. The kit of claim 16, wherein the binding agent specific for RUNX3 comprises a nanobody or a binding fragment thereof, an antibody or a binding fragment thereof, or a T cell receptor or a binding fragment thereof.

18. The kit of claim 16 or 17, wherein the binding agent is conjugated to a detectable agent.

19. The kit of claim 18, wherein the detectable agent is detectable by one or more of: a colorimetric assay, fluorescence imaging, an enzymatic assay, spectrophotometry, mass spectroscopy, or radiation imaging.

20. A method for treating pancreas cancer in a mammalian subject, the method comprising:
(a) requesting a test to determine (i) a genotype for DPC4 and (ii) an expression level of RUNX3, in pancreas cancer cells from the subject; and
(b) administering to the subject a main therapy in combination with a neoadjuvant therapy, an adjuvant therapy or both, wherein the main therapy comprises tumor resection, and:
(i) the neoadjuvant therapy comprises a systemic therapy when the pancreas cancer cells comprise a homozygous positive genotype for DPC4 or have a single copy of DPC4, wherein the single copy is positive for DPC4, and have a RUNX3 expression level that is increased as compared to normal cells from the subject, and optionally administering an adjuvant therapy comprising a systemic therapy; or
(ii) the optional neoadjuvant therapy comprises a localized therapy when the pancreas cancer cells comprise a heterozygous genotype for DPC4 and have a RUNX3 expression level that is similar or decreased as compared to normal cells from the subject, and, and optionally administering an adjuvant therapy comprising a systemic therapy; or
(iii) the adjuvant therapy comprises a systemic therapy when the pancreas cancer cells comprise a homozygous null genotype for DPC4 or have a single copy of DPC4, wherein the single copy is null for DPC4, and have a RUNX3 expression level that is increased as compared to normal cells from the subject, and optionally administering to the subject, prior to the main therapy, a neoadjuvant therapy comprising a localized therapy.

21. The method of claim 20, comprising step (a) and step (b)(ii).

22. The method of claim 20 or 21, wherein the expression level is determined using one or more of RT-PCR, in situ hybridization, fluorescence-labeled oligonucleotide probes, radioactively labeled oligonucleotide probes, Northern blot, immunostaining, immunoprecipitation, fluorescence-labeling, BCA, or Western blot, and the genotype for DPC4 is determined using PCR followed by one or more of gel electrophoresis or DNA sequencing.

23. A method for treating pancreas cancer in a mammalian subject, the method comprising:
(a) requesting a test to determine a RUNX3 expression level and a Col6a1 expression level in pancreas cancer cells from the subject; and
(b) administering to the subject a main therapy in combination with a neoadjuvant therapy, an adjuvant therapy or both, wherein the main therapy comprises tumor resection and:
(i) the neoadjuvant therapy comprises a systemic therapy and an optional adjuvant therapy comprises a systemic therapy when the pancreas cancer cells have expression levels of both RUNX3 and Col6a1 that are increased as compared to normal cells from the subject; or
(ii) an optional neoadjuvant therapy comprises a localized therapy and an adjuvant therapy comprises a systemic therapy when the pancreas cancer cells have a RUNX3 expression level that is increased as compared to normal cells from the subject and a Col6a1 expression level that is decreased or similar as compared to normal cells from the subject.

24. The method of claim 23, wherein the expression levels are determined using one or more of: RT-PCR, immunostaining, immunoprecipitation, fluorescence-labeling, BCA, or Western blot.

25. A method for treating pancreas cancer based on a DPC4 genotype and RUNX3 expression level, comprising administering to a subject a main therapy in combination with a neoadjuvant therapy, an adjuvant therapy, or both, wherein the main therapy comprises tumor resection, and:
(a) the neoadjuvant therapy comprises a systemic therapy and an optional adjuvant therapy comprises a systemic therapy when the pancreas cancer cells comprise a homozygous positive genotype for DPC4 or have a single copy of DPC4, wherein the single copy is positive for DPC4, and have a RUNX3 expression level that is increased as compared to normal cells from the subject; or
(b) an optional neoadjuvant therapy comprises a localized therapy and the adjuvant therapy comprises a systemic therapy when the pancreas cancer cells are heterozygous for DPC4, and have a RUNX3 expression level that is similar to normal cells from the subject; or
(c) an optional neoadjuvant therapy comprises a systemic therapy, and the adjuvant therapy comprises a systemic therapy, when the pancreas cancer cells from the subject comprise a homozygous null genotype for DPC4 or have a single copy of DPC4, wherein the single copy is null for DPC4, and have a RUNX3 expression level that is increased as compared to normal cells from the subject.

26. The method of any one of claims 20-25, wherein the neoadjuvant therapy, the adjuvant therapy, or both further comprise administering to the subject an expression or activity inhibitor of RUNX3, Bmpr1a, Smad5, Tgfb3, Smad4, Bmp1, Itgb7, Tgfb1, Bmper, Ltbp1, Ltbp2, Id1, Tgfbi, Dlx2, cyclin D, cyclin E, Ctgf, Selp, Timp2, Col5a1, Ncam1, Thbs3, Mmp11, Sgce, Fn1, Vcan, Ecm1, Adamts1, Mmp2, Thbs1, Fbln1, Cdhh2, Mmp10, Timp3, Spp1, Col6a1, Sparc, Vcam1, or any combination thereof.

27. The method of any one of claims claim 20-26, wherein the systemic therapy comprises chemotherapy, combined chemotherapies, biologic therapy, hormonal therapy, or any combination thereof.

28. The method of any one of claims 20-27, wherein the localized therapy comprises radiotherapy, chemotherapy, combined chemotherapies, hormonal therapy biologic therapy, or any combination thereof.

29. The method of claim 27 or 28, wherein the chemotherapy comprises an alkylating agent, an antimetabolite, a taxane, an anthracycline, bleomycin, mytomycin, actinomycin, hydroxyurea, a topoisomerase inhibitor, an antibody, a vinca alkaloid, cyclophosphamide, prednisone, leucovorin, oxaliplatin, or a hyaluronidnase, or any combination thereof.

30. The method of claim 27 or 28, wherein the chemotherapy comprises 5-fluorouracil, capecitabine, gemcitabine, bendamustine, cisplatin, irinotecan, paclitaxel, docetaxel, leucovirin, nanoparticle albumin bound (nab)-paclitaxel, docetaxel, capecitabine, oxaplatin, or any combination thereof.

31. The method of claim 27 or 28, wherein the biologic therapy comprises an antibody, an scFv, a nanobody, a fusion protein, a tyrosine kinase inhibitor, an immunoreactive T cell, an immunoreactive Natural Killer cell, or any combination thereof.

32. The method of claim 30, wherein the antibody comprises cetuximab, trastuzumab, bevacizumab, alemtuzumab, gemtuzumab, panitumumab, rituximab, tositumomab, anti-CD44 antibody, anti-Spp1 antibody, or any combination thereof.

33. The method of any one of claims 20-32, wherein the tumor resection comprises a standard Whipple procedure or a pylorus preserving Whipple procedure.

34. The method of any one of claims 20-33, wherein the subject has early-stage pancreas cancer.

35. The method of any one of claims 1-34, wherein the subject has PDA or a PDA precursor lesion.

36. The method of any one of claims 1-35, wherein the mammalian subject is a human.


SEQUENCE LISTING (17 sequences)

SEQ ID NO: 1 - DNA, 20 bp, Artificial Sequence
  Forward primer sequence
agagtttcacgctcacaatc

SEQ ID NO: 2 - DNA, 18 bp, Artificial Sequence
  Reverse primer sequence
ggagaaggggttcaggtc

SEQ ID NO: 3 - DNA, 19 bp, Artificial Sequence
  Forward primer sequence
ctgctgctacaagcctgct

SEQ ID NO: 4 - DNA, 21 bp, Artificial Sequence
  Reverse primer sequence
ccccataaggtttcagcctca

SEQ ID NO: 5 - DNA, 22 bp, Artificial Sequence
  Forward primer sequence
gagctgtttgcagacaaagttc

SEQ ID NO: 6 - DNA, 20 bp, Artificial Sequence
  Reverse primer sequence
ccctggcacatgaatcctgg

SEQ ID NO: 7 - DNA, 22 bp, Artificial Sequence
  Forward primer sequence
tttcactccaatcgtccctaca

SEQ ID NO: 8 - DNA, 23 bp, Artificial Sequence
  Reverse primer sequence
tcagtccataagccaagctatca

SEQ ID NO: 9 - DNA, 20 bp, Artificial Sequence
  Forward primer sequence
acaccgactgcgctatcaag

SEQ ID NO: 10 - DNA, 22 bp, Artificial Sequence
  Reverse primer sequence
cggtcaccacaatcaggtactt

SEQ ID NO: 11 - DNA, 21 bp, Artificial Sequence
  Forward primer sequence
cccaccgtgttcttcgacatt

SEQ ID NO: 12 - DNA, 22 bp, Artificial Sequence
  Reverse primer sequence
ggacccgtatgctttaggatga

SEQ ID NO: 13 - DNA, 21 bp, Artificial Sequence
  Forward primer sequence
ctccattgactcgaacgactc

SEQ ID NO: 14 - DNA, 23 bp, Artificial Sequence
  Reverse primer sequence
caggtctgcgaaacttcttagat

SEQ ID NO: 15 - DNA, 21 bp, Artificial Sequence
  shRNA sequences (sense strands)
tagagaggacattgatttggg

SEQ ID NO: 16 - DNA, 21 bp, Artificial Sequence
  shRNA sequence (sense strand)
tacacaaagcaaagagtgtgc

SEQ ID NO: 17 - DNA, 19 bp, Artificial Sequence
  shRNA sequence (sense strand)
cggcagaagctggaggacc